SEQUENCE LISTING (2 sequences)

SEQ ID NO: 1 - protein, 132 aa, synthetic construct
  REGION 1..132: note = IL-2 conjugate
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 2 - protein, 132 aa, synthetic construct
  REGION 1..132: note = IL-2 conjugate
  SITE 64: note = N6-((2-azidoethoxy)-carbonyl)-L-lysine stably-conjugated to PEG via DBCO-mediated click chemistry
PTSSSTKKTQLQLEHLLLDLQMILNGINNYKNPKLTRMLTFKFYMPKKATELKHLQCLEEELKKLEEVLNLAQSKNFHLRPRDLISNINVIVLELKGSETTFMCEYADETATIVEFLNRWITFSQSIISTLT

(19) United States
(12) Patent Application Publication
(10) Pub. No.: US 2023/0277627 A1
(43) Pub. Date: Sep. 7, 2023

(54) IMMUNO ONCOLOGY COMBINATION THERAPY WITH IL-2 CONJUGATES AND PEMBROLIZUMAB

(71) Applicant: Synthorx, Inc., La Jolla, CA (US)

(72) Inventors: Carolina E. CAFFARO, San Diego, CA (US); Joseph LEVEQUE, San Diego, CA (US); Marcos MILLA, Carlsbad, CA (US); Jerod PTACIN, San Diego, CA (US); Laura SHAWVER, Edmonds, WA (US)

(73) Assignee: Synthorx, Inc., La Jolla, CA (US)

(21) Appl. No.: 18/296,710

(22) Filed: Apr. 6, 2023

Related U.S. Application Data
(63) Continuation of application No. PCT/US2021/054225, filed on Oct. 8, 2021.
(60) Provisional application No. 63/090,033, filed on Oct. 9, 2020.
(60) Provisional application No. 63/158,669, filed on Mar. 9, 2021.
(60) Provisional application No. 63/173,114, filed on Apr. 9, 2021.

(51) Int. Cl. A61K 38/20; A61P 35/00; A61K 47/68; C07K 16/28; A61K 47/60

(52) U.S. Cl. CPC A61K 38/2013; A61P 35/00; A61K 47/6813; C07K 16/2896; A61K 47/60

(57) ABSTRACT

Disclosed herein are methods for treating a cancer in a subject in need thereof, comprising administering IL-2 conjugates in combination with pembrolizumab.

### CROSS-REFERENCE TO RELATED APPLICATIONS

This application is a continuation of International Application No. PCT/US2021/054225, filed on Oct. 8, 2021, which claims priority to U.S. Provisional Application No. 63/090,033, filed on Oct. 9, 2020, U.S. Provisional Application No. 63/158,669, filed on Mar. 9, 2021, and U.S. Provisional Application No. 63/173,114, filed on Apr. 9, 2021, the disclosure of each of which is hereby incorporated by reference in its entirety.

### SEQUENCE LISTING

This application contains a Sequence Listing which has been submitted electronically in XML format and is hereby incorporated by reference in its entirety. Said XML copy, created on Mar. 14, 2023, is named “2023-03-14_01183-0097-00US_ST26” and is 4,269 bytes in size.

### BACKGROUND OF THE DISCLOSURE

Distinct populations of T cells modulate the immune system to maintain immune homeostasis and tolerance. For example, regulatory T (Treg) cells prevent inappropriate responses by the immune system by preventing pathological self-reactivity while cytotoxic T cells target and destroy infected cells and/or cancerous cells. In some instances, modulation of the different populations of T cells provides an option for treatment of a disease or indication.

Cytokines comprise a family of cell signaling proteins such as chemokines, interferons, interleukins, lymphokines, tumor necrosis factors, and other growth factors playing roles in innate and adaptive immune cell homeostasis. Cytokines are produced by immune cells such as macrophages, B lymphocytes, T lymphocytes and mast cells, endothelial cells, fibroblasts, and different stromal cells. In some instances, cytokines modulate the balance between humoral and cell-based immune responses.

Interleukins are signaling proteins that modulate the development and differentiation of T and B lymphocytes, cells of the monocytic lineage, neutrophils, basophils, eosinophils, megakaryocytes, and hematopoietic cells. Interleukins are produced by helper CD4+ T and B lymphocytes, monocytes, macrophages, endothelial cells, and other tissue residents.

In some instances, interleukin 2 (IL-2) signaling is used to modulate T cell responses and subsequently for treatment of a cancer. Accordingly, in one aspect, provided herein are methods of treating cancer in a subject comprising administering an IL-2 conjugate in combination with the anti-PD-1 antibody pembrolizumab.

### SUMMARY OF THE DISCLOSURE

Described herein are methods of treating cancer in a subject in need thereof, comprising administering to a subject (a) about 8 μg/kg, 16 μg/kg, 24 μg/kg, or 32 μg/kg of an IL-2 conjugate, and (b) pembrolizumab, wherein the IL-2 conjugate comprises the amino acid sequence of SEQ ID NO: 1 having an unnatural amino acid residue described herein at position 64, e.g., the amino acid sequence of SEQ ID NO: 2.

Exemplary embodiments include the following.

Embodiment 1. A method of treating a cancer in a subject in need thereof, comprising administering to the subject (a) about 8 μg/kg, 16 μg/kg, 24 μg/kg, or 32 μg/kg of an IL-2 conjugate, and (b) pembrolizumab, wherein the IL-2 conjugate comprises the amino acid sequence of SEQ ID NO: 1 wherein the amino acid at position P64 is replaced by the structure of Formula (IA):

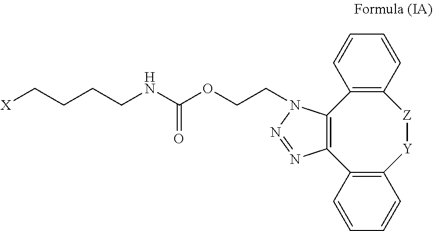

wherein:
Z is CH₂ and Y is

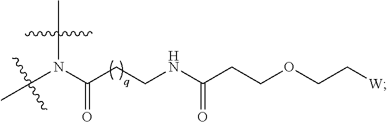

Y is CH₂ and Z is

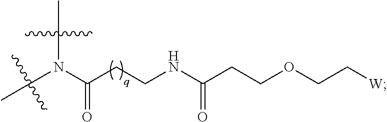

Z is CH₂ and Y is

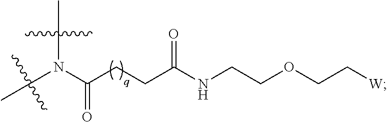

or
Y is CH₂ and Z is

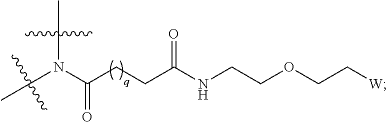

W is a PEG group having an average molecular weight of about 25 kDa-35 kDa;
q is 1, 2, or 3;
X is an L-amino acid having the structure:

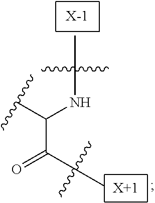 [SMILES: "X-1" ; *NC(*)C(*)=O ; "X+1"]

X−1 indicates the point of attachment to the preceding amino acid residue; and
X+1 indicates the point of attachment to the following amino acid residue.

Embodiment 2. An IL-2 conjugate for use in a method of treating a cancer in a subject in need thereof, the method comprising administering to the subject (a) about 8 μg/kg, 16 μg/kg, 24 μg/kg, or 32 μg/kg of the IL-2 conjugate, and (b) pembrolizumab, wherein the IL-2 conjugate comprises the amino acid sequence of SEQ ID NO: 1 wherein the amino acid at position P64 is replaced by the structure of Formula (IA):

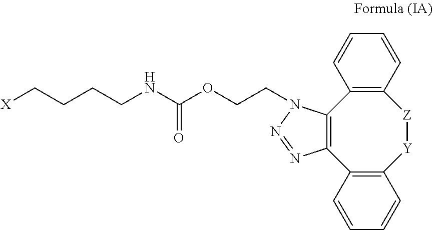

wherein:
Z is CH₂ and Y is

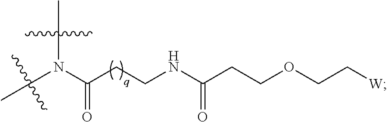

Y is CH₂ and Z is

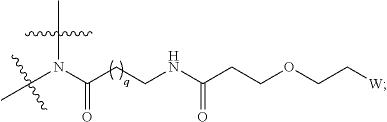

Z is CH₂ and Y is

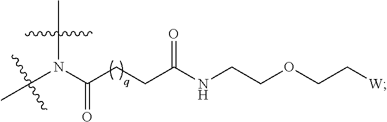

or
Y is CH₂ and Z is

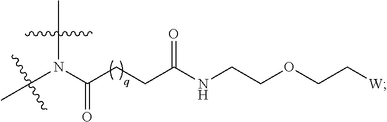

W is a PEG group having an average molecular weight of about 25 kDa-35 kDa;
q is 1, 2, or 3;
X is an L-amino acid having the structure:

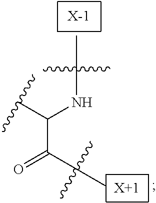 [SMILES: "X-1" ; *NC(*)C(*)=O ; "X+1"]

X−1 indicates the point of attachment to the preceding amino acid residue; and
X+1 indicates the point of attachment to the following amino acid residue.

Embodiment 3. Use of an IL-2 conjugate for the manufacture of a medicament for a method of treating a cancer in a subject in need thereof, the method comprising administering to the subject (a) about 8 μg/kg, 16 μg/kg, 24 μg/kg, or 32 μg/kg of the IL-2 conjugate, and (b) pembrolizumab, wherein the IL-2 conjugate comprises the amino acid sequence of SEQ ID NO: 1 wherein the amino acid at position P64 is replaced by the structure of Formula (IA):

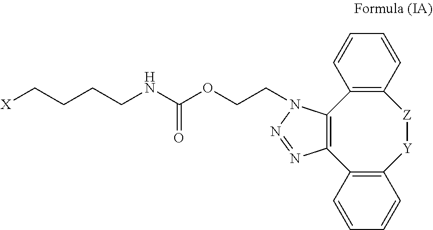

wherein:
Z is CH₂ and Y is

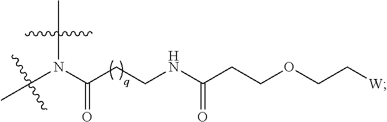

Y is CH₂ and Z is

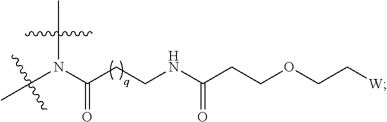

Z is CH₂ and Y is

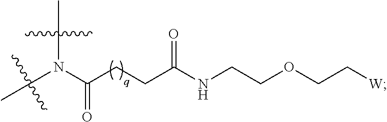

or
Y is CH₂ and Z is

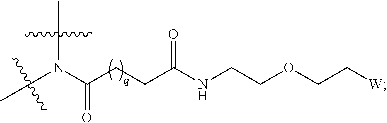

W is a PEG group having an average molecular weight of about 25 kDa-35 kDa;
q is 1, 2, or 3;
X is an L-amino acid having the structure:

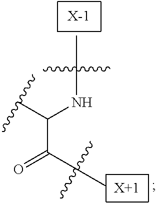 [SMILES: "X-1" ; *NC(*)C(*)=O ; "X+1"]

X−1 indicates the point of attachment to the preceding amino acid residue; and
X+1 indicates the point of attachment to the following amino acid residue.

Embodiment 4. The method, IL-2 conjugate for use, or use of any one of embodiments 1-3, comprising administering to the subject about 8 μg/kg of the IL-2 conjugate.

Embodiment 5. The method, IL-2 conjugate for use, or use of any one of embodiments 1-3, comprising administering to the subject about 16 μg/kg of the IL-2 conjugate.

Embodiment 6. The method, IL-2 conjugate for use, or use of any one of embodiments 1-3, comprising administering to the subject about 24 μg/kg of the IL-2 conjugate.

Embodiment 7. The method, IL-2 conjugate for use, or use of any one of embodiments 1-3, comprising administering to the subject about 32 μg/kg of the IL-2 conjugate.

Embodiment 8. The method, IL-2 conjugate for use, or use of any one of embodiments 1-7, wherein in the IL-2 conjugate Z is CH₂ and Y is

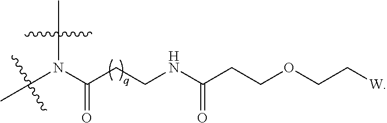

Embodiment 9. The method, IL-2 conjugate for use, or use of any one of embodiments 1-7, wherein in the IL-2 conjugate Y is CH₂ and Z is

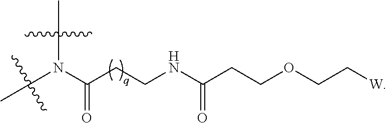

Embodiment 10. The method, IL-2 conjugate for use, or use of any one of embodiments 1-7, wherein in the IL-2 conjugate Z is CH₂ and Y is

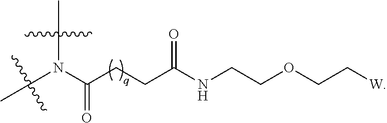

Embodiment 11. The method, IL-2 conjugate for use, or use of any one of embodiments 1-7, wherein in the IL-2 conjugate Y is CH₂ and Z is

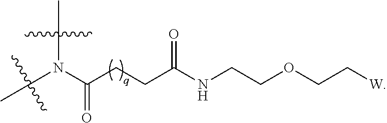

Embodiment 12. The method, IL-2 conjugate for use, or use of any one of embodiments 1-11, wherein in the IL-2 conjugate the PEG group has an average molecular weight of about 30 kDa.

Embodiment 13. The method, IL-2 conjugate for use, or use of any one of embodiments 1-7, wherein the structure of Formula (IA) has the structure of Formula (IVA) or Formula (VA), or is a mixture of Formula (IVA) and Formula (VA):

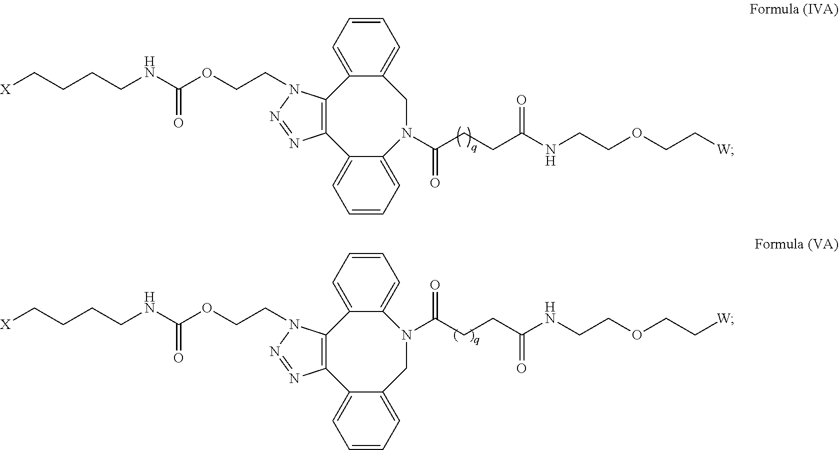

wherein:
W is a PEG group having an average molecular weight of about 25 kDa-35 kDa; and
q is 1, 2, or 3.

Embodiment 14. The method, IL-2 conjugate for use, or use of any one of embodiments 1-7, wherein the structure of Formula (IA) has the structure of Formula (XIIA) or Formula (XIIIA), or is a mixture of Formula (XIIA) and Formula (XIIIA)

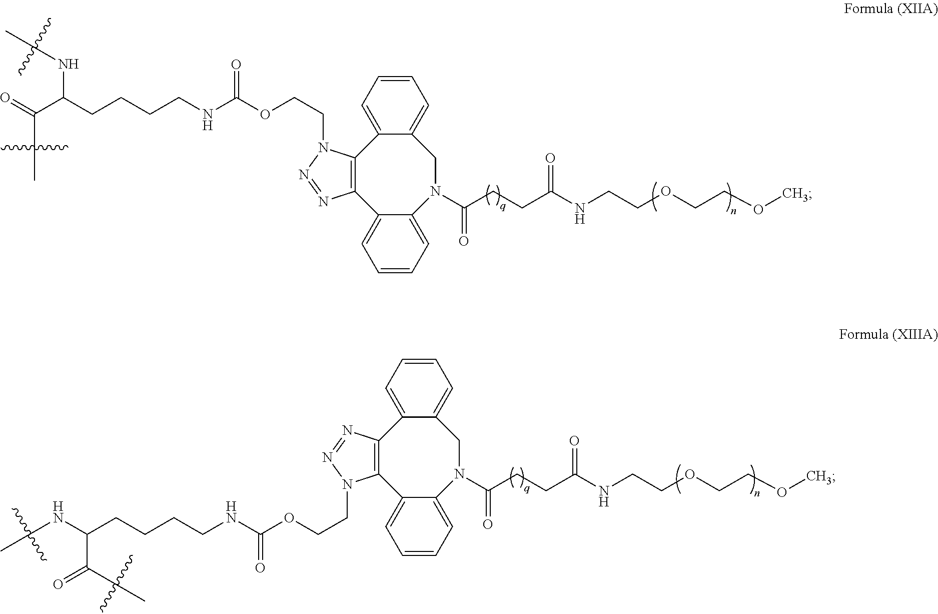

wherein:
n is an integer such that —(OCH₂CH₂)ₙ—OCH₃ has a molecular weight of about 30 kDa;
q is 1, 2, or 3; and
the wavy lines indicate covalent bonds to amino acid residues within SEQ ID NO: 1 that are not replaced.

Embodiment 15. The method, IL-2 conjugate for use, or use of any one of embodiments 1-14, wherein q is 1.

Embodiment 16. The method, IL-2 conjugate for use, or use of any one of embodiments 1-14, wherein q is 2.

Embodiment 17. The method, IL-2 conjugate for use, or use of any one of embodiments 1-14, wherein q is 3.

Embodiment 18. The method, IL-2 conjugate for use, or use of any one of embodiments 1-17, wherein the subject has a solid tumor cancer.

Embodiment 19. The method, IL-2 conjugate for use, or use of any one of embodiments 1-18, wherein the subject has a metastatic solid tumor.

Embodiment 20. The method, IL-2 conjugate for use, or use of any one of embodiments 1-19, wherein the subject has an advanced solid tumor.

Embodiment 21. The method, IL-2 conjugate for use, or use of any one of embodiments 1-17, wherein the subject has a liquid tumor.

Embodiment 22. The method, IL-2 conjugate for use, or use of any one of embodiments 1-21, wherein the subject has refractory cancer.

Embodiment 23. The method, IL-2 conjugate for use, or use of any one of embodiments 1-22, wherein the subject has relapsed cancer.

Embodiment 24. The method, IL-2 conjugate for use, or use of any one of embodiments 1-23, wherein the cancer is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, gastric cancer, colon cancer, colorectal cancer (CRC), cervical cancer, hepatocellular carcinoma (HCC), Merkel cell carcinoma (MCC), melanoma, small cell lung cancer (SCLC), esophageal, esophageal squamous cell carcinoma (ESCC), glioblastoma, mesothelioma, breast cancer, triple-negative breast cancer, prostate cancer, castrate-resistant prostate cancer, metastatic castrate-resistant prostate cancer, or metastatic castrate-resistant prostate cancer having DNA damage response (DDR) defects, bladder cancer, ovarian cancer, tumors of moderate to low mutational burden, cutaneous squamous cell carcinoma (CSCC), squamous cell skin cancer (SCSC), tumors of low- to non-expressing PD-L1, tumors disseminated systemically to the liver and CNS beyond their primary anatomic originating site, and diffuse large B-cell lymphoma (DLBCL).

Embodiment 25. The method, IL-2 conjugate for use, or use of any one of embodiments 1-24, wherein CD8+ cells are expanded at least 1.5-fold.

Embodiment 26. The method, IL-2 conjugate for use, or use of any one of embodiments 1-25, wherein NK cells are expanded at least about 5-fold.

Embodiment 27. The method, IL-2 conjugate for use, or use of any one of embodiments 1-26, wherein eosinophils are expanded no more than about 2-fold.

Embodiment 28. The method, IL-2 conjugate for use, or use of any one of embodiments 1-27, wherein CD4+ cells are expanded no more than about 2-fold.

Embodiment 29. The method, IL-2 conjugate for use, or use of any one of embodiments 1-28, wherein the expansion of CD8+ cells and/or NK cells is greater than the expansion of CD4+ cells and/or eosinophils.

Embodiment 30. The method, IL-2 conjugate for use, or use of any one of embodiments 1-29, wherein the IL-2 conjugate does not cause dose-limiting toxicity.

Embodiment 31. The method, IL-2 conjugate for use, or use of any one of embodiments 1-30, wherein the IL-2 conjugate does not cause severe cytokine release syndrome.

Embodiment 32. The method, IL-2 conjugate for use, or use of any one of embodiments 1-31, wherein the IL-2 conjugate does not cause vascular leak syndrome.

Embodiment 33. The method, IL-2 conjugate for use, or use of any one of embodiments 1-32, wherein the IL-2 conjugate is administered to the subject about once every two weeks, about once every three weeks, or about once every 4 weeks.

Embodiment 34. The method, IL-2 conjugate for use, or use of any one of embodiments 1-33, wherein the IL-2 conjugate and pembrolizumab are administered to the subject about once every two weeks, about once every three weeks, or about once every 4 weeks.

Embodiment 35. The method, IL-2 conjugate for use, or use of any one of embodiments 1-34, wherein the IL-2 conjugate is a pharmaceutically acceptable salt, solvate, or hydrate.

Embodiment 36. The method, IL-2 conjugate for use, or use of any one of embodiments 1-35, wherein pembrolizumab is administered at a dose of about 200 mg every 3 weeks.

Embodiment 37. The method, IL-2 conjugate for use, or use of any one of embodiments 1-36, wherein the IL-2 conjugate and pembrolizumab are administered separately.

Embodiment 38. The method, IL-2 conjugate for use, or use of embodiment 37, wherein the IL-2 conjugate and pembrolizumab are administered sequentially.

Embodiment 39. The method, IL-2 conjugate for use, or use of embodiment 37 or 38, wherein the IL-2 conjugate is administered before pembrolizumab.

Embodiment 40. The method, IL-2 conjugate for use, or use of embodiment 37 or 38, wherein the IL-2 conjugate is administered after pembrolizumab.

Embodiment 41. The method, IL-2 conjugate for use, or use of any one of embodiments 1-40, wherein the IL-2 conjugate is administered to the subject by subcutaneous administration.

Embodiment 42. The method, IL-2 conjugate for use, or use of any one of embodiments 1-40, wherein the IL-2 conjugate is administered to the subject by intravenous administration.

Embodiment 43. The method, IL-2 conjugate for use, or use of any one of embodiments 1-40 and 42, wherein the IL-2 conjugate and pembrolizumab are administered to the subject by intravenous administration.

Embodiment 44. The method, IL-2 conjugate for use, or use of any one of embodiments 1-43, wherein the subject has basal cell carcinoma.

Embodiment 45. The method, IL-2 conjugate for use, or use of any one of embodiments 1-43, wherein the subject has squamous cell carcinoma, optionally wherein the squamous cell carcinoma is head and neck squamous cell carcinoma.

Embodiment 46. The method, IL-2 conjugate for use, or use of any one of embodiments 1-43, wherein the subject has colorectal cancer.

Embodiment 47. The method, IL-2 conjugate for use, or use of any one of embodiments 1-43, wherein the subject has melanoma.

Embodiment 48. The method, IL-2 conjugate for use, or use of any one of embodiments 1-47, wherein the IL-2 conjugate has an in vivo half-life of about 10 hours.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows the change in peripheral CD8+ T_{eff} counts in the indicated subjects at specified times following administration of 8 μg/kg IL-2 conjugate and pembrolizumab. Here and elsewhere, designations such as “C1D1” indicate the treatment cycle and day (e.g., treatment cycle 1, day 1). “PRE” indicates the baseline measurement before administration; 24 HR indicates 24 hours after administration; and so on.

FIG. 1B shows the change in peak peripheral CD8+ T_{eff} cell expansion following administration of the first dose of IL-2 conjugate and pembrolizumab. Data is normalized to pre-treatment (C1D1) CD8+ T cell count. Listed values indicate median fold changes.

FIG. 1C shows the change in peripheral CD8+ T_{eff} counts in the indicated subjects at specified times following administration of 16 μg/kg IL-2 conjugate and pembrolizumab.

FIG. 2 shows the percentage of CD8+ T_{eff} cells expressing Ki67 in the indicated subjects at specified times following administration of 8 μg/kg IL-2 conjugate and pembrolizumab.

FIG. 3A shows the change in peripheral natural killer (NK) cell counts in the indicated subjects at specified times following administration of 8 μg/kg IL-2 conjugate and pembrolizumab.

FIG. 3B shows the change in peak peripheral NK cell expansion following administration of the first dose of IL-2 conjugate and pembrolizumab. Data is normalized to pre-treatment (C1D1) NK cell count. Listed values indicate median fold changes.

FIG. 3C shows the change in peripheral natural killer (NK) cell counts in the indicated subjects at specified times following administration of 16 μg/kg IL-2 conjugate and pembrolizumab.

FIG. 4 shows the percentage of NK cells expressing Ki67 in the indicated subjects at specified times following administration of 8 μg/kg IL-2 conjugate and pembrolizumab.

FIG. 5A shows the change in peripheral CD4+ T_{reg} counts in the indicated subjects at specified times following administration of 8 μg/kg IL-2 conjugate and pembrolizumab.

FIG. 5B shows the change in peak peripheral CD4+ T_{reg} cell expansion following administration of the first dose of IL-2 conjugate and pembrolizumab. Data is normalized to pre-treatment (C1D1) CD4+ T cell count. Listed values indicate median fold changes.

FIG. 5C shows the change in peripheral CD4+ T_{reg} counts in the indicated subjects at specified times following administration of 16 μg/kg IL-2 conjugate and pembrolizumab.

FIG. 6 shows the percentage of CD4+ T_{reg} cells expressing Ki67 in the indicated subjects at specified times following administration of 8 μg/kg IL-2 conjugate and pembrolizumab.

FIG. 7A shows the change in eosinophil cell counts in the indicated subjects at specified times following administration of 8 μg/kg IL-2 conjugate and pembrolizumab.

FIG. 7B shows the change in peak peripheral eosinophil cell expansion following administration of the first dose of IL-2 conjugate and pembrolizumab. Data is normalized to pre-treatment (C1D1) eosinophil cell count. Listed values indicate median fold changes.

FIG. 7C shows the change in eosinophil cell counts in the indicated subjects at specified times following administration of 16 μg/kg IL-2 conjugate and pembrolizumab.

FIG. 8A shows serum levels of IFN-γ, IL-5, and IL-6 in the indicated subjects at specified times following administration of 8 μg/kg IL-2 conjugate and pembrolizumab.

FIG. 8B shows the serum level of IL-5 following administration of 8 μg/kg IL-2 conjugate and pembrolizumab. BLQ=below limit of quantification. Data is plotted as mean (range BLQ to maximum value).

FIG. 8C shows the serum level of IL-6 following administration of 8 μg/kg IL-2 conjugate and pembrolizumab. BLQ=below limit of quantification. Data is plotted as mean (range BLQ to maximum value).

FIG. 8D shows serum levels of IFN-γ, IL-5, and IL-6 in the indicated subjects at specified times following administration of 16 μg/kg IL-2 conjugate and pembrolizumab.

FIG. 9A and FIG. 9B show mean concentrations of the IL-2 conjugate, administered at a dose of 8 μg/kg with pembrolizumab, after 1 and 2 cycles, respectively.

FIG. 9C and FIG. 9D show mean concentrations of the IL-2 conjugate, administered at a dose of 16 μg/kg with pembrolizumab, after 1 and 2 cycles, respectively.

FIG. 10 shows the peripheral CD8+ T_{eff} cell counts in the indicated subjects at specified times following administration of 24 μg/kg IL-2 conjugate and pembrolizumab.

FIG. 11 shows the peripheral NK cell counts in the indicated subjects at specified times following administration of 24 μg/kg IL-2 conjugate and pembrolizumab.

FIG. 12 shows the change in peripheral CD4+ T_{reg} cell counts in the indicated subjects at specified times following administration of 24 μg/kg IL-2 conjugate and pembrolizumab.

FIG. 13 shows the peripheral eosinophil cell counts in the indicated subjects at specified times following administration of 24 μg/kg IL-2 conjugate and pembrolizumab.

FIG. 14A and FIG. 14B show mean concentrations of the IL-2 conjugate, administered at a dose of 24 μg/kg with pembrolizumab, after 1 and 2 cycles, respectively.

FIG. 15 shows the levels of IFN-γ, IL-6, and IL-5 in the indicated subjects treated with 24 μg/kg of the IL-2 conjugate and pembrolizumab at specified times following administration of the IL-2 conjugate.

FIG. 16 shows the change in peripheral CD8+ T_{eff} cell counts in the indicated subjects at specified times following administration of 32 μg/kg IL-2 conjugate and pembrolizumab.

FIG. 17 shows the peripheral CD4+ T_{reg} cell counts in the indicated subjects at specified times following administration of 32 μg/kg IL-2 conjugate and pembrolizumab.

FIG. 18A and FIG. 18B show mean concentrations of the IL-2 conjugate, administered at a dose of 32 μg/kg with pembrolizumab, after 1 and 2 cycles, respectively.

FIG. 19 shows the levels of IFN-γ, IL-6, and IL-5 in the indicated subjects treated with 32 μg/kg of the IL-2 conjugate and pembrolizumab at specified times following administration of the IL-2 conjugate.

### DETAILED DESCRIPTION OF THE DISCLOSURE

### Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. To the extent any material incorporated herein by reference is inconsistent with the express content of this disclosure, the express content controls. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless the context requires otherwise. Furthermore, use of the term “including” as well as other forms, such as “include”, “includes,” and “included,” is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

As used herein, the terms “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “unnatural amino acid” refers to an amino acid other than one of the 20 naturally occurring amino acids. Exemplary unnatural amino acids are described in Young et al., “Beyond the canonical 20 amino acids: expanding the genetic lexicon,” J. of Biological Chemistry 285(15): 11039-11044 (2010), the disclosure of which is incorporated herein by reference.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

As used herein, “nucleotide” refers to a compound comprising a nucleoside moiety and a phosphate moiety. Exemplary natural nucleotides include, without limitation, adenosine triphosphate (ATP), uridine triphosphate (UTP), cytidine triphosphate (CTP), guanosine triphosphate (GTP), adenosine diphosphate (ADP), uridine diphosphate (UDP), cytidine diphosphate (CDP), guanosine diphosphate (GDP), adenosine monophosphate (AMP), uridine monophosphate (UMP), cytidine monophosphate (CMP), and guanosine monophosphate (GMP), deoxyadenosine triphosphate (dATP), deoxythymidine triphosphate (dTTP), deoxycytidine triphosphate (dCTP), deoxyguanosine triphosphate (dGTP), deoxyadenosine diphosphate (dADP), thymidine diphosphate (dTDP), deoxycytidine diphosphate (dCDP), deoxyguanosine diphosphate (dGDP), deoxyadenosine monophosphate (dAMP), deoxythymidine monophosphate (dTMP), deoxycytidine monophosphate (dCMP), and deoxyguanosine monophosphate (dGMP). Exemplary natural deoxyribonucleotides, which comprise a deoxyribose as the sugar moiety, include dATP, dTTP, dCTP, dGTP, dADP, dTDP, dCDP, dGDP, dAMP, dTMP, dCMP, and dGMP. Exemplary natural ribonucleotides, which comprise a ribose as the sugar moiety, include ATP, UTP, CTP, GTP, ADP, UDP, CDP, GDP, AMP, UMP, CMP, and GMP.

As used herein, “base” or “nucleobase” refers to at least the nucleobase portion of a nucleoside or nucleotide (nucleoside and nucleotide encompass the ribo or deoxyribo variants), which may in some cases contain further modifications to the sugar portion of the nucleoside or nucleotide. In some cases, “base” is also used to represent the entire nucleoside or nucleotide (for example, a “base” may be incorporated by a DNA polymerase into DNA, or by an RNA polymerase into RNA). However, the term “base” should not be interpreted as necessarily representing the entire nucleoside or nucleotide unless required by the context. In the chemical structures provided herein of a base or nucleobase, only the base of the nucleoside or nucleotide is shown, with the sugar moiety and, optionally, any phosphate residues omitted for clarity. As used in the chemical structures provided herein of a base or nucleobase, the wavy line represents connection to a nucleoside or nucleotide, in which the sugar portion of the nucleoside or nucleotide may be further modified. In some embodiments, the wavy line represents attachment of the base or nucleobase to the sugar portion, such as a pentose, of the nucleoside or nucleotide. In some embodiments, the pentose is a ribose or a deoxyribose.

In some embodiments, a nucleobase is generally the heterocyclic base portion of a nucleoside. Nucleobases may be naturally occurring, may be modified, may bear no similarity to natural bases, and/or may be synthesized, e.g., by organic synthesis. In certain embodiments, a nucleobase comprises any atom or group of atoms in a nucleoside or nucleotide, where the atom or group of atoms is capable of interacting with a base of another nucleic acid with or without the use of hydrogen bonds. In certain embodiments, an unnatural nucleobase is not derived from a natural nucleobase. It should be noted that unnatural nucleobases do not necessarily possess basic properties, however, they are referred to as nucleobases for simplicity. In some embodiments, when referring to a nucleobase, a “(d)” indicates that the nucleobase can be attached to a deoxyribose or a ribose, while “d” without parentheses indicates that the nucleobase is attached to deoxyribose.

As used herein, a “nucleoside” is a compound comprising a nucleobase moiety and a sugar moiety. Nucleosides include, but are not limited to, naturally occurring nucleosides (as found in DNA and RNA), abasic nucleosides, modified nucleosides, and nucleosides having mimetic bases and/or sugar groups. Nucleosides include nucleosides comprising any variety of substituents. A nucleoside can be a glycoside compound formed through glycosidic linking between a nucleic acid base and a reducing group of a sugar.

An “analog” of a chemical structure, as the term is used herein, refers to a chemical structure that preserves substantial similarity with the parent structure, although it may not be readily derived synthetically from the parent structure. In some embodiments, a nucleotide analog is an unnatural nucleotide. In some embodiments, a nucleoside analog is an unnatural nucleoside. A related chemical structure that is readily derived synthetically from a parent chemical structure is referred to as a “derivative.”

As used herein, “dose-limiting toxicity” (DLT) is defined as an adverse event occurring within Day 1 through Day 29 (inclusive)±1 day of a treatment cycle that was not clearly or incontrovertibly solely related to an extraneous cause and that meets the criteria set forth in Example 2 for DLT.

As used herein, “severe cytokine release syndrome” refers to level 4 or 5 cytokine release syndrome as described in Teachey et al., Cancer Discov. 2016; 6(6); 664-79, the disclosure of which is incorporated herein by reference.

As used herein, “pembrolizumab” refers to the humanized anti-PD-1 antibody marketed under the brand name “Keytruda” by Merck & Co.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### IL-2 Conjugates

Interleukin 2 (IL-2) is a pleiotropic type-1 cytokine whose structure comprises a 15.5 kDa four α-helix bundle. The precursor form of IL-2 is 153 amino acid residues in length, with the first 20 amino acids forming a signal peptide and residues 21-153 forming the mature form. IL-2 is produced primarily by CD4+ T cells post antigen stimulation and to a lesser extent, by CD8+ cells, Natural Killer (NK) cells, and Natural killer T (NKT) cells, activated dendritic cells (DCs), and mast cells. IL-2 signaling occurs through interaction with specific combinations of IL-2 receptor (IL-2R) subunits, IL-2Rα (also known as CD25), IL-2Rβ (also known as CD122), and IL-2Rγ (also known as CD132). Interaction of IL-2 with the IL-2Rα forms the “low-affinity” IL-2 receptor complex with a K_{d} of about 10⁻⁸M. Interaction of IL-2 with IL-2Rβ and IL-2Rγ forms the “intermediate-affinity” IL-2 receptor complex with a K_{d} of about 10⁻⁹M. Interaction of IL-2 with all three subunits, IL-2Rα, IL-2Rβ, and IL-2Rγ, forms the “high-affinity” IL-2 receptor complex with a K_{d} of about >10⁻¹¹ M.

In some instances, IL-2 signaling via the “high-affinity” IL-2Rαβγ complex modulates the activation and proliferation of regulatory T cells. Regulatory T cells, or CD4⁺CD25⁺Foxp3⁺ regulatory T (Treg) cells, mediate maintenance of immune homeostasis by suppression of effector cells such as CD4+ T cells, CD8+ T cells, B cells, NK cells, and NKT cells. In some instances, Treg cells are generated from the thymus (tTreg cells) or are induced from naïve T cells in the periphery (pTreg cells). In some cases, Treg cells are considered as the mediator of peripheral tolerance. Indeed, in one study, transfer of CD25-depleted peripheral CD4+ T cells produced a variety of autoimmune diseases in nude mice, whereas cotransfer of CD4⁺CD25⁺ T cells suppressed the development of autoimmunity (Sakaguchi, et al., “Immunologic self-tolerance maintained by activated T cells expressing IL-2 receptor alpha-chains (CD25),” J. Immunol. 155(3): 1151-1164 (1995), the disclosure of which is incorporated herein by reference). Augmentation of the Treg cell population down-regulates effector T cell proliferation and suppresses autoimmunity and T cell anti-tumor responses.

IL-2 signaling via the “intermediate-affinity” IL-2Rβγ complex modulates the activation and proliferation of CD8⁺ effector T (Teff) cells, NK cells, and NKT cells. CD8⁺ Teff cells (also known as cytotoxic T cells, Tc cells, cytotoxic T lymphocytes, CTLs, T-killer cells, cytolytic T cells, Tcon, or killer T cells) are T lymphocytes that recognize and kill damaged cells, cancerous cells, and pathogen-infected cells. NK and NKT cells are types of lymphocytes that, similar to CD8⁺ Teff cells, target cancerous cells and pathogen-infected cells.

In some instances, IL-2 signaling is utilized to modulate T cell responses and subsequently for treatment of a cancer. For example, IL-2 is administered in a high-dose form to induce expansion of Teff cell populations for treatment of a cancer. However, high-dose IL-2 further leads to concomitant stimulation of Treg cells that dampen anti-tumor immune responses. High-dose IL-2 also induces toxic adverse events mediated by the engagement of IL-2R alpha chain-expressing cells in the vasculature, including type 2 innate immune cells (ILC-2), eosinophils and endothelial cells. This leads to eosinophilia, capillary leak and vascular leak syndrome (VLS).

Adoptive cell therapy enables physicians to effectively harness a patient's own immune cells to fight diseases such as proliferative disease (e.g., cancer) as well as infectious disease. The effect of IL-2 signaling may be further enhanced by the presence of additional agents or methods in combination therapy. For example, programmed cell death protein 1, also known as PD-1 or CD279, is a cell surface receptor expressed on T cells and pro-B cells which plays a role in regulating the immune system's response to the cells of the human body. PD-1 down-regulates the immune system and promotes self-tolerance by suppressing T cell inflammatory activity. This prevents autoimmune diseases but can also prevent the immune system from killing cancer cells. PD-1 guards against autoimmunity through two mechanisms. First, PD-1 promotes apoptosis (programmed cell death) of antigen-specific T-cells in lymph nodes. Second, PD-1 reduces apoptosis in regulatory T cells (anti-inflammatory, suppressive T cells). Pembrolizumab is a humanized anti-PD-1 antibody that can block PD-1, activate the immune system to attack tumors, and is approved for treatment of certain cancers.

Provided herein are methods of treating a cancer in a subject in need thereof, comprising administering to the subject (a) about 8 μg/kg, 16 μg/kg, 24 μg/kg, or 32 μg/kg of an IL-2 conjugate, and (b) pembrolizumab.

In some embodiments, the IL-2 sequence comprises the sequence of SEQ ID NO: 1: PTSSSTKKTQLQLEHLLLDLQMILNGINNYKNPKLTRMLTFKFYMPKKATELKHLQCLE EELKPLEEVLNLAQSKNFHLRPRDLISNINVIVLELKGSETTFMCEYADETATIVEFLNR WITFSQSIISTLT (SEQ ID NO: 1)
wherein the amino acid at position P64 is replaced by the structure of Formula (IA):

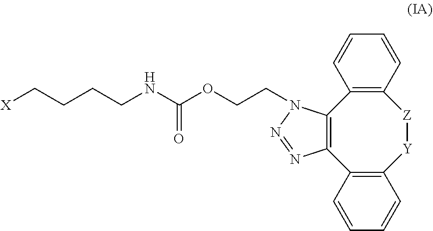

wherein:
Z is CH₂ and Y is

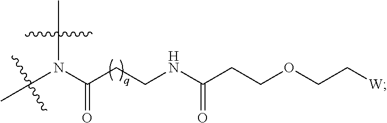

Y is CH₂ and Z is

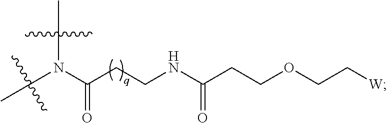

Z is CH₂ and Y is

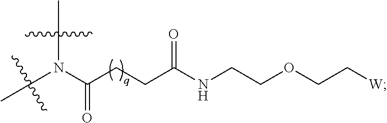

or
Y is CH₂ and Z is

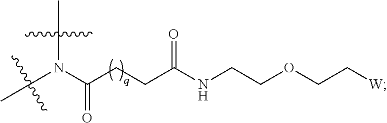

W is a PEG group having an average molecular weight of about 25 kDa-35 kDa;
q is 1, 2, or 3;
X is an L-amino acid having the structure:

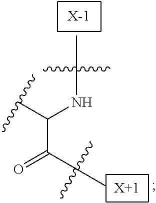 [SMILES: "X-1" ; *NC(*)C(*)=O ; "X+1"]

X−1 indicates the point of attachment to the preceding amino acid residue; and
X+1 indicates the point of attachment to the following amino acid residue.

In any of the embodiments or variations of Formula (IA) described herein, the IL-2 conjugate is a pharmaceutically acceptable salt, solvate, or hydrate. In some embodiments, the IL-2 conjugate is a pharmaceutically acceptable salt. In some embodiments, the IL-2 conjugate is a solvate. In some embodiments, the IL-2 conjugate is a hydrate.

In some embodiments of Formula (IA), Z is CH₂ and Y is

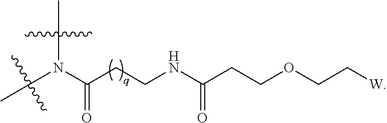

In some embodiments of Formula (IA), Y is CH₂ and Z is

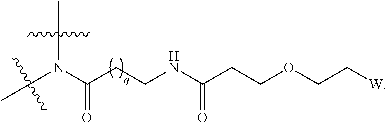

In some embodiments of Formula (IA), Z is CH₂ and Y is

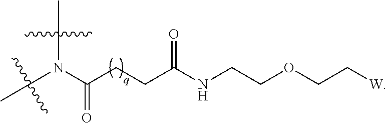

In some embodiments of Formula (IA), Y is CH₂ and Z is

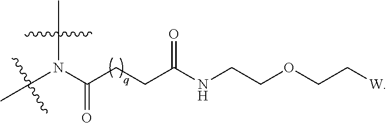

In some embodiments of Formula (IA), q is 1. In some embodiments of Formula (IA), q is 2. In some embodiments of Formula (IA), q is 3.

In some embodiments of Formula (IA), W is a PEG group having an average molecular weight of about 25 kDa. In some embodiments of Formula (IA), W is a PEG group having an average molecular weight of about 30 kDa. In some embodiments of Formula (IA), W is a PEG group having an average molecular weight of about 35 kDa.

In some embodiments, the IL-2 sequence comprises the sequence of SEQ ID NO: 1:

(SEQ ID NO: 1)
PTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGS
ETTFMCEYADETATIVEFLNRWITFSQSIISTLT

wherein the amino acid at position P64 is replaced by the structure of Formula (I):

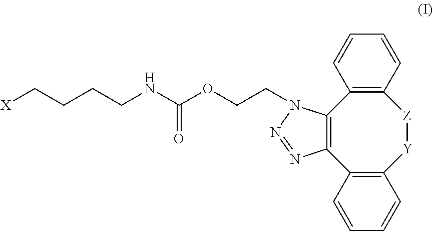

wherein:
Z is CH₂ and Y is

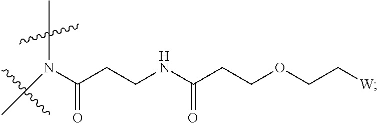

Y is CH₂ and Z is

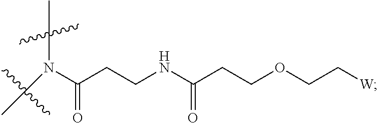

Z is CH₂ and Y is

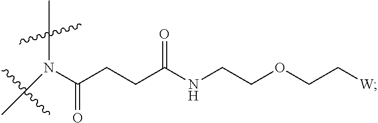

or
Y is CH₂ and Z is

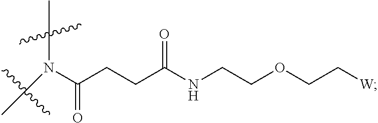

W is a PEG group having an average molecular weight of about 25 kDa-35 kDa;
X is an L-amino acid having the structure:

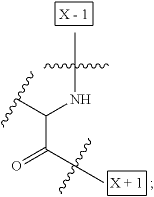 [SMILES: *NC(*)C(*)=O]

X−1 indicates the point of attachment to the preceding amino acid residue; and
X+1 indicates the point of attachment to the following amino acid residue.

In any of the embodiments or variations of Formula (I) described herein, the IL-2 conjugate is a pharmaceutically acceptable salt, solvate, or hydrate. In some embodiments, the IL-2 conjugate is a pharmaceutically acceptable salt. In some embodiments, the IL-2 conjugate is a solvate. In some embodiments, the IL-2 conjugate is a hydrate.

In some embodiments of Formula (I), Z is CH₂ and Y is

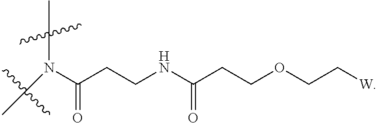

In some embodiments of Formula (I), Y is CH₂ and Z is

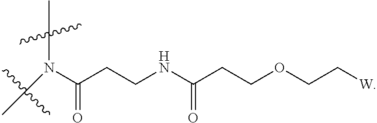

In other embodiments of Formula (I), Z is CH₂ and Y is

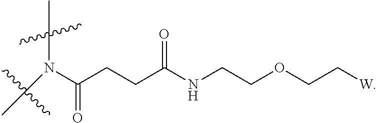

In some embodiments of Formula (I), Y is CH₂ and Z is

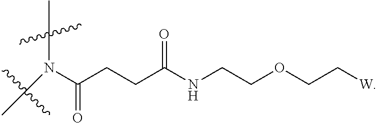

In some embodiments of Formula (I), the PEG group has an average molecular weight of about 30 kDa.

In some embodiments, the IL-2 conjugate comprises the sequence of SEQ ID NO: 2:

(SEQ ID NO: 2)
PTSSSTKKTQLQLEHLLLDLQMILNGINNYKNPKLTRMLTFKFYMPKKA
TELKHLQCLEEELK[AzK L1 PEG30kD]LEEVLNLAQSKNFHLRPRD
LISNINVIVLELKGSETTFMCEYADETATIVEFLNRWITFSQSIISTLT

wherein [AzK_L1_PEG30 kD] is N6-((2-azidoethoxy)-carbonyl)-L-lysine stably-conjugated to PEG via DBCO-mediated click chemistry to form a compound comprising a structure of Formula (IVA) or Formula (VA), wherein q is 1 (such as Formula (IV) or Formula (V)), wherein the PEG group has an average molecular weight of about 25-35 kiloDaltons (e.g., about 30 kDa), capped with a methoxy group. The term “DBCO” means a chemical moiety comprising a dibenzocyclooctyne group, such as comprising the mPEG-DBCO compound illustrated in Schemes 1 and 2 of Example 1.

The ratio of regioisomers generated from the click reaction is about 1:1 or greater than 1:1.

PEGs will typically comprise a number of (OCH₂CH₂) monomers (or (CH₂CH₂O) monomers, depending on how the PEG is defined). In some embodiments, the number of (OCH₂CH₂) monomers (or (CH₂CH₂O) monomers) is such that the average molecular weight of the PEG group is about 30 kDa.

In some instances, the PEG is an end-capped polymer, that is, a polymer having at least one terminus capped with a relatively inert group, such as a lower C₁₋₆ alkoxy group, or a hydroxyl group. In some embodiments, the PEG group is a methoxy-PEG (commonly referred to as mPEG), which is a linear form of PEG wherein one terminus of the polymer is a methoxy (—OCH₃) group, and the other terminus is a hydroxyl or other functional group that can be optionally chemically modified.

In some embodiments, the PEG group is a linear or branched PEG group. In some embodiments, the PEG group is a linear PEG group. In some embodiments, the PEG group is a branched PEG group. In some embodiments, the PEG group is a methoxy PEG group. In some embodiments, the PEG group is a linear or branched methoxy PEG group. In some embodiments, the PEG group is a linear methoxy PEG group. In some embodiments, the PEG group is a branched methoxy PEG group. For example, included within the scope of the present disclosure are IL-2 conjugates comprising a PEG group having a molecular weight of 30,000 Da±3,000 Da, or 30,000 Da±4,500 Da, or 30,000 Da±5,000 Da.

In some embodiments, the IL-2 conjugate comprises the amino acid sequence of SEQ ID NO: 1 in which the amino acid residue P64 is replaced by the structure of Formula (IVA) or Formula (VA), or a mixture of Formula (IVA) and Formula (VA):

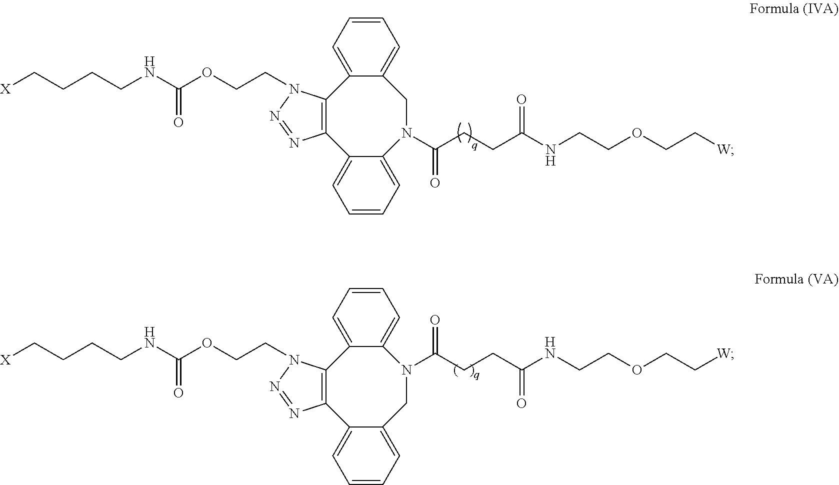

wherein:
W is a PEG group having an average molecular weight of about 25 kDa-35 kDa;
q is 1, 2, or 3; and
X has the structure:

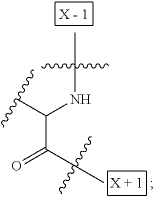 [SMILES: *NC(*)C(*)=O]

X−1 indicates the point of attachment to the preceding amino acid residue; and
X+1 indicates the point of attachment to the following amino acid residue.

In some embodiments of Formula (IVA) or Formula (VA), or a mixture of Formula (IVA) or Formula (VA), q is 1. In some embodiments of Formula (IVA) or Formula (VA), or a mixture of Formula (IVA) or Formula (VA), q is 2. In some embodiments of Formula (IVA) or Formula (VA), or a mixture of Formula (IVA) or Formula (VA), q is 3.

In some embodiments of Formula (IVA) or Formula (VA), or a mixture of Formula (IVA) or Formula (VA), W is a PEG group having an average molecular weight of about 25 kDa. In some embodiments of Formula (IVA) or Formula (VA), or a mixture of Formula (IVA) or Formula (VA), W is a PEG group having an average molecular weight of about 30 kDa. In some embodiments of Formula (IVA) or Formula (VA), or a mixture of Formula (IVA) or Formula (VA), W is a PEG group having an average molecular weight of about 35 kDa.

In any of the embodiments described herein, the structure of Formula (IA) has the structure of Formula (IVA) or Formula (VA), or is a mixture of Formula (IVA) and Formula (VA). In some embodiments, the structure of Formula (IA) has the structure of Formula (IVA). In some embodiments, the structure of Formula (IA) has the structure of Formula (VA). In some embodiments, the structure of Formula (IA) is a mixture of Formula (IVA) and Formula (VA).

In some embodiments, the IL-2 conjugate comprises the amino acid sequence of SEQ ID NO: 1 in which the amino acid residue P64 is replaced by the structure of Formula (IV) or Formula (V), or a mixture of Formula (IV) and Formula (V):

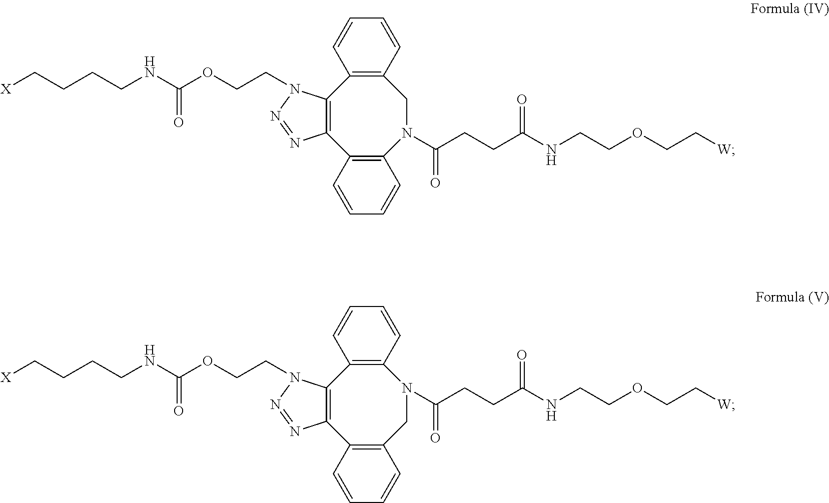

wherein:
W is a PEG group having an average molecular weight of about 25 kDa-35 kDa; and
X has the structure:

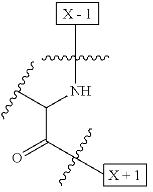

X−1 indicates the point of attachment to the preceding amino acid residue; and
X+1 indicates the point of attachment to the following amino acid residue.

In some embodiments of Formula (IV) or Formula (V), or a mixture of Formula (IV) and Formula (V), the PEG group has an average molecular weight of about 30 kDa.

In any of the embodiments described herein, the structure of Formula (IA) has the structure of Formula (IV) or Formula (V), or is a mixture of Formula (IV) and Formula (V). In some embodiments, the structure of Formula (IA) has the structure of Formula (IV). In some embodiments, the structure of Formula (IA) has the structure of Formula (V). In some embodiments, the structure of Formula (IA) is a mixture of Formula (IV) and Formula (V).

In some embodiments, the IL-2 conjugate comprises the amino acid sequence of SEQ ID NO: 1 in which the amino acid residue P64 is replaced by the structure of Formula (XIIA) or Formula (XIIIA), or a mixture of Formula (XIIA) and Formula (XIIIA)

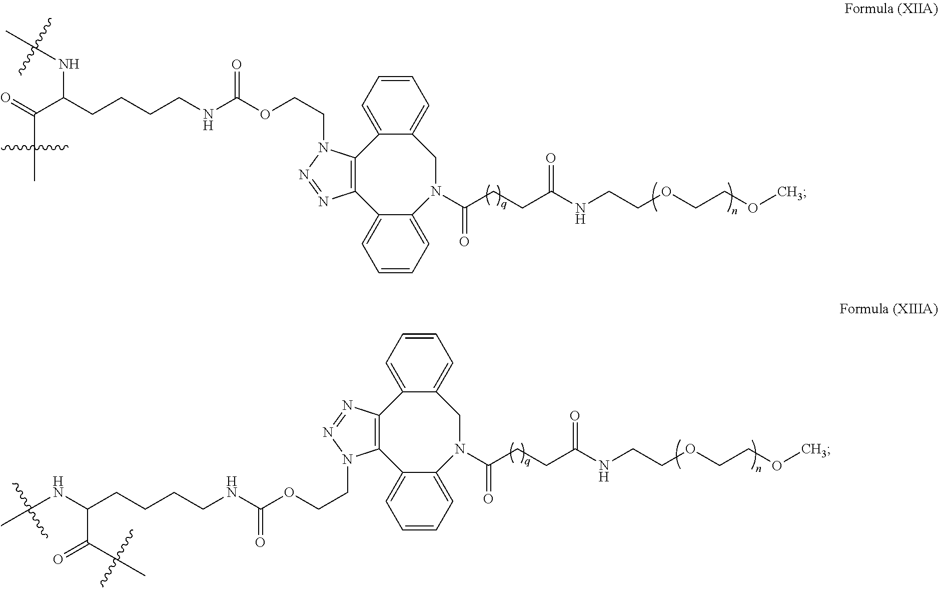

wherein:
n is an integer such that —(OCH₂CH₂)ₙ—OCH₃ has a molecular weight of about 25 kDa-35 kDa;
q is 1, 2, or 3; and
the wavy lines indicate covalent bonds to amino acid residues within SEQ ID NO: 1 that are not replaced.

In some embodiments of Formula (XIIA) or Formula (XIIIA), or a mixture of Formula (XIIA) and Formula (XIIIA), q is 1. In some embodiments of Formula (XIIA) or Formula (XIIIA), or a mixture of Formula (XIIA) and Formula (XIIIA), q is 2. In some embodiments of Formula (XIIA) or Formula (XIIIA), or a mixture of Formula (XIIA) and Formula (XIIIA), q is 3.

In some embodiments of Formula (XIIA) or Formula (XIIIA), or a mixture of Formula (XIIA) and Formula (XIIIA), n is an integer such that —(OCH₂CH₂)ₙ—OCH₃ has a molecular weight of about 30 kDa.

In any of the embodiments described herein, the structure of Formula (IA) has the structure of Formula (XIIA) or Formula (XIIIA), or is a mixture of Formula (XIIA) and Formula (XIIIA) In some embodiments, the structure of Formula (IA) has the structure of Formula (XIIA). In some embodiments, the structure of Formula (IA) has the structure of Formula (XIIIA) In some embodiments, the structure of Formula (IA) is a mixture of Formula (XIIA) and Formula (XIIIA).

In some embodiments, the IL-2 conjugate comprises the amino acid sequence of SEQ ID NO: 1 in which the amino acid residue P64 is replaced by the structure of Formula (XII) or Formula (XIII), or a mixture of Formula (XII) and Formula (XIII):

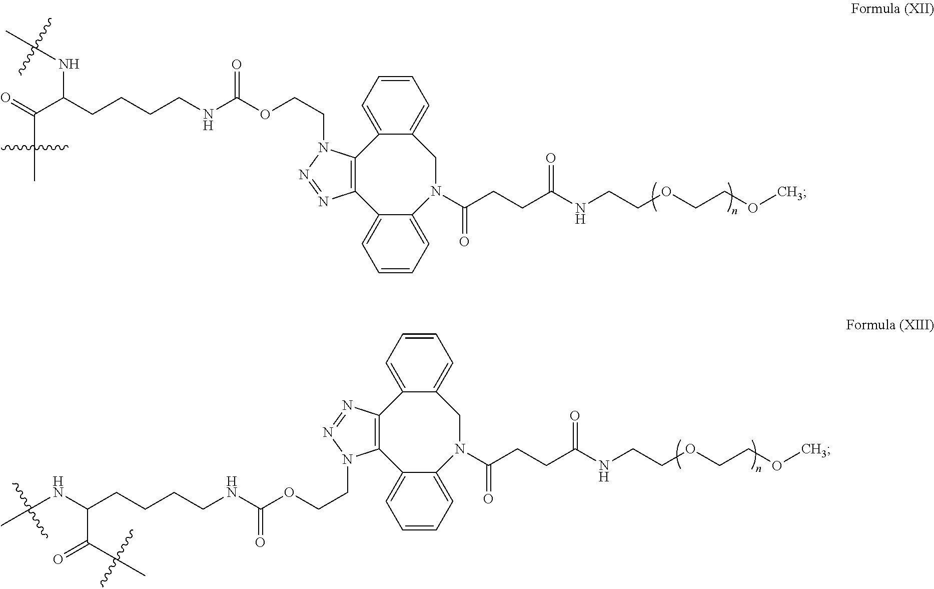

wherein:
n is an integer such that —(OCH₂CH₂)ₙ—OCH₃ has a molecular weight of about 25 kDa-35 kDa; and
the wavy lines indicate covalent bonds to amino acid residues within SEQ ID NO: 1 that are not replaced.

In some embodiments of Formula (XII) or Formula (XIII), or a mixture of Formula (XII) and Formula (XIII), n is an integer such that —(OCH₂CH₂)ₙ—OCH₃ has a molecular weight of about 30 kDa.

In any of the embodiments described herein, the structure of Formula (IA) has the structure of Formula (XII) or Formula (XIII), or is a mixture of Formula (XII) and Formula (XIII). In some embodiments, the structure of Formula (IA) has the structure of Formula (XII). In some embodiments, the structure of Formula (IA) has the structure of Formula (XIII). In some embodiments, the structure of Formula (IA) is a mixture of Formula (XII) and Formula (XIII).

In some embodiments, the IL-2 conjugate comprises the amino acid sequence of SEQ ID NO: 1 in which the amino acid residue P64 is replaced by the structure of Formula (XIV) or Formula (XV), or a mixture of Formula (XIV) and Formula (XV):

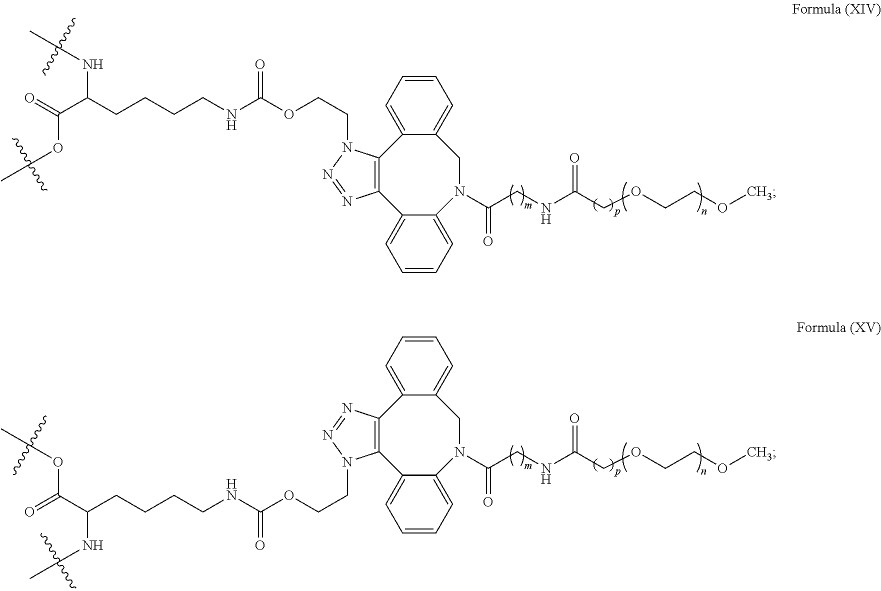

wherein:
m is an integer from 0 to 20;
p is an integer from 0 to 20;
n is an integer such that the PEG group has an average molecular weight of about 25 kDa-35 kDa; and
the wavy lines indicate covalent bonds to amino acid residues within SEQ ID NO: 1 that are not replaced.

In some embodiments of Formula (XIV) or Formula (XV), or a mixture of Formula (XIV) and Formula (XV), n is an integer such that the PEG group has an average molecular weight of about 30 kDa. In some embodiments of Formula (XIV) or (XV), or a mixture of (XIV) and (XV), n is an integer such that the PEG group has an average molecular weight of about 25 kDa. In some embodiments of Formula (XIV) or (XV), or a mixture of (XIV) and (XV), n is an integer such that the PEG group has an average molecular weight of about 35 kDa.

In some embodiments, m is an integer from 0 to 15. In some embodiments, m is an integer from 0 to 10. In some embodiments, m is an integer from 0 to 5. In some embodiments, m is an integer from 1 to 3. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10.

In some embodiments, p is an integer from 0 to 15. In some embodiments, p is an integer from 0 to 10. In some embodiments, p is an integer from 0 to 5. In some embodiments, p is an integer from 1 to 3. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10.

In any of the embodiments described herein, the structure of Formula (IA) has the structure of Formula (XIV) or Formula (XV), or is a mixture of Formula (XIV) and Formula (XV). In some embodiments, the structure of Formula (IA) has the structure of Formula (XIV). In some embodiments, the structure of Formula (IA) has the structure of Formula (XV). In some embodiments, the structure of Formula (IA) is a mixture of Formula (XIV) and Formula (XV).

In some embodiments, the IL-2 conjugate comprises the amino acid sequence of SEQ ID NO: 1 in which the amino acid residue P64 is replaced by the structure of Formula (XVI) or Formula (XVII), or a mixture of Formula (XVI) and Formula (XVII):

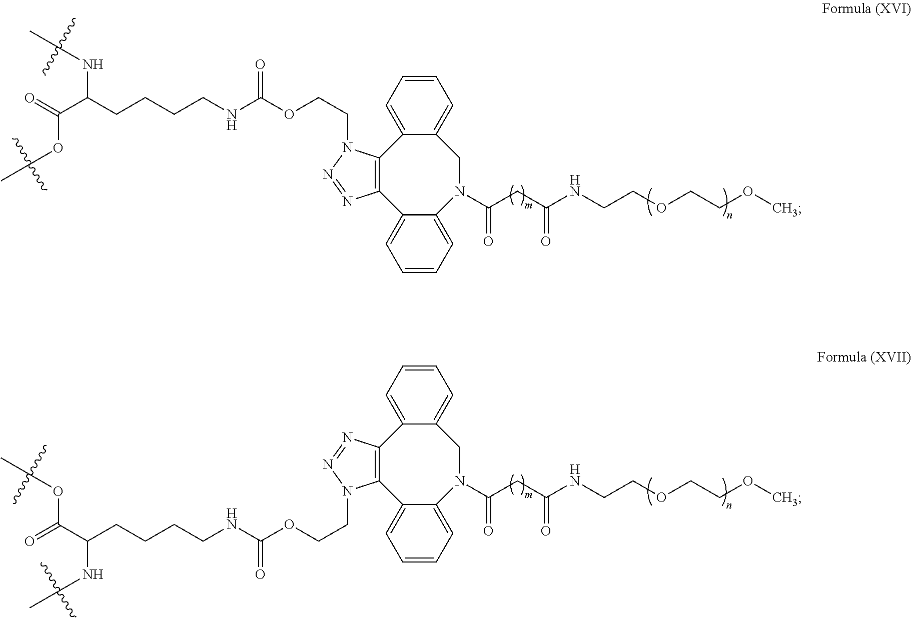

wherein:
m is an integer from 0 to 20;
n is an integer such that the PEG group has an average molecular weight of about 25 kDa-35 kDa; and
the wavy lines indicate covalent bonds to amino acid residues within SEQ ID NO: 1 that are not replaced.

In some embodiments of Formula (XVI) or Formula (XVII), or a mixture of Formula (XVI) and Formula (XVII), n is an integer such that the PEG group has an average molecular weight of about 30 kDa. In some embodiments of Formula (XVI) or Formula (XVII), or a mixture of Formula (XVI) and Formula (XVII), n is an integer such that the PEG group has an average molecular weight of about 25 kDa. In some embodiments of Formula (XVI) or Formula (XVII), or a mixture of Formula (XVI) and Formula (XVII), n is an integer such that the PEG group has an average molecular weight of about 35 kDa.

In some embodiments, m is an integer from 0 to 15. In some embodiments, m is an integer from 0 to 10. In some embodiments, m is an integer from 0 to 5. In some embodiments, m is an integer from 1 to 3. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10.

In any of the embodiments described herein, the structure of Formula (IA) has the structure of Formula (XVI) or Formula (XVII), or is a mixture of Formula (XVI) and Formula (XVII). In some embodiments, the structure of Formula (IA) has the structure of Formula (XVI). In some embodiments, the structure of Formula (IA) has the structure of Formula (XVII). In some embodiments, the structure of Formula (IA) is a mixture of Formula (XVI) and Formula (XVII).

In some embodiments of Formula (IA) or any variation thereof, the IL-2 conjugate has an in vivo half-life of about 10 hours.

### Conjugation Chemistry

In some embodiments, the IL-2 conjugates described herein can be prepared by a conjugation reaction comprising a 1,3-dipolar cycloaddition reaction. In some embodiments, the 1,3-dipolar cycloaddition reaction comprises reaction of an azide and an alkyne (“Click” reaction). In some embodiments, a conjugation reaction described herein comprises the reaction outlined in Scheme I, wherein X is an unnatural amino acid at position P64 of SEQ ID NO: 1.

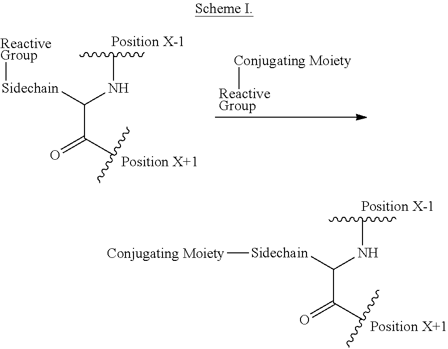

In some embodiments, the conjugating moiety comprises a PEG group as described herein. In some embodiments, a reactive group comprises an alkyne or azide.

In some embodiments, a conjugation reaction described herein comprises the reaction outlined in Scheme II, wherein X is an unnatural amino acid at position P64 of SEQ ID NO: 1.

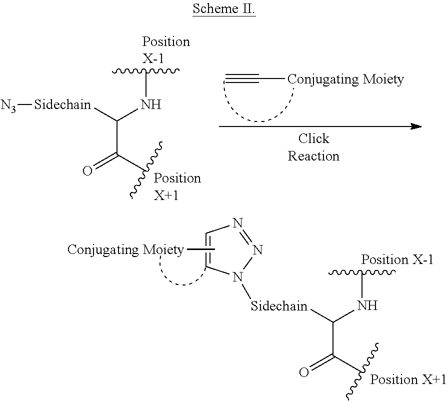

In some embodiments, a conjugation reaction described herein comprises the reaction outlined in Scheme III, wherein X is an unnatural amino acid at position P64 of SEQ ID NO: 1.

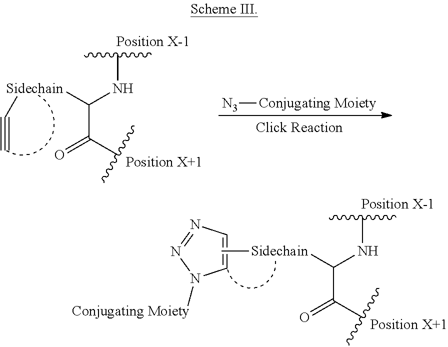

In some embodiments a conjugation reaction described herein comprises the reaction outlined in Scheme IV, wherein X is an unnatural amino acid at position P64 of SEQ ID NO: 1.

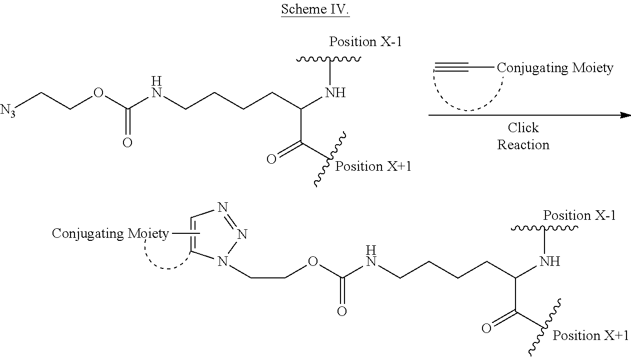

In some embodiments, a conjugation reaction described herein comprises a cycloaddition reaction between an azide moiety, such as that contained in a protein containing an amino acid residue derived from N6-((2-azidoethoxy)-carbonyl)-L-lysine (AzK), and a strained cycloalkyne, such as that derived from DBCO, which is a chemical moiety comprising a dibenzocyclooctyne group. PEG groups comprising a DBCO moiety are commercially available or may be prepared by methods known to those of ordinary skill in the art. Exemplary reactions are shown in Schemes V and VI.

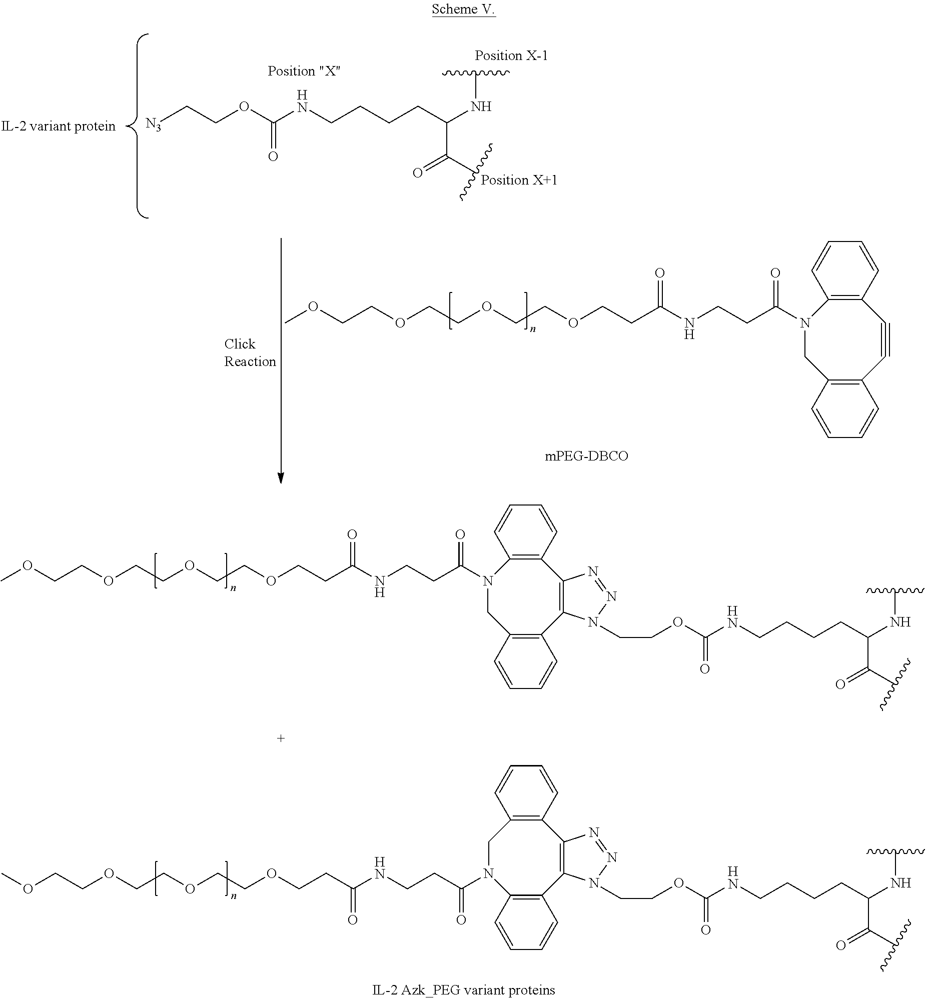

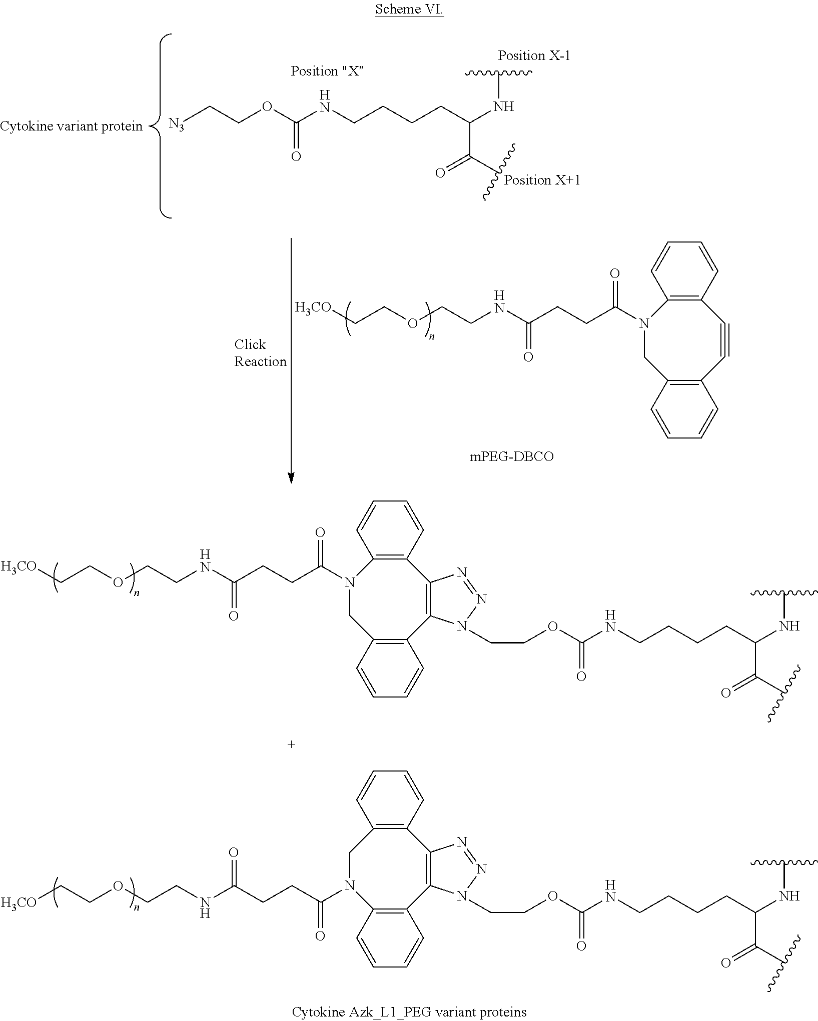

Conjugation reactions such as a click reaction described herein may generate a single regioisomer, or a mixture of regioisomers. In some instances the ratio of regioisomers is about 1:1. In some instances the ratio of regioisomers is about 2:1. In some instances the ratio of regioisomers is about 1.5:1. In some instances the ratio of regioisomers is about 1.2:1. In some instances the ratio of regioisomers is about 1.1:1. In some instances the ratio of regioisomers is greater than 1:1.

### IL-2 Polypeptide Production

In some instances, the IL-2 conjugates described herein, either containing a natural amino acid mutation or an unnatural amino acid mutation, are generated recombinantly or are synthesized chemically. In some instances, IL-2 conjugates described herein are generated recombinantly, for example, either by a host cell system, or in a cell-free system.

In some instances, IL-2 conjugates are generated recombinantly through a host cell system. In some cases, the host cell is a eukaryotic cell (e.g., mammalian cell, insect cells, yeast cells or plant cell) or a prokaryotic cell (e.g., Gram-positive bacterium or a Gram-negative bacterium). In some cases, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293A cell line, 293FT cell line, 293F cells, 293 H cells, A549 cells, MDCK cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some embodiments, a eukaryotic host cell is an insect host cell. Exemplary insect host cells include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris (K. phaffii) yeast strains such as GS115, KM71H, SMD1168, SMD1168H, and X-33, and Saccharomyces cerevisiae yeast strain such as INVSc1.

In some embodiments, a eukaryotic host cell is a plant host cell. In some instances, the plant cells comprise a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, suitable polynucleic acid molecules or vectors for the production of an IL-2 polypeptide described herein include any suitable vectors derived from either a eukaryotic or prokaryotic source. Exemplary polynucleic acid molecules or vectors include vectors from bacteria (e.g., E. coli), insects, yeast (e.g., Pichia pastoris, K. phaffii), algae, or mammalian source. Bacterial vectors include, for example, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris (K. phaffii) vector, pGAPZA, B, & C Pichia pastoris (K. phaffii) vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is used for the production of an IL-2 polypeptide described herein. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, Archaea, or HeLa cells. Exemplary cell-free systems include E. coli S30 Extract system, E. coli T7 S30 system, or PURExpress®, XpressCF, and XpressCF+.

Cell-free translation systems variously comprise components such as plasmids, mRNA, DNA, tRNAs, synthetases, release factors, ribosomes, chaperone proteins, translation initiation and elongation factors, natural and/or unnatural amino acids, and/or other components used for protein expression. Such components are optionally modified to improve yields, increase synthesis rate, increase protein product fidelity, or incorporate unnatural amino acids. In some embodiments, cytokines described herein are synthesized using cell-free translation systems described in U.S. Pat. No. 8,778,631; US 2017/0283469; US 2018/0051065; US 2014/0315245; or U.S. Pat. No. 8,778,631, the disclosure of each of which is incorporated herein by reference. In some embodiments, cell-free translation systems comprise modified release factors, or even removal of one or more release factors from the system. In some embodiments, cell-free translation systems comprise a reduced protease concentration. In some embodiments, cell-free translation systems comprise modified tRNAs with re-assigned codons used to code for unnatural amino acids. In some embodiments, the synthetases described herein for the incorporation of unnatural amino acids are used in cell-free translation systems. In some embodiments, tRNAs are pre-loaded with unnatural amino acids using enzymatic or chemical methods before being added to a cell-free translation system. In some embodiments, components for a cell-free translation system are obtained from modified organisms, such as modified bacteria, yeast, or other organism.

In some embodiments, an IL-2 polypeptide is generated as a circularly permuted form, either via an expression host system or through a cell-free system.

### Production of Cytokine Polypeptide Comprising an Unnatural Amino Acid

An orthogonal or expanded genetic code can be used in the present disclosure, in which one or more specific codons present in the nucleic acid sequence of an IL-2 polypeptide are allocated to encode the unnatural amino acid so that it can be genetically incorporated into the IL-2 by using an orthogonal tRNA synthetase/tRNA pair. The orthogonal tRNA synthetase/tRNA pair is capable of charging a tRNA with an unnatural amino acid and is capable of incorporating that unnatural amino acid into the polypeptide chain in response to the codon.

In some instances, the codon is the codon amber, ochre, opal or a quadruplet codon. In some cases, the codon corresponds to the orthogonal tRNA which will be used to carry the unnatural amino acid. In some cases, the codon is amber. In other cases, the codon is an orthogonal codon.

In some instances, the codon is a quadruplet codon, which can be decoded by an orthogonal ribosome ribo-Q1. In some cases, the quadruplet codon is as illustrated in Neumann, et al., “Encoding multiple unnatural amino acids via evolution of a quadruplet-decoding ribosome,” Nature, 464(7287): 441-444 (2010), the disclosure of which is incorporated herein by reference.

In some instances, a codon used in the present disclosure is a recoded codon, e.g., a synonymous codon or a rare codon that is replaced with alternative codon. In some cases, the recoded codon is as described in Napolitano, et al., “Emergent rules for codon choice elucidated by editing rare arginine codons in Escherichia coli,” PNAS, 113(38): E5588-5597 (2016), the disclosure of which is incorporated herein by reference. In some cases, the recoded codon is as described in Ostrov et al., “Design, synthesis, and testing toward a 57-codon genome,” Science 353(6301): 819-822 (2016), the disclosure of which is incorporated herein by reference.

In some instances, unnatural nucleic acids are utilized leading to incorporation of one or more unnatural amino acids into the IL-2. Exemplary unnatural nucleic acids include, but are not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; WO 99/62923; Kandimalla et al., (2001) Bioorg. Med. Chem. 9:807-813; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; and Sanghvi, Chapter 15, Antisense Research and Applications, Crooke and Lebleu Eds., CRC Press, 1993, 273-288. Additional base modifications can be found, for example, in U.S. Pat. No. 3,687,808; Englisch et al., Angewandte Chemie, International Edition, 1991, 30, 613; and Sanghvi, Chapter 15, Antisense Research and Applications, pages 289-302, Crooke and Lebleu ed., CRC Press, 1993; the disclosure of each of which is incorporated herein by reference.

Unnatural nucleic acids comprising various heterocyclic bases and various sugar moieties (and sugar analogs) are available in the art, and the nucleic acids in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo [2.3-d] pyrimidin-5-yl, 2-amino-4-oxopyrolo [2, 3-d]pyrimidin-5-yl, 2-amino-4-oxopyrrolo [2.3-d] pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050; the disclosure of each of which is incorporated herein by reference.

In some embodiments, unnatural nucleic acids include 2′,3′-dideoxy-2′,3′-didehydro-nucleosides (PCT/US2002/006460), 5′-substituted DNA and RNA derivatives (PCT/US2011/033961; Saha et al., J. Org Chem., 1995, 60, 788-789; Wang et al., Bioorganic & Medicinal Chemistry Letters, 1999, 9, 885-890; and Mikhailov et al., Nucleosides & Nucleotides, 1991, 10(1-3), 339-343; Leonid et al., 1995, 14(3-5), 901-905; and Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al., Nucleosides Nucleotides & Nucleic Acids, 2004, 23 (1 & 2), 317-337); the disclosure of each of which is incorporated herein by reference.

In some embodiments, unnatural nucleic acids include modifications at the 5′-position and the 2′-position of the sugar ring (PCT/US94/02993), such as 5′-CH₂-substituted 2′-O-protected nucleosides (Wu et al., Helvetica Chimica Acta, 2000, 83, 1127-1143 and Wu et al., Bioconjugate Chem. 1999, 10, 921-924). In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)—CH₃ (Mesmaeker et al., Synlett, 1997, 1287-1290). Unnatural nucleic acids can include 2′-substituted 5′-CH₂ (or O) modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19(8), 1029-1033). Unnatural nucleic acids can include 5′-phosphonate deoxyribonucleoside monomers and dimers having a 5′-phosphate group (Nawrot et al., Oligonucleotides, 2006, 16(1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105(26), 7638-7640; and Hampton et al., J. Am. Chem. Soc, 1973, 95(13), 4404-4414). The disclosure of each reference listed in this paragraph is incorporated herein by reference.

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids (BNA); replacement of the ribosyl ring oxygen atom with S, N(R), or C(R₁)(R₂) (R═H, C₁-C₁₂ alkyl or a protecting group); and combinations thereof. Examples of chemically modified sugars can be found in WO2008/101157, US2005/0130923, and WO2007/134181, the disclosure of each of which is incorporated herein by reference.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]ₘ CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)n CH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,700,920, the disclosure of each of which is incorporated herein by reference.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof (see, U.S. Pat. No. 7,399,845); 4′-C(CH₃)(CH₃)—O-2′ and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393. The disclosure of each reference listed in this paragraph is incorporated herein by reference.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithionate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24: 1841-1848; Chaturvedi et al., 1996, Nucleic Acids Res. 24:2318-2323; Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, N.Y.; Zon, 1993, “Oligonucleoside Phosphorothioates” in Protocols for Oligonucleotides and Analogs, Synthesis and Properties, Humana Press, pp. 165-190; Miller et al., 1971, JACS 93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8: 1157-1179; the disclosure of each of which is incorporated herein by reference.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179, the disclosure of which is incorporated herein by reference). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM5OJ, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S—H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941. The disclosure of each reference listed in this paragraph is incorporated herein by reference.

In some cases, the unnatural nucleic acids further form unnatural base pairs. Exemplary unnatural nucleotides capable of forming an unnatural DNA or RNA base pair (UBP) under conditions in vivo includes, but is not limited to, TAT1, dTAT1, 5FM, d5FM, TPT3, dTPT3, 5SICS, d5SICS, NaM, dNaM, CNMO, dCNMO, and combinations thereof. In some embodiments, unnatural nucleotides include:

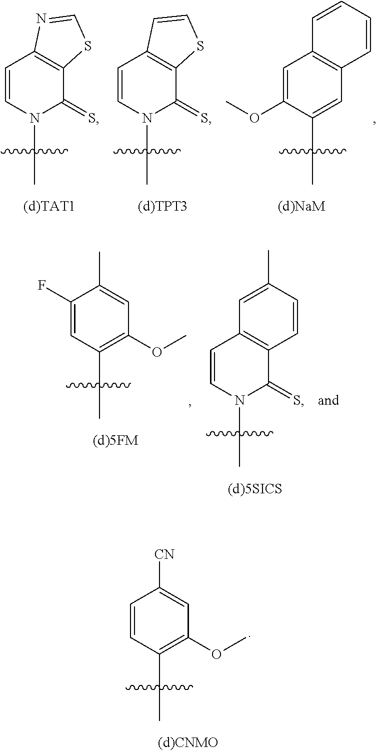

Exemplary unnatural base pairs include: (d)TPT3-(d)NaM; (d)5SICS-(d)NaM; (d)CNMO-(d)TAT1; (d)NaM-(d)TAT1; (d)CNMO-(d)TPT3; and (d)5FM-(d)TAT1.

Other examples of unnatural nucleotides capable of forming unnatural UBPs that may be used to prepare the IL-2 conjugates disclosed herein may be found in Dien et al., J Am Chem Soc., 2018, 140:16115-16123; Feldman et al., J Am Chem Soc, 2017, 139:11427-11433; Ledbetter et al., J Am Chem Soc., 2018, 140:758-765; Dhami et al., Nucleic Acids Res. 2014, 42:10235-10244; Malyshev et al., Nature, 2014, 509:385-388; Betz et al., J Am Chem Soc., 2013, 135:18637-18643; Lavergne et al., J Am Chem Soc. 2013, 135:5408-5419; and Malyshev et al. Proc Natl Acad Sci USA, 2012, 109:12005-12010; the disclosure of each of which is incorporated herein by reference. In some embodiments, unnatural nucleotides include:

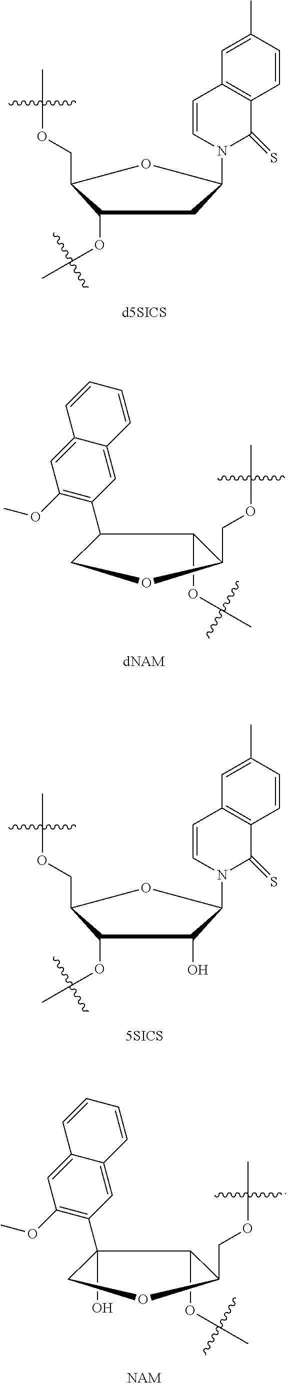

In some embodiments, the unnatural nucleotides that may be used to prepare the IL-2 conjugates disclosed herein may be derived from a compound of the formula

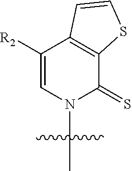

wherein R₂ is selected from the group consisting of hydrogen, alkyl, alkenyl, alkynyl, methoxy, methanethiol, methaneseleno, halogen, cyano, and azido; and

the wavy line indicates a bond to a ribosyl or 2′-deoxyribosyl, wherein the 5′-hydroxy group of the ribosyl or 2′-deoxyribosyl moiety is in free form, is connected to a monophosphate, diphosphate, triphosphate, α-thiotriphosphate, β-thiotriphosphate, or γ-thiotriphosphate group, or is included in an RNA or a DNA or in an RNA analog or a DNA analog.

In some embodiments, the unnatural nucleotides that may be used to prepare the IL-2 conjugates disclosed herein may be derived from a compound of the Formula

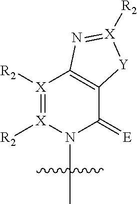

wherein:
each X is independently carbon or nitrogen;
R₂ is absent when X is nitrogen, and is present when X is carbon and is independently hydrogen, alkyl, alkenyl, alkynyl, methoxy, methanethiol, methaneseleno, halogen, cyano, or azide;
Y is sulfur, oxygen, selenium, or secondary amine;
E is oxygen, sulfur, or selenium; and
the wavy line indicates a point of bonding to a ribosyl, deoxyribosyl, or dideoxyribosyl moiety or an analog thereof, wherein the ribosyl, deoxyribosyl, or dideoxyribosyl moiety or analog thereof is in free form, is connected to a mono-phosphate, diphosphate, triphosphate, α-thiotriphosphate, β-thiotriphosphate, or γ-thiotriphosphate group, or is included in an RNA or a DNA or in an RNA analog or a DNA analog.

In some embodiments, each X is carbon. In some embodiments, at least one X is carbon. In some embodiments, one X is carbon. In some embodiments, at least two X are carbon. In some embodiments, two X are carbon. In some embodiments, at least one X is nitrogen. In some embodiments, one X is nitrogen. In some embodiments, at least two X are nitrogen. In some embodiments, two X are nitrogen.

In some embodiments, Y is sulfur. In some embodiments, Y is oxygen. In some embodiments, Y is selenium. In some embodiments, Y is a secondary amine.

In some embodiments, E is sulfur. In some embodiments, E is oxygen. In some embodiments, E is selenium.

In some embodiments, R₂ is present when X is carbon. In some embodiments, R² is absent when X is nitrogen. In some embodiments, each R₂, where present, is hydrogen. In some embodiments, R₂ is alkyl, such as methyl, ethyl, or propyl. In some embodiments, R₂ is alkenyl, such as —CH₂═CH₂. In some embodiments, R₂ is alkynyl, such as ethynyl. In some embodiments, R₂ is methoxy. In some embodiments, R₂ is methanethiol. In some embodiments, R₂ is methaneseleno. In some embodiments, R₂ is halogen, such as chloro, bromo, or fluoro. In some embodiments, R₂ is cyano. In some embodiments, R₂ is azide.

In some embodiments, E is sulfur, Y is sulfur, and each X is independently carbon or nitrogen. In some embodiments, E is sulfur, Y is sulfur, and each X is carbon.

In some embodiments, the unnatural nucleotides that may be used to prepare the IL-2 conjugates disclosed herein may be derived from

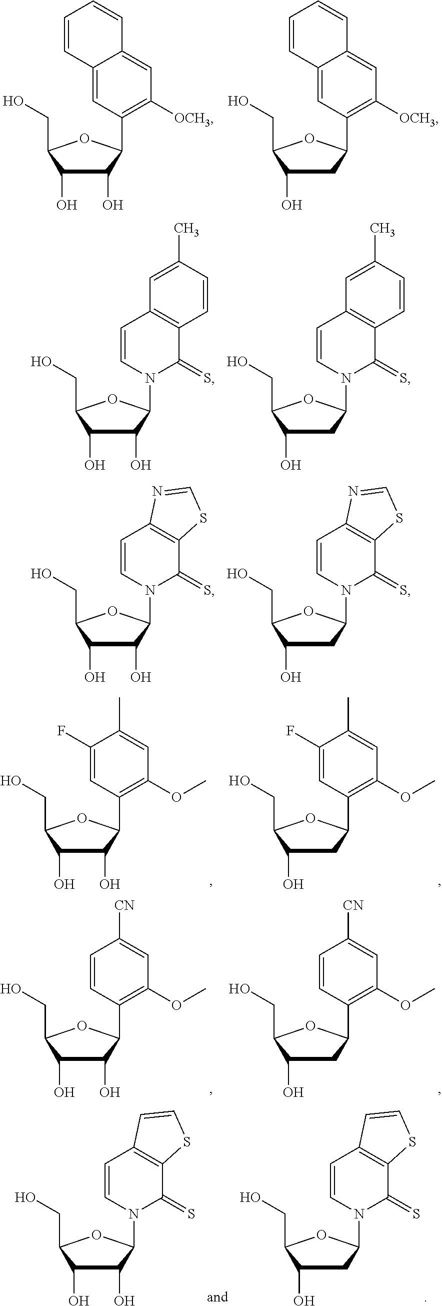

In some embodiments, the unnatural nucleotides that may be used to prepare the IL-2 conjugates disclosed herein include

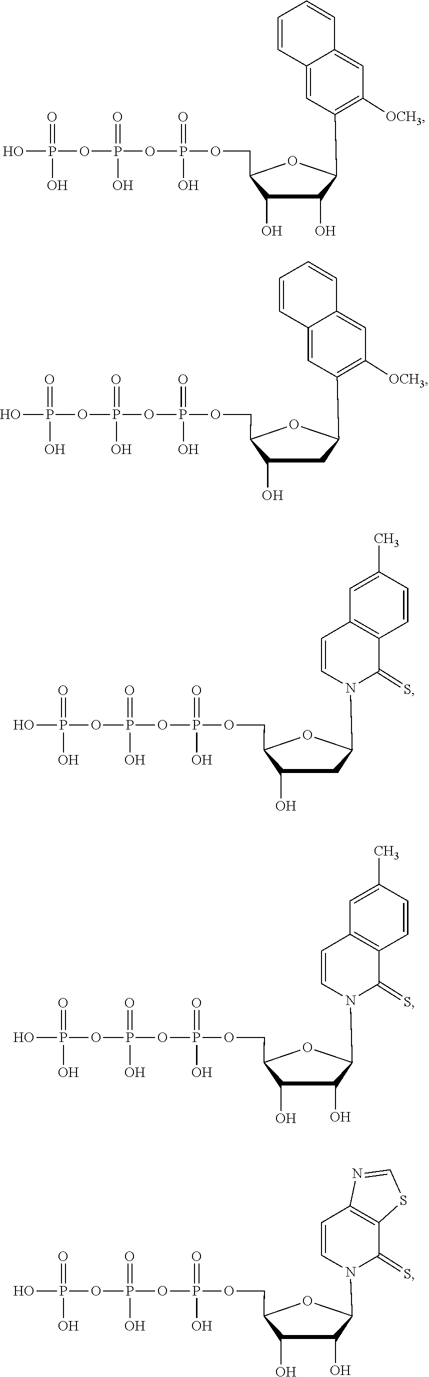

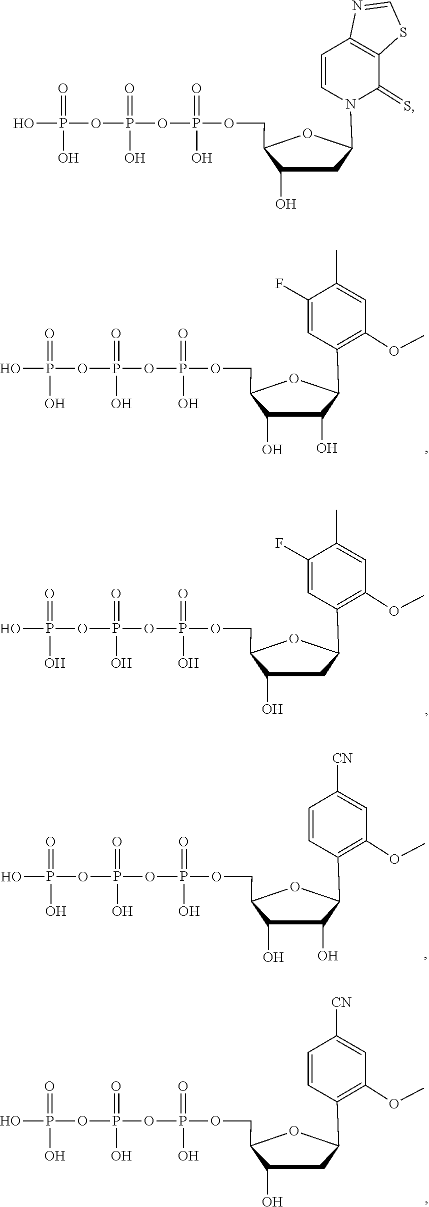

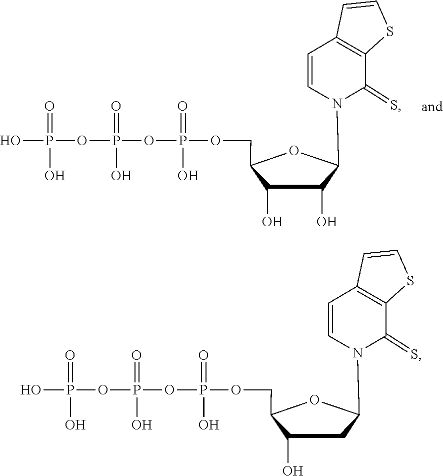

or salts thereof.

In some embodiments, an unnatural base pair generate an unnatural amino acid described in Dumas et al., “Designing logical codon reassignment—Expanding the chemistry in biology,” Chemical Science, 6: 50-69 (2015), the disclosure of which is incorporated herein by reference.

In some embodiments, the unnatural amino acid is incorporated into the cytokine (e.g., the IL polypeptide) by a synthetic codon comprising an unnatural nucleic acid. In some instances, the unnatural amino acid is incorporated into the cytokine by an orthogonal, modified synthetase/tRNA pair. Such orthogonal pairs comprise an unnatural synthetase that is capable of charging the unnatural tRNA with the unnatural amino acid, while minimizing charging of a) other endogenous amino acids onto the unnatural tRNA and b) unnatural amino acids onto other endogenous tRNAs. Such orthogonal pairs comprise tRNAs that are capable of being charged by the unnatural synthetase, while avoiding being charged with a) other endogenous amino acids by endogenous synthetases. In some embodiments, such pairs are identified from various organisms, such as bacteria, yeast, Archaea, or human sources. In some embodiments, an orthogonal synthetase/tRNA pair comprises components from a single organism. In some embodiments, an orthogonal synthetase/tRNA pair comprises components from two different organisms. In some embodiments, an orthogonal synthetase/tRNA pair comprising components that prior to modification, promote translation of two different amino acids. In some embodiments, an orthogonal synthetase is a modified alanine synthetase. In some embodiments, an orthogonal synthetase is a modified arginine synthetase. In some embodiments, an orthogonal synthetase is a modified asparagine synthetase. In some embodiments, an orthogonal synthetase is a modified aspartic acid synthetase. In some embodiments, an orthogonal synthetase is a modified cysteine synthetase. In some embodiments, an orthogonal synthetase is a modified glutamine synthetase. In some embodiments, an orthogonal synthetase is a modified glutamic acid synthetase. In some embodiments, an orthogonal synthetase is a modified alanine glycine. In some embodiments, an orthogonal synthetase is a modified histidine synthetase. In some embodiments, an orthogonal synthetase is a modified leucine synthetase. In some embodiments, an orthogonal synthetase is a modified isoleucine synthetase. In some embodiments, an orthogonal synthetase is a modified lysine synthetase. In some embodiments, an orthogonal synthetase is a modified methionine synthetase. In some embodiments, an orthogonal synthetase is a modified phenylalanine synthetase. In some embodiments, an orthogonal synthetase is a modified proline synthetase. In some embodiments, an orthogonal synthetase is a modified serine synthetase. In some embodiments, an orthogonal synthetase is a modified threonine synthetase. In some embodiments, an orthogonal synthetase is a modified tryptophan synthetase. In some embodiments, an orthogonal synthetase is a modified tyrosine synthetase. In some embodiments, an orthogonal synthetase is a modified valine synthetase. In some embodiments, an orthogonal synthetase is a modified phosphoserine synthetase. In some embodiments, an orthogonal tRNA is a modified alanine tRNA. In some embodiments, an orthogonal tRNA is a modified arginine tRNA. In some embodiments, an orthogonal tRNA is a modified asparagine tRNA. In some embodiments, an orthogonal tRNA is a modified aspartic acid tRNA. In some embodiments, an orthogonal tRNA is a modified cysteine tRNA. In some embodiments, an orthogonal tRNA is a modified glutamine tRNA. In some embodiments, an orthogonal tRNA is a modified glutamic acid tRNA. In some embodiments, an orthogonal tRNA is a modified alanine glycine. In some embodiments, an orthogonal tRNA is a modified histidine tRNA. In some embodiments, an orthogonal tRNA is a modified leucine tRNA. In some embodiments, an orthogonal tRNA is a modified isoleucine tRNA. In some embodiments, an orthogonal tRNA is a modified lysine tRNA. In some embodiments, an orthogonal tRNA is a modified methionine tRNA. In some embodiments, an orthogonal tRNA is a modified phenylalanine tRNA. In some embodiments, an orthogonal tRNA is a modified proline tRNA. In some embodiments, an orthogonal tRNA is a modified serine tRNA. In some embodiments, an orthogonal tRNA is a modified threonine tRNA. In some embodiments, an orthogonal tRNA is a modified tryptophan tRNA. In some embodiments, an orthogonal tRNA is a modified tyrosine tRNA. In some embodiments, an orthogonal tRNA is a modified valine tRNA. In some embodiments, an orthogonal tRNA is a modified phosphoserine tRNA.

In some embodiments, the unnatural amino acid is incorporated into the cytokine (e.g., the IL polypeptide) by an aminoacyl (aaRS or RS)-tRNA synthetase-tRNA pair. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNA_{CUA} pairs, E. coli LeuRS (Ec-Leu)/B. stearothermophilus tRNA_{CUA} pairs, and pyrrolysyl-tRNA pairs. In some instances, the unnatural amino acid is incorporated into the cytokine (e.g., the IL polypeptide) by a Mj-TyrRS/tRNA pair. Exemplary UAAs that can be incorporated by a Mj-TyrRS/tRNA pair include, but are not limited to, para-substituted phenylalanine derivatives such as p-aminophenylalanine and p-methoyphenylalanine; meta-substituted tyrosine derivatives such as 3-aminotyrosine, 3-nitrotyrosine, 3,4-dihydroxyphenylalanine, and 3-iodotyrosine; phenyl selenocysteine; p-boronophenylalanine; and o-nitrobenzyltyrosine.

In some instances, the unnatural amino acid is incorporated into the cytokine (e.g., the IL polypeptide) by a Ec-Tyr/tRNA_{CUA} or a Ec-Leu/tRNA_{CUA} pair. Exemplary UAAs that can be incorporated by a Ec-Tyr/tRNA_{CUA} or a Ec-Leu/tRNA_{CUA} pair include, but are not limited to, phenylalanine derivatives containing benzophenone, ketone, iodide, or azide substituents; O-propargyltyrosine; α-aminocaprylic acid, O-methyl tyrosine, O-nitrobenzyl cysteine; and 3-(naphthalene-2-ylamino)-2-amino-propanoic acid.

In some instances, the unnatural amino acid is incorporated into the cytokine (e.g., the IL polypeptide) by a pyrrolysyl-tRNA pair. In some cases, the PylRS is obtained from an archaebacterial, e.g., from a methanogenic archaebacterial. In some cases, the PylRS is obtained from Methanosarcina barkeri, Methanosarcina mazei, or Methanosarcina acetivorans. Exemplary UAAs that can be incorporated by a pyrrolysyl-tRNA pair include, but are not limited to, amide and carbamate substituted lysines such as 2-amino-6-((R)-tetrahydrofuran-2-carboxamido)hexanoic acid, N-ε-D-prolyl-L-lysine, and N-ε-cyclopentyloxycarbonyl-L-lysine; N-ε-Acryloyl-L-lysine; N-ε-[(1-(6-nitrobenzo[d][1,3]dioxol-5-yl)ethoxy)carbonyl]-L-lysine; and N-ε-(1-methylcyclopro-2-enecarboxamido)lysine. In some embodiments, the IL-2 conjugates disclosed herein may be prepared by use of M. mazei tRNA which is selectively charged with a non-natural amino acid such as N6-((2-azidoethoxy)-carbonyl)-L-lysine (AzK) by the M. barkeri pyrrolysyl-tRNA synthetase (Mb PylRS). Other methods are known to those of ordinary skill in the art, such as those disclosed in Zhang et al., Nature 2017, 551(7682): 644-647, the disclosure of which is incorporated herein by reference.

In some instances, an unnatural amino acid is incorporated into a cytokine described herein (e.g., the IL polypeptide) by a synthetase disclosed in U.S. Pat. Nos. 9,988,619 and 9,938,516, the disclosure of each of which is incorporated herein by reference.

The host cell into which the constructs or vectors disclosed herein are introduced is cultured or maintained in a suitable medium such that the tRNA, the tRNA synthetase and the protein of interest are produced. The medium also comprises the unnatural amino acid(s) such that the protein of interest incorporates the unnatural amino acid(s). In some embodiments, a nucleoside triphosphate transporter (NTT) from bacteria, plant, or algae is also present in the host cell. In some embodiments, the IL-2 conjugates disclosed herein are prepared by use of a host cell that expresses a NTT. In some embodiments, the nucleotide nucleoside triphosphate transporter used in the host cell may be selected from TpNTT1, TpNTT2, TpNTT3, TpNTT4, TpNTT5, TpNTT6, TpNTT7, TpNTT8 (T. pseudonana), PtNTT1, PtNTT2, PtNTT3, PtNTT4, PtNTT5, PtNTT6 (P. tricornutum), GsNTT (Galdieria sulphuraria), AtNTT1, AtNTT2 (Arabidopsis thaliana), CtNTT1, CtNTT2 (Chlamydia trachomatis), PamNTT1, PamNTT2 (Protochlamydia amoebophila), CcNTT (Caedibacter caryophilus), RpNTT1 (Rickettsia prowazekii). In some embodiments, the NTT is selected from PtNTT1, PtNTT2, PtNTT3, PtNTT4, PtNTT5, and PtNTT6. In some embodiments, the NTT is PtNTT1. In some embodiments, the NTT is PtNTT2. In some embodiments, the NTT is PtNTT3. In some embodiments, the NTT is PtNTT4. In some embodiments, the NTT is PtNTT5. In some embodiments, the NTT is PtNTT6. Other NTTs that may be used are disclosed in Zhang et al., Nature 2017, 551(7682): 644-647; Malyshev et al. Nature 2014 (509(7500), 385-388; and Zhang et al. Proc Natl Acad Sci USA, 2017, 114:1317-1322.

The orthogonal tRNA synthetase/tRNA pair charges a tRNA with an unnatural amino acid and incorporates the unnatural amino acid into the polypeptide chain in response to the codon. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNA_{CUA} pairs, E. coli LeuRS (Ec-Leu)/B. stearothermophilus tRNA_{CUA} pairs, and pyrrolysyl-tRNA pairs. Other aaRS-tRNA pairs that may be used according to the present disclosure include those derived from M. mazei those described in Feldman et al., J Am Chem Soc., 2018 140:1447-1454; and Zhang et al. Proc Natl Acad Sci USA, 2017, 114:1317-1322; the disclosure of each of which is incorporated herein by reference.

In some embodiments are provided methods of preparing the IL-2 conjugates disclosed herein in a cellular system that expresses a NTT and a tRNA synthetase. In some embodiments described herein, the NTT is selected from PtNTT1, PtNTT2, PtNTT3, PtNTT4, PtNTT5, and PtNTT6, and the tRNA synthetase is selected from Methanococcus jannaschii, E. coli TyrRS (Ec-Tyr)/B. stearothermophilus, and M. mazei. In some embodiments, the NTT is PtNTT1 and the tRNA synthetase is derived from Methanococcus jannaschii, E. coli TyrRS (Ec-Tyr)/B. stearothermophilus, or M. mazei. In some embodiments, the NTT is PtNTT2 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT4 and the tRNA synthetase is derived from Methanococcus jannaschii, E. coli TyrRS (Ec-Tyr)/B. stearothermophilus, or M. mazei. In some embodiments, the NTT is PtNTT5 and the tRNA synthetase is derived from Methanococcus jannaschii, E. coli TyrRS (Ec-Tyr)/B. stearothermophilus, or M. mazei. In some embodiments, the NTT is PtNTT6 and the tRNA synthetase is derived from Methanococcus jannaschii, E. coli TyrRS (Ec-Tyr)/B. stearothermophilus, or M. mazei.

In some embodiments, the IL-2 conjugates disclosed herein may be prepared in a cell, such as E. coli, comprising (a) nucleotide triphosphate transporter PtNTT2 (including a truncated variant in which the first 65 amino acid residues of the full-length protein are deleted), (b) a plasmid comprising a double-stranded oligonucleotide that encodes an IL-2 variant having a desired amino acid sequence and that contains a unnatural base pair comprising a first unnatural nucleotide and a second unnatural nucleotide to provide a codon at the desired position at which an unnatural amino acid, such as N6-((2-azidoethoxy)-carbonyl)-L-lysine (AzK), will be incorporated, (c) a plasmid encoding a tRNA derived from M. mazei and which comprises an unnatural nucleotide to provide a recognized anticodon (to the codon of the IL-2 variant) in place of its native sequence, and (d) a plasmid encoding a M. barkeri derived pyrrolysyl-tRNA synthetase (Mb PylRS), which may be the same plasmid that encodes the tRNA or a different plasmid. In some embodiments, the cell is further supplemented with deoxyribo triphosphates comprising one or more unnatural bases. In some embodiments, the cell is further supplemented with ribo triphosphates comprising one or more unnatural bases. In some embodiments, the cells is further supplemented with one or more unnatural amino acids, such as N6-((2-azidoethoxy)-carbonyl)-L-lysine (AzK). In some embodiments, the double-stranded oligonucleotide that encodes the amino acid sequence of the desired IL-2 variant contains a codon AXC at position 64 of the sequence that encodes the protein having SEQ ID NO: 1, wherein X is an unnatural nucleotide. In some embodiments, the cell further comprises a plasmid, which may be the protein expression plasmid or another plasmid, that encodes an orthogonal tRNA gene from M. mazei that comprises an AXC-matching anticodon GYT in place of its native sequence, wherein Y is an unnatural nucleotide that is complementary and may be the same or different as the unnatural nucleotide in the codon. In some embodiments, the unnatural nucleotide in the codon is different than and complimentary to the unnatural nucleotide in the anti-codon. In some embodiments, the unnatural nucleotide in the codon is the same as the unnatural nucleotide in the anti-codon. In some embodiments, the first and second unnatural nucleotides comprising the unnatural base pair in the double-stranded oligonucleotide may be derived from

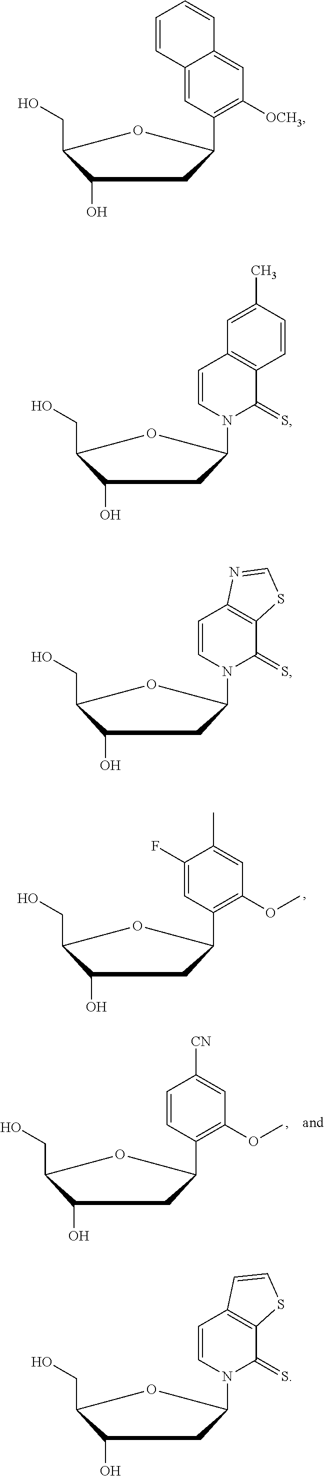

In some embodiments, the first and second unnatural nucleotides comprising the unnatural base pair in the double-stranded oligonucleotide may be derived from

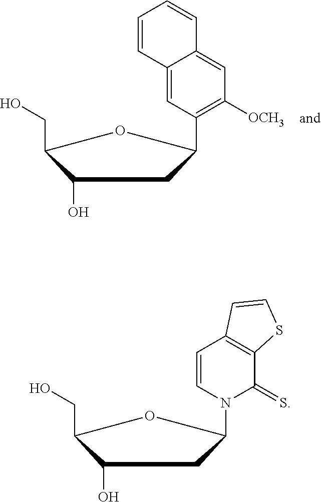

In some embodiments, the triphosphates of the first and second unnatural nucleotides include,

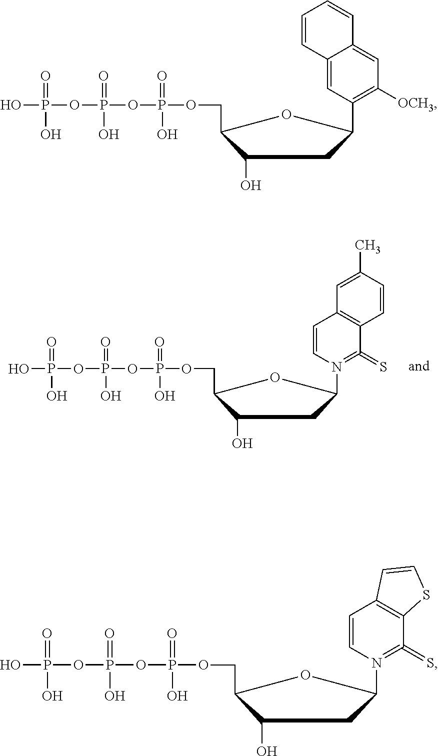

or salts thereof. In some embodiments, the triphosphates of the first and second unnatural nucleotides include,

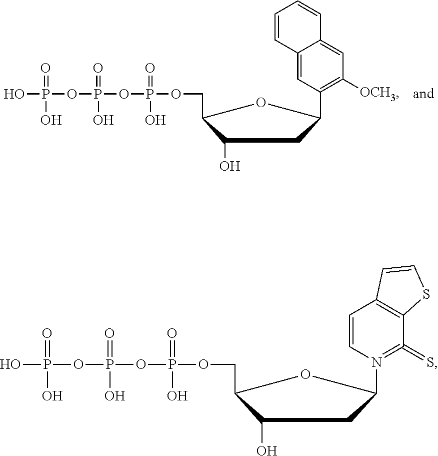

or salts thereof. In some embodiments, the mRNA derived the double-stranded oligonucleotide comprising a first unnatural nucleotide and a second unnatural nucleotide may comprise a codon comprising an unnatural nucleotide derived from

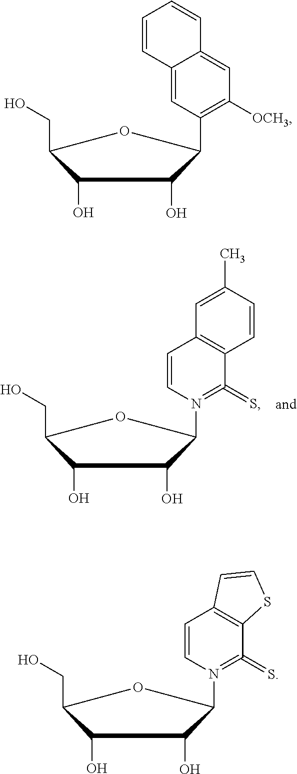

In some embodiments, the M. mazei tRNA may comprise an anti-codon comprising an unnatural nucleotide that recognizes the codon comprising the unnatural nucleotide of the mRNA. The anti-codon in the M. mazei tRNA may comprise an unnatural nucleotide derived from

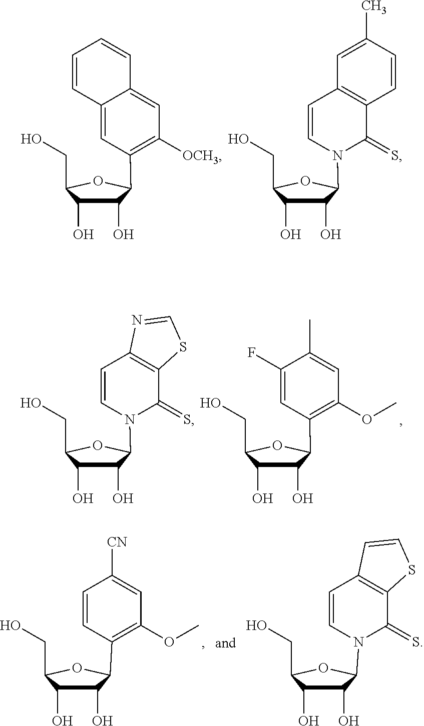

In some embodiments, the mRNA comprises an unnatural nucleotide derived from

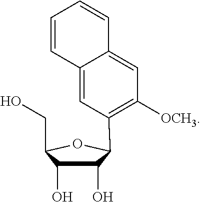

In some embodiments, the mRNA comprises an unnatural nucleotide derived from

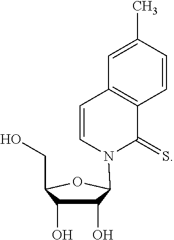

In some embodiments, the mRNA comprises an unnatural nucleotide derived from

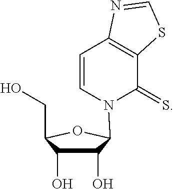

In some embodiments, the mRNA comprises an unnatural nucleotide derived from

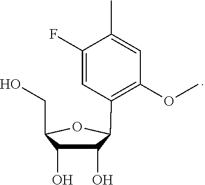

In some embodiments, the mRNA comprises an unnatural nucleotide derived from

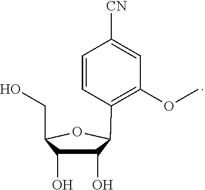

In some embodiments, the mRNA comprises an unnatural nucleotide derived from

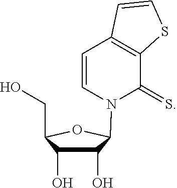

In some embodiments, the tRNA comprises an unnatural nucleotide derived from

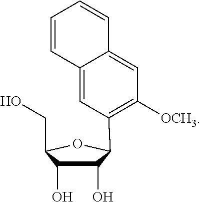

In some embodiments, the tRNA comprises an unnatural nucleotide derived from

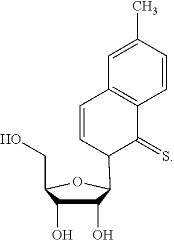

In some embodiments, the tRNA comprises an unnatural nucleotide derived from

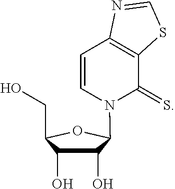

In some embodiments, the tRNA comprises an unnatural nucleotide derived from

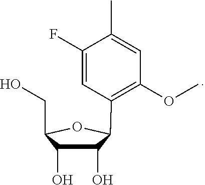

In some embodiments, the tRNA comprises an unnatural nucleotide derived from

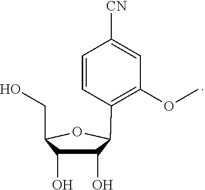

In some embodiments, the tRNA comprises an unnatural nucleotide derived from

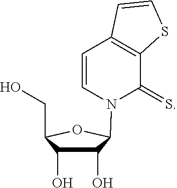

In some embodiments, the mRNA comprises an unnatural nucleotide derived from

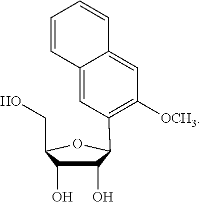

and the tRNA comprises an unnatural nucleotide derived from

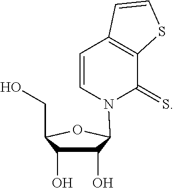

In some embodiments, the mRNA comprises an unnatural nucleotide derived from

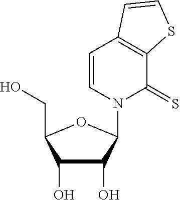

and the tRNA comprises an unnatural nucleotide derived from

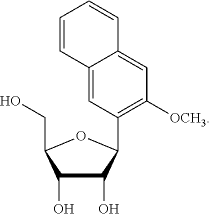

In some embodiments, the mRNA comprises an unnatural nucleotide derived from

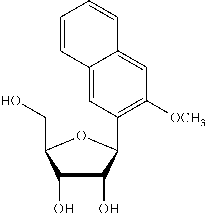

and the tRNA comprises an unnatural nucleotide derived from

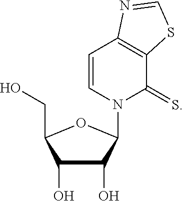

In some embodiments, the mRNA comprises an unnatural nucleotide derived from

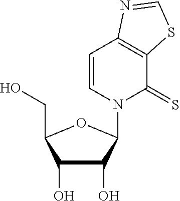

and the tRNA comprises an unnatural nucleotide derived from

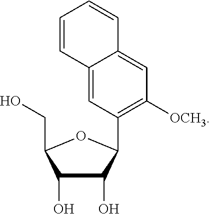

The host cell is cultured in a medium containing appropriate nutrients, and is supplemented with (a) the triphosphates of the deoxyribo nucleosides comprising one or more unnatural bases that are necessary for replication of the plasmid(s) encoding the cytokine gene harboring the codon, (b) the triphosphates of the ribo nucleosides comprising one or more unnatural bases necessary for transcription of (i) the mRNA corresponding to the coding sequence of the cytokine and containing the codon comprising one or more unnatural bases, and (ii) the tRNA containing the anticodon comprising one or more unnatural bases, and (c) the unnatural amino acid(s) to be incorporated in to the polypeptide sequence of the cytokine of interest. The host cells are then maintained under conditions which permit expression of the protein of interest.

The resulting AzK-containing protein that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551(7682): 644-647; WO 2015157555; WO 2015021432; WO 2016115168; WO 2017106767; WO 2017223528; WO 2019014262; WO 2019014267; WO 2019028419; and WO2019/028425; the disclosure of each of which is incorporated herein by reference.

The resulting protein comprising the one or more unnatural amino acids, Azk for example, that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551(7682): 644-647; WO 2015157555; WO 2015021432; WO 2016115168; WO 2017106767; WO 2017223528; WO 2019014262; WO 2019014267; WO 2019028419; and WO2019/028425; the disclosure of each of which is incorporated herein by reference.

Alternatively, an IL-2 polypeptide comprising an unnatural amino acid(s) is prepared by introducing the nucleic acid constructs described herein comprising the tRNA and aminoacyl tRNA synthetase and comprising a nucleic acid sequence of interest with one or more in-frame orthogonal (stop) codons into a host cell. The host cell is cultured in a medium containing appropriate nutrients, is supplemented with (a) the triphosphates of the deoxyribo nucleosides comprising one or more unnatural bases required for replication of the plasmid(s) encoding the cytokine gene harboring the new codon and anticodon, (b) the triphosphates of the ribo nucleosides required for transcription of the mRNA corresponding to (i) the cytokine sequence containing the codon, and (ii) the orthogonal tRNA containing the anticodon, and (c) the unnatural amino acid(s). The host cells are then maintained under conditions which permit expression of the protein of interest. The unnatural amino acid(s) is incorporated into the polypeptide chain in response to the unnatural codon. For example, one or more unnatural amino acids are incorporated into the IL-2 polypeptide. Alternatively, two or more unnatural amino acids may be incorporated into the IL-2 polypeptide at two or more sites in the protein.

Once the IL-2 polypeptide incorporating the unnatural amino acid(s) has been produced in the host cell it can be extracted therefrom by a variety of techniques known in the art, including enzymatic, chemical and/or osmotic lysis and physical disruption. The IL-2 polypeptide can be purified by standard techniques known in the art such as preparative ion exchange chromatography, hydrophobic chromatography, affinity chromatography, or any other suitable technique known to those of ordinary skill in the art.

Suitable host cells may include bacterial cells (e.g., E. coli, BL21(DE3)), but most suitably host cells are eukaryotic cells, for example insect cells (e.g. Drosophila such as Drosophila melanogaster), yeast cells, nematodes (e.g. C. elegans), mice (e.g. Mus musculus), or mammalian cells (such as Chinese hamster ovary cells (CHO) or COS cells, human 293T cells, HeLa cells, NIH 3T3 cells, and mouse erythroleukemia (MEL) cells) or human cells or other eukaryotic cells. Other suitable host cells are known to those skilled in the art. Suitably, the host cell is a mammalian cell—such as a human cell or an insect cell. In some embodiments, the suitable host cells comprise E. coli.

Other suitable host cells which may be used generally in the embodiments of the invention are those mentioned in the examples section. Vector DNA can be introduced into host cells via conventional transformation or transfection techniques. As used herein, the terms “transformation” and “transfection” are intended to refer to a variety of well-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells are well known in the art.

When creating cell lines, it is generally preferred that stable cell lines are prepared. For stable transfection of mammalian cells for example, it is known that, depending upon the expression vector and transfection technique used, only a small fraction of cells may integrate the foreign DNA into their genome. In order to identify and select these integrants, a gene that encodes a selectable marker (for example, for resistance to antibiotics) is generally introduced into the host cells along with the gene of interest. Preferred selectable markers include those that confer resistance to drugs, such as G418, hygromycin, or methotrexate. Nucleic acid molecules encoding a selectable marker can be introduced into a host cell on the same vector or can be introduced on a separate vector. Cells stably transfected with the introduced nucleic acid molecule can be identified by drug selection (for example, cells that have incorporated the selectable marker gene will survive, while the other cells die).

In one embodiment, the constructs described herein are integrated into the genome of the host cell. An advantage of stable integration is that the uniformity between individual cells or clones is achieved. Another advantage is that selection of the best producers may be carried out. Accordingly, it is desirable to create stable cell lines. In another embodiment, the constructs described herein are transfected into a host cell. An advantage of transfecting the constructs into the host cell is that protein yields may be maximized. In one aspect, there is described a cell comprising the nucleic acid construct or the vector described herein.

### Methods of Treatment

In one aspect, provided herein is a method of treating a cancer in a subject in need thereof, comprising administering to the subject (a) about 8 μg/kg, 16 μg/kg, 24 μg/kg, or 32 μg/kg of an IL-2 conjugate as described herein, and (b) pembrolizumab. In some embodiments, the method of treating a cancer in a subject in need thereof comprises administering to the subject (a) about 8 μg/kg of an IL-2 conjugate as described herein, and (b) pembrolizumab. In some embodiments, the method of treating a cancer in a subject in need thereof comprises administering to the subject (a) about 16 μg/kg of an IL-2 conjugate as described herein, and (b) pembrolizumab. In some embodiments, the method of treating a cancer in a subject in need thereof comprises administering to the subject (a) about 24 μg/kg of an IL-2 conjugate as described herein, and (b) pembrolizumab. In some embodiments, the method of treating a cancer in a subject in need thereof comprises administering to the subject (a) about 32 μg/kg of an IL-2 conjugate as described herein, and (b) pembrolizumab.

In another aspect, provided herein is an IL-2 conjugate for use in a method of treating a cancer in a subject in need thereof, the method comprising administering to the subject (a) about 8 μg/kg, 16 μg/kg, 24 μg/kg, or 32 μg/kg of an IL-2 conjugate as described herein, and (b) pembrolizumab. In some embodiments, the method comprises administering to the subject (a) about 8 μg/kg of an IL-2 conjugate as described herein, and (b) pembrolizumab. In some embodiments, the method comprises administering to the subject (a) about 16 μg/kg of an IL-2 conjugate as described herein, and (b) pembrolizumab. In some embodiments, the method comprises administering to the subject (a) about 24 μg/kg of an IL-2 conjugate as described herein, and (b) pembrolizumab. In some embodiments, the method comprises administering to the subject (a) about 32 μg/kg of an IL-2 conjugate as described herein, and (b) pembrolizumab.

In a further aspect, provided herein is use of an IL-2 conjugate for the manufacture of a medicament for a method of treating a cancer in a subject in need thereof, the method comprising administering to the subject (a) about 8 μg/kg, 16 μg/kg, 24 μg/kg, or 32 μg/kg of an IL-2 conjugate as described herein, and (b) pembrolizumab. In some embodiments, the method comprises administering to the subject (a) about 8 μg/kg of an IL-2 conjugate as described herein, and (b) pembrolizumab. In some embodiments, the method comprises administering to the subject (a) about 16 μg/kg of an IL-2 conjugate as described herein, and (b) pembrolizumab. In some embodiments, the method comprises administering to the subject (a) about 24 μg/kg of an IL-2 conjugate as described herein, and (b) pembrolizumab. In some embodiments, the method comprises administering to the subject (a) about 32 μg/kg of an IL-2 conjugate as described herein, and (b) pembrolizumab.

In some aspects, provided herein is use of an IL-2 conjugate for stimulating CD8+ and/or NK cells in a subject in need thereof, the method comprising administering to the subject (a) about 8 μg/kg, 16 μg/kg, 24 μg/kg, or 32 μg/kg of an IL-2 conjugate as described herein, and (b) pembrolizumab. In some embodiments, the method comprises administering to the subject (a) about 8 μg/kg of an IL-2 conjugate as described herein, and (b) pembrolizumab. In some embodiments, the method comprises administering to the subject (a) about 16 μg/kg of an IL-2 conjugate as described herein, and (b) pembrolizumab. In some embodiments, the method comprises administering to the subject (a) about 24 μg/kg of an IL-2 conjugate as described herein, and (b) pembrolizumab. In some embodiments, the method comprises administering to the subject (a) about 32 μg/kg of an IL-2 conjugate as described herein, and (b) pembrolizumab.

### Cancer Types

In some embodiments, the cancer is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, gastric cancer, colon cancer, colorectal cancer (CRC), cervical cancer, hepatocellular carcinoma (HCC), Merkel cell carcinoma (MCC), melanoma, small cell lung cancer (SCLC), esophageal, esophageal squamous cell carcinoma (ESCC), glioblastoma, mesothelioma, breast cancer, triple-negative breast cancer, prostate cancer, castrate-resistant prostate cancer, metastatic castrate-resistant prostate cancer, or metastatic castrate-resistant prostate cancer having DNA damage response (DDR) defects, bladder cancer, ovarian cancer, tumors of moderate to low mutational burden, cutaneous squamous cell carcinoma (CSCC), squamous cell skin cancer (SCSC), tumors of low- to non-expressing PD-L1, tumors disseminated systemically to the liver and CNS beyond their primary anatomic originating site, and diffuse large B-cell lymphoma (DLBCL).

In some embodiments, the cancer in the subject is selected from renal cell carcinoma (RCC), non-small cell lung cancer (NSCLC), urothelial carcinoma, melanoma, Merkel cell carcinoma (MCC), and head and neck squamous cell cancer (HNSCC). In some embodiments, the cancer is renal cell carcinoma (RCC). In some embodiments, the cancer is non-small cell lung cancer (NSCLC). In some embodiments, the cancer is urothelial carcinoma. In some embodiments, the cancer is melanoma. In some embodiments, the cancer is Merkel cell carcinoma (MCC). In some embodiments, the cancer is head and neck squamous cell cancer (HNSCC).

In some embodiments, the cancer is in the form of a solid tumor. In some embodiments, the cancer is an advanced or metastatic solid tumor. In some embodiments, the cancer is in the form of a liquid tumor. In some embodiments, the cancer is refractory cancer. In some embodiments, the cancer is relapsed cancer.

### Administration

In some embodiments, the IL-2 conjugate is administered to the subject by intravenous, subcutaneous, intramuscular, intracerebral, intranasal, intra-arterial, intra-articular, intradermal, intravitreal, intraosseous infusion, intraperitoneal, or intrathecal administration. In some embodiments, the IL-2 conjugate is administered to the subject by intravenous, subcutaneous, or intramuscular administration. In some embodiments, the IL-2 conjugate is administered to the subject by subcutaneous or intravenous administration. In some embodiments, the IL-2 conjugate is administered to the subject by intravenous administration. In some embodiments, the IL-2 conjugate is administered to the subject by subcutaneous administration. In some embodiments, the IL-2 conjugate is administered to the subject by intramuscular administration. In some embodiments, the IL-2 conjugate and pembrolizumab are administered to the subject by intravenous administration.

The IL-2 conjugate may be administered more than once, e.g., twice, three times, four times, five times, or more. In some embodiments, the duration of the treatment is up to 24 months, such as 1 month, 2 months, 3 months, 6 months, 9 months, 12 months, 15 months, 18 months, 21 months or 24 months. In some embodiments, the duration of treatment is further extended by up to another 24 months.

In some embodiments, the IL-2 conjugate is administered to the subject separately from the administration of pembrolizumab. In some embodiments, the IL-2 conjugate and pembrolizumab are administered to the subject sequentially. In some embodiments, the IL-2 conjugate is administered to the subject prior to the administration to the subject of pembrolizumab. In some embodiments, the IL-2 conjugate is administered to the subject after the administration to the subject of pembrolizumab. In some embodiments, the IL-2 conjugate and pembrolizumab are administered to the subject simultaneously.

In some embodiments, the IL-2 conjugate is administered to a subject in need thereof about once every two weeks, about once every three weeks, or about once every 4 weeks. In some embodiments, the IL-2 conjugate is administered to a subject in need thereof once every two weeks. In some embodiments, the IL-2 conjugate is administered to a subject in need thereof once every three weeks. In some embodiments, the IL-2 conjugate is administered to a subject in need thereof once every 4 weeks. In some embodiments, the IL-2 conjugate is administered about once every 14, 15, 16, 17, 18, 19, 20, or 21 days.

In some embodiments, pembrolizumab is administered to a subject in need thereof about once every two weeks, about once every three weeks, or about once every 4 weeks. In some embodiments, pembrolizumab is administered to a subject in need thereof once every two weeks. In some embodiments, pembrolizumab is administered to a subject in need thereof once every three weeks. In some embodiments, pembrolizumab is administered to a subject in need thereof once every 4 weeks. In some embodiments, pembrolizumab is administered about once every 14, 15, 16, 17, 18, 19, 20, or 21 days.

In some embodiments, the IL-2 conjugate and pembrolizumab are administered to a subject in need thereof about once every two weeks, about once every three weeks, or about once every 4 weeks. In some embodiments, the IL-2 conjugate and pembrolizumab are administered to a subject in need thereof once every two weeks. In some embodiments, the IL-2 conjugate and pembrolizumab are administered to a subject in need thereof once every three weeks. In some embodiments, the IL-2 conjugate and pembrolizumab are administered to a subject in need thereof once every 4 weeks. In some embodiments, the IL-2 conjugate and pembrolizumab are administered about once every 14, 15, 16, 17, 18, 19, 20, or 21 days.

In some instances, the desired doses are conveniently presented in a single dose or as divided doses administered simultaneously (or over a short period of time) or at appropriate intervals, for example as two, three, four or more sub-doses per day.

In some embodiments, pembrolizumab is administered at a dose of about 200 mg every 3 weeks.

### Subject

In some embodiments, administration of the IL-2 conjugate and pembrolizumab is to an adult. In some embodiments, the adult is a male. In other embodiments, the adult is a female. In some embodiments, the adult is at least age 20, 25, 30, 35, 40, 45, 50, 55, 60, 65, 70, 75, 80, 85, 90, or 95 years of age. In some embodiments, administration of the IL-2 conjugate and pembrolizumab is to an infant, child, or adolescent. In some embodiments, the subject is at least 1 month, 2 months, 3 months, 6 months, 9 months or 12 months of age. In some embodiments, the subject is at least 1, 2, 3, 4, 5, 6, 7, 8, 9, 10, 11, 12, 13, 14, 15, 16, 17, 18, or 19 years of age.

In some embodiments, the subject has measurable disease (i.e., cancer) as determined by RECIST v1.1. In some embodiments, the subject has been determined to have Eastern Cooperative Oncology Group (ECOG) performance status of 0 or 1. In some embodiments, the subject has adequate cardiovascular, hematological, liver, and renal function, as determined by a physician. In some embodiments, the subject has been determined (e.g., by a physician) to have a life expectancy greater than or equal to 12 weeks. In some embodiments, the subject has had prior anti-cancer therapy before administration of the first treatment dose.

In some embodiments, the subject has a solid tumor cancer. In some embodiments, the subject has a metastatic solid tumor. In some embodiments, the subject has an advanced solid tumor. In some embodiments, the subject has refractory cancer. In some embodiments, the subject has relapsed cancer.

In some embodiments, the subject has no known hypersensitivity or contraindications to any of the IL-2 conjugates disclosed herein, PEG, pegylated drugs, or pembrolizumab.

### Effects of Administration

In some embodiments, administration of the IL-2 conjugate and pembrolizumab provides a complete response, a partial response or stable disease.

In some embodiments, following administration of the IL-2 conjugate and pembrolizumab, the subject experiences a response as measured by the Immune-related Response Evaluation Criteria in Solid Tumors (iRECIST). In some embodiments, following administration of the IL-2 conjugate and pembrolizumab, the subject experiences an Objective Response Rate (ORR) according to RECIST version 1.1. In some embodiments, following administration of the IL-2 conjugate and pembrolizumab, the subject experiences Duration of Response (DOR) according to RECIST versions 1.1. In some embodiments, following administration of the IL-2 conjugate and pembrolizumab, the subject experiences Progression-Free Survival (PFS) according to RECIST version 1.1. In some embodiments, following administration of the IL-2 conjugate and pembrolizumab, the subject experiences Overall Survival according to RECIST version 1.1. In some embodiments, following administration of the IL-2 conjugate and pembrolizumab, the subject experiences Time to Response (TTR) according to RECIST version 1.1. In some embodiments, following administration of the IL-2 conjugate and pembrolizumab, the subject experiences Disease Control Rate (DCR) according to RECIST version 1.1. In any of these embodiments, the subject's experience is based on a physician's review of a radiographic image taken of the subject.

In some embodiments, administration of the IL-2 conjugate and pembrolizumab to the subject does not cause vascular leak syndrome in the subject. In some embodiments, administration of the IL-2 conjugate and pembrolizumab to the subject does not cause Grade 2, Grade 3, or Grade 4 vascular leak syndrome in the subject. In some embodiments, administration of the IL-2 conjugate and pembrolizumab to the subject does not cause Grade 2 vascular leak syndrome in the subject. In some embodiments, administration of the IL-2 conjugate and pembrolizumab to the subject does not cause Grade 3 vascular leak syndrome in the subject. In some embodiments, administration of the IL-2 conjugate and pembrolizumab to the subject does not cause Grade 4 vascular leak syndrome in the subject. In some embodiments, administration of the IL-2 conjugate and pembrolizumab to the subject does not cause loss of vascular tone in the subject.

In some embodiments, administration of the IL-2 conjugate and pembrolizumab to the subject does not cause extravasation of plasma proteins and fluid into the extravascular space in the subject.

In some embodiments, administration of the IL-2 conjugate and pembrolizumab to the subject does not cause hypotension and reduced organ perfusion in the subject.

In some embodiments, administration of the IL-2 conjugate and pembrolizumab to the subject does not cause impaired neutrophil function in the subject. In some embodiments, administration of the IL-2 conjugate and pembrolizumab to the subject does not cause reduced chemotaxis in the subject.

In some embodiments, administration of the IL-2 conjugate and pembrolizumab to the subject is not associated with an increased risk of disseminated infection in the subject. In some embodiments, the disseminated infection is sepsis or bacterial endocarditis. In some embodiments, the disseminated infection is sepsis. In some embodiments, the disseminated infection is bacterial endocarditis. In some embodiments, the subject is treated for any preexisting bacterial infections prior to administration of the IL-2 conjugate and pembrolizumab. In some embodiments, the subject is treated with an antibacterial agent selected from oxacillin, nafcillin, ciprofloxacin, and vancomycin prior to administration of the IL-2 conjugate and pembrolizumab.

In some embodiments, administration of the IL-2 conjugate and pembrolizumab to the subject does not exacerbate a pre-existing or initial presentation of an autoimmune disease or an inflammatory disorder in the subject. In some embodiments, the administration of the IL-2 conjugate and pembrolizumab to the subject does not exacerbate a pre-existing or initial presentation of an autoimmune disease in the subject. In some embodiments, the administration of the IL-2 conjugate and pembrolizumab to the subject does not exacerbate a pre-existing or initial presentation of an inflammatory disorder in the subject. In some embodiments, the autoimmune disease or inflammatory disorder in the subject is selected from Crohn's disease, scleroderma, thyroiditis, inflammatory arthritis, diabetes mellitus, oculo-bulbar myasthenia gravis, crescentic IgA glomerulonephritis, cholecystitis, cerebral vasculitis, Stevens-Johnson syndrome and bullous pemphigoid. In some embodiments, the autoimmune disease or inflammatory disorder in the subject is Crohn's disease. In some embodiments, the autoimmune disease or inflammatory disorder in the subject is scleroderma. In some embodiments, the autoimmune disease or inflammatory disorder in the subject is thyroiditis. In some embodiments, the autoimmune disease or inflammatory disorder in the subject is inflammatory arthritis. In some embodiments, the autoimmune disease or inflammatory disorder in the subject is diabetes mellitus. In some embodiments, the autoimmune disease or inflammatory disorder in the subject is oculo-bulbar myasthenia gravis. In some embodiments, the autoimmune disease or inflammatory disorder in the subject is crescentic IgA glomerulonephritis. In some embodiments, the autoimmune disease or inflammatory disorder in the subject is cholecystitis. In some embodiments, the autoimmune disease or inflammatory disorder in the subject is cerebral vasculitis. In some embodiments, the autoimmune disease or inflammatory disorder in the subject is Stevens-Johnson syndrome. In some embodiments, the autoimmune disease or inflammatory disorder in the subject is bullous pemphigoid.

In some embodiments, administration of the IL-2 conjugate and pembrolizumab to the subject does not cause changes in mental status, speech difficulties, cortical blindness, limb or gait ataxia, hallucinations, agitation, obtundation, or coma in the subject. In some embodiments, administration of the IL-2 conjugate and pembrolizumab to the subject does not cause seizures in the subject. In some embodiments, administration of the IL-2 conjugate and pembrolizumab to the subject is not contraindicated in subjects having a known seizure disorder.

In some embodiments, administration of the IL-2 conjugate and pembrolizumab to the subject does not cause capillary leak syndrome in the subject. In some embodiments, administration of the IL-2 conjugate and pembrolizumab to the subject does not cause Grade 2, Grade 3, or Grade 4 capillary leak syndrome in the subject. In some embodiments, administration of the IL-2 conjugate and pembrolizumab to the subject does not cause Grade 2 capillary leak syndrome in the subject. In some embodiments, administration of the IL-2 conjugate and pembrolizumab to the subject does not cause Grade 3 capillary leak syndrome in the subject. In some embodiments, administration of the IL-2 conjugate and pembrolizumab to the subject does not cause Grade 4 capillary leak syndrome in the subject.

In some embodiments, administration of the IL-2 conjugate and pembrolizumab to the subject does not cause a drop in mean arterial blood pressure in the subject following administration. In some embodiments, administration of the IL-2 conjugate and pembrolizumab to the subject does cause hypotension in the subject. In some embodiments, administration of the IL-2 conjugate and pembrolizumab to the subject does not cause the subject to experience a systolic blood pressure below 90 mm Hg or a 20 mm Hg drop from baseline systolic pressure.

In some embodiments, administration of the IL-2 conjugate and pembrolizumab to the subject does not cause edema or impairment of kidney or liver function in the subject.

In some embodiments, administration of the IL-2 conjugate and pembrolizumab to the subject does not cause eosinophilia in the subject. In some embodiments, administration of the IL-2 conjugate and pembrolizumab to the subject does not cause the eosinophil count in the peripheral blood of the subject to exceed 500 per μL. In some embodiments, administration of the IL-2 conjugate and pembrolizumab to the subject does not cause the eosinophil count in the peripheral blood of the subject to exceed 500 μL to 1500 per μL. In some embodiments, administration of the IL-2 conjugate and pembrolizumab to the subject does not cause the eosinophil count in the peripheral blood of the subject to exceed 1500 per μL to 5000 per μL. In some embodiments, administration of the IL-2 conjugate and pembrolizumab to the subject does not cause the eosinophil count in the peripheral blood of the subject to exceed 5000 per μL. In some embodiments, administration of the IL-2 conjugate and pembrolizumab to the subject is not contraindicated in subjects on an existing regimen of psychotropic drugs.

In some embodiments, administration of the IL-2 conjugate and pembrolizumab to the subject is not contraindicated in subjects on an existing regimen of nephrotoxic, myelotoxic, cardiotoxic, or hepatotoxic drugs. In some embodiments, administration of the IL-2 conjugate and pembrolizumab to the subject is not contraindicated in subjects on an existing regimen of aminoglycosides, cytotoxic chemotherapy, doxorubicin, methotrexate, or asparaginase. In some embodiments, administration of the IL-2 conjugate and pembrolizumab to the subject is not contraindicated in subjects receiving combination regimens containing antineoplastic agents. In some embodiments, the antineoplastic agent is selected from dacarbazine, cis-platinum, tamoxifen and interferon-alpha.

In some embodiments, administration of the IL-2 conjugate and pembrolizumab to the subject does not cause one or more Grade 4 adverse events in the subject following administration. In some embodiments, Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis. In some embodiments, administration of the IL-2 conjugate and pembrolizumab to a group of subjects does not cause one or more Grade 4 adverse events in greater than 1% of the subjects following administration. In some embodiments, Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis.

In some embodiments, administration of the IL-2 conjugate and pembrolizumab to a group of subjects does not cause one or more adverse events in greater than 1% of the subjects following administration, wherein the one or more adverse events is selected from duodenal ulceration; bowel necrosis; myocarditis; supraventricular tachycardia; permanent or transient blindness secondary to optic neuritis; transient ischemic attacks; meningitis; cerebral edema; pericarditis; allergic interstitial nephritis; and tracheo-esophageal fistula.

In some embodiments, administration of the IL-2 conjugate and pembrolizumab to a group of subjects does not cause one or more adverse events in greater than 1% of the subjects following administration, wherein the one or more adverse events is selected from malignant hyperthermia; cardiac arrest; myocardial infarction; pulmonary emboli; stroke; intestinal perforation; liver or renal failure; severe depression leading to suicide; pulmonary edema; respiratory arrest; respiratory failure.

In some embodiments, administration of the IL-2 conjugate and pembrolizumab to the subject stimulates CD8+ cells in a subject. In some embodiments, administration of the IL-2 conjugate and pembrolizumab to the subject stimulates NK cells in a subject. Stimulation may comprise an increase in the number of CD8+ cells in the subject, e.g., about 4, 5, 6, or 7 days after administration, or about 1, 2, 3, or 4 weeks after administration. In some embodiments, the CD8+ cells comprise memory CD8+ cells. In some embodiments, the CD8+ cells comprise effector CD8+ cells. Stimulation may comprise an increase in the proportion of CD8+ cells that are Ki67 positive in the subject, e.g., about 4, 5, 6, or 7 days after administration, or about 1, 2, 3, or 4 weeks after administration. Stimulation may comprise an increase in the number of NK cells in the subject, e.g., about 4, 5, 6, or 7 days after administration, or about 1, 2, 3, or 4 weeks after administration.

In some embodiments, CD8+ cells are expanded in the subject following administration of the IL-2 conjugate and pembrolizumab by at least 1.5-fold, such as by at least 1.6-fold, 1.7-fold, 1.8-fold, or 1.9-fold. In some embodiments, NK cells are expanded in the subject following administration of the IL-2 conjugate and pembrolizumab by at least 5-fold, such as by at least 5.5-fold, 6-fold, or 6.5-fold. In some embodiments, eosinophils are expanded in the subject following administration of the IL-2 conjugate and pembrolizumab by no more than about 2-fold, such as no more than about 1.5-fold, 1.4-fold, or 1.3-fold. In some embodiments, CD4+ cells are expanded in the subject following administration of the IL-2 conjugate and pembrolizumab by no more than about 2-fold, such as no more than about 1.8-fold, 1.7-fold, or 1.6-fold. In some embodiments, the expansion of CD8+ cells and/or NK cells in the subject following administration of the IL-2 conjugate and pembrolizumab is greater than the expansion of CD4+ cells and/or eosinophils. In some embodiments, the expansion of CD8+ cells is greater than the expansion of CD4+ cells. In some embodiments, the expansion of NK cells is greater than the expansion of CD4+ cells. In some embodiments, the expansion of CD8+ cells is greater than the expansion of eosinophils. In some embodiments, the expansion of NK cells is greater than the expansion of eosinophils. Fold expansion is determined relative to a baseline value measured before administration of the IL-2 conjugate. In some embodiments, fold expansion is determined at any of the times after administration, such as about 4, 5, 6, or 7 days after administration, or about 1, 2, 3, or 4 weeks after administration.

In some embodiments, administration of the IL-2 conjugate and pembrolizumab to the subject increases the number of peripheral CD8+ T and NK cells in the subject without increasing the number of peripheral CD4+ regulatory T cells in the subject. In some embodiments, administration of the IL-2 conjugate and pembrolizumab to the subject increases the number of peripheral CD8+ T and NK cells in the subject without increasing the number of peripheral eosinophils in the subject. In some embodiments, administration of the IL-2 conjugate and pembrolizumab to the subject increases the number of peripheral CD8+ T and NK cells in the subject without increasing the number of intratumoral CD8+ T and NK cells in the subject and without increasing the number of intratumoral CD4+ regulatory T cells in the subject.

In some embodiments, administration of the IL-2 conjugate and pembrolizumab to the subject does not require the availability of an intensive care facility or skilled specialists in cardiopulmonary or intensive care medicine. In some embodiments, administration of the IL-2 conjugate and pembrolizumab to the subject does not require the availability of an intensive care facility or skilled specialists in cardiopulmonary or intensive care medicine. In some embodiments, administration of the IL-2 conjugate and pembrolizumab to the subject does not require the availability of an intensive care facility. In some embodiments, administration of the IL-2 conjugate and pembrolizumab to the subject does not require the availability of skilled specialists in cardiopulmonary or intensive care medicine.

In some embodiments, administration of the IL-2 conjugate and pembrolizumab does not cause dose-limiting toxicity. In some embodiments, administration of the IL-2 conjugate and pembrolizumab does not cause severe cytokine release syndrome. In some embodiments, the IL-2 conjugate does not induce anti-drug antibodies (ADAs), i.e., antibodies against the IL-2 conjugate. In some embodiments, a lack of induction of ADAs is determined by direct immunoassay for antibodies against PEG and/or ELISA for antibodies against the IL-2 conjugate. An IL-2 conjugate is considered not to induce ADAs if a measured level of ADAs is statistically indistinguishable from a baseline (pre-treatment) level or from a level in an untreated control.

### Additional Agents

In some embodiments, the methods further comprise administering to the subject a therapeutically effective amount of one or more chemotherapeutic agents, in addition to pembrolizumab. In some embodiments, the one or more chemotherapeutic agents comprises one or more platinum-based chemotherapeutic agents. In some embodiments, the one or more chemotherapeutic agents comprises carboplatin and pemetrexed. In some embodiments, the one or more chemotherapeutic agents comprises carboplatin and nab-paclitaxel. In some embodiments, the one or more chemotherapeutic agents comprises carboplatin and docetaxel. In some embodiments, the cancer in the subject is non-small cell lung cancer (NSCLC).

### Kits/Article of Manufacture

Disclosed herein, in certain embodiments, are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

### EXAMPLES

These examples are provided for illustrative purposes only and not to limit the scope of the claims provided herein.

### Example 1. Preparation of Pegylated IL-2 Conjugates

An exemplary method with details for preparing IL-2 conjugates described herein is provided in this Example.

IL-2 employed for bioconjugation was expressed as inclusion bodies in E. coli using methods disclosed herein, using: (a) an expression plasmid encoding (i) the protein with the desired amino acid sequence, which gene contains a first unnatural base pair to provide a codon at the desired position at which an unnatural amino acid N6-((2-azidoethoxy)-carbonyl)-L-lysine (AzK) was incorporated and (ii) a tRNA derived from M. mazei Pyl, which gene comprises a second unnatural nucleotide to provide a matching anticodon in place of its native sequence; (b) a plasmid encoding a M. barkeri derived pyrrolysyl-tRNA synthetase (Mb PylRS), (c) N6-((2-azidoethoxy)-carbonyl)-L-lysine (AzK); and (d) a truncated variant of nucleotide triphosphate transporter PtNTT2 in which the first 65 amino acid residues of the full-length protein were deleted. The double-stranded oligonucleotide that encodes the amino acid sequence of the desired IL-2 variant contained a codon AXC as codon 64 of the sequence that encodes the protein having SEQ ID NO: 1 in which P64 is replaced with an unnatural amino acid described herein. The plasmid encoding an orthogonal tRNA gene from M. mazei comprised an AXC-matching anticodon GYT in place of its native sequence, wherein Y is an unnatural nucleotide as disclosed herein. X and Y were selected from unnatural nucleotides dTPT3 and dNaM as disclosed herein. The expressed protein was extracted from inclusion bodies and re-folded using standard procedures before site-specifically pegylating the AzK-containing IL-2 product using DBCO-mediated copper-free click chemistry to attach stable, covalent mPEG moieties to the AzK. Exemplary reactions are shown in Schemes 1 and 2 (wherein n indicates the number of repeating PEG units). The reaction of the AzK moiety with the DBCO alkynyl moiety may afford one regioisomeric product or a mixture of regioisomeric products.

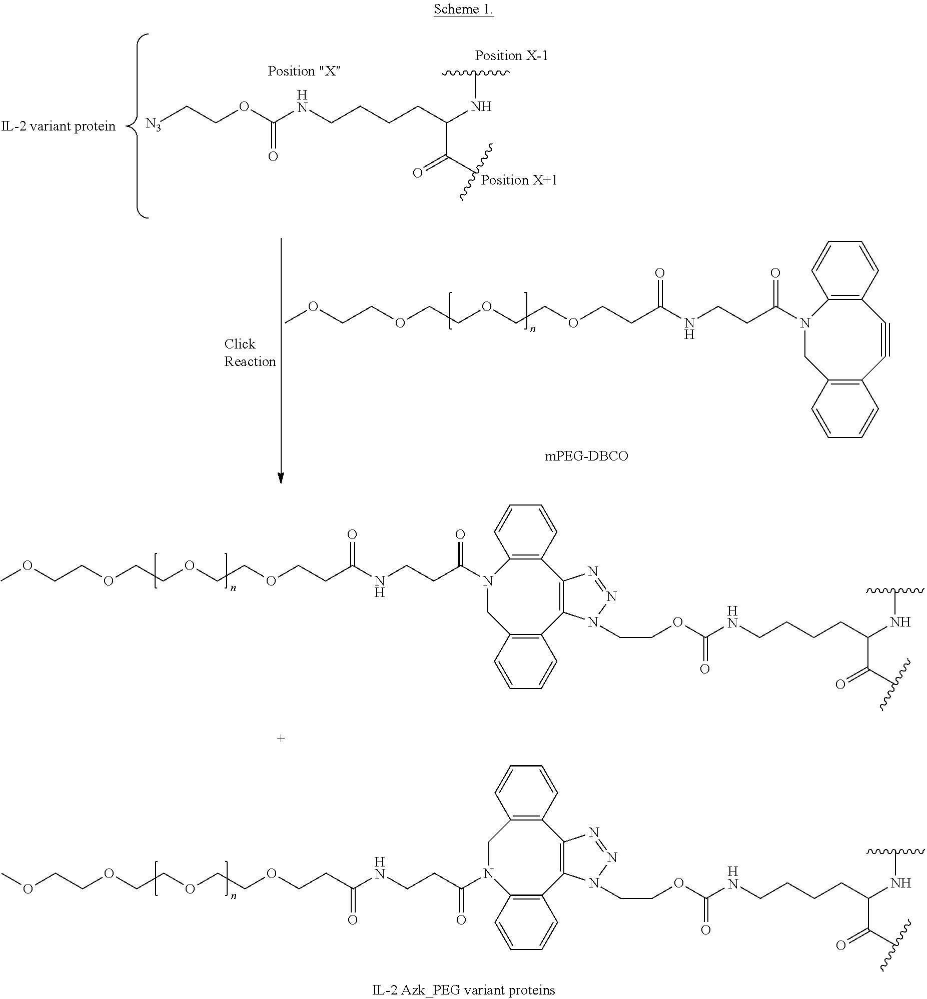

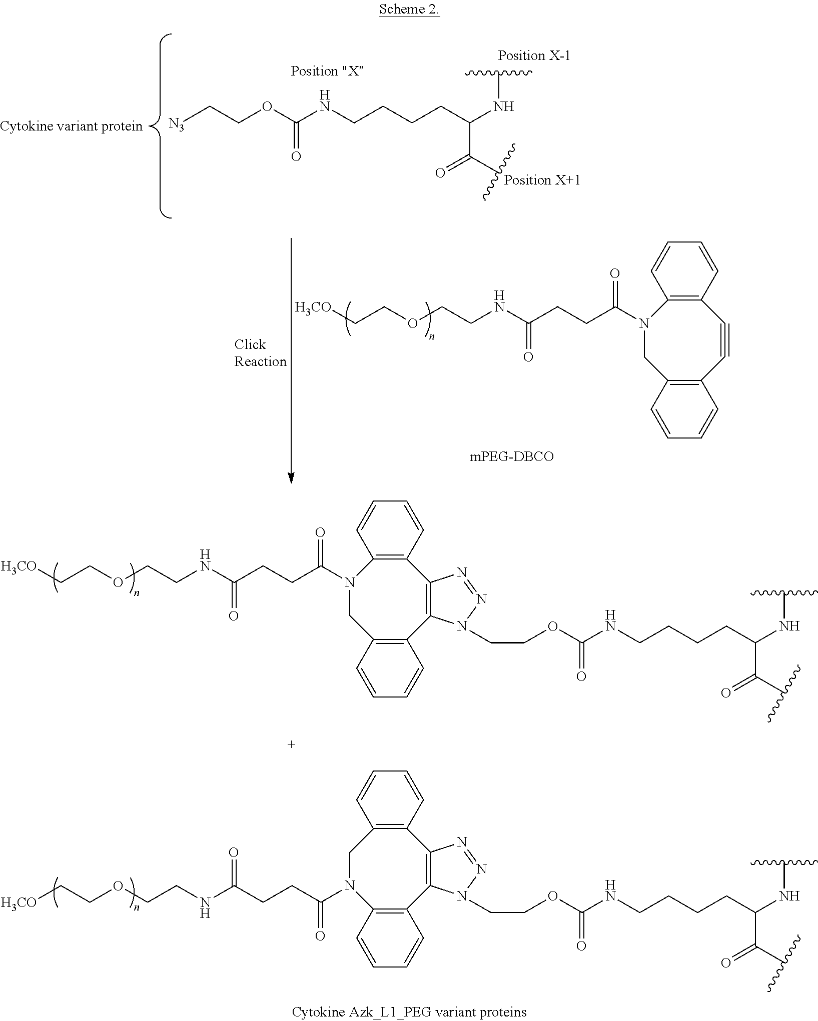

### Example 2. Clinical Study of Biomarker Effects Following IL-2 Conjugate and Pembrolizumab Administration

A study was performed to characterize immunological effects of in vivo administration of an IL-2 conjugate described herein in combination with pembrolizumab. The IL-2 conjugate comprised SEQ ID NO: 2, wherein position 64 is AzK_L1_PEG30 kD, where AzK_L1_PEG30 kD is defined as a structure of Formula (IV) or Formula (V), or a mixture of Formula (IV) and Formula (V), and a 30 kDa, linear mPEG chain. This IL-2 conjugate can also be described as an IL-2 conjugate comprising SEQ ID NO: 1, wherein position 64 is replaced by the structure of Formula (IV) or Formula (V), or a mixture of Formula (IV) and Formula (V), and a 30 kDa, linear mPEG chain. The IL-2 conjugate can also be described as an IL-2 conjugate comprising SEQ ID NO: 1, wherein position 64 is replaced by the structure of Formula (XII) or Formula (XIII), or a mixture of Formula (XII) and Formula (XIII), and a 30 kDa, linear mPEG chain. The compound was prepared as described in Example 1, i.e., using methods wherein a protein was first prepared having SEQ ID NO: 1 in which the proline at position 64 was replaced by N6-((2-azidoethoxy)-carbonyl)-L-lysine AzK. The AzK-containing protein was then allowed to react under click chemistry conditions with DBCO comprising a methoxy, linear PEG group having an average molecular weight of 30 kDa, followed by purification and formulation employing standard procedures.

The IL-2 conjugate and pembrolizumab were administered via IV infusion for 30 minutes every 3 weeks [Q3W]. Effects on the following biomarkers were analyzed as surrogate predictors of safety and/or efficacy:
Eosinophilia (elevated peripheral eosinophil count): Cell surrogate marker for IL-2-induced proliferation of cells (eosinophils) linked to vascular leak syndrome (VLS);
Interleukin 5 (IL-5): Cytokine surrogate marker for IL-2 induced activation of type 2 innate lymphoid cells and release of this chemoattractant that leads to eosinophilia and potentially VLS;
Interleukin 6 (IL-6): Cytokine surrogate marker for IL-2 induced cytokine release syndrome (CRS); and
Interferon γ (IFN-γ): Cytokine surrogate marker for IL-2 induced activation of CD8+ cytotoxic T lymphocytes and NK cells.

Effects on the cell counts of the following biomarkers were analyzed as surrogate predictors of anti-tumor immune activity:
Peripheral CD8+ Effector Cells: Marker for IL-2-induced proliferation of these target cells in the periphery that upon infiltration become a surrogate marker of inducing a potentially latent therapeutic response;
Peripheral CD8+ Memory Cells: Marker for IL-2-induced proliferation of these target cells in the periphery that upon infiltration become a surrogate marker of inducing a potentially durable latent therapeutic and maintenance of the memory population;
Peripheral NK Cells: Marker for IL-2-induced proliferation of these target cells in the periphery that upon infiltration become a surrogate marker of inducing a potentially rapid therapeutic response; and
Peripheral CD4+ Regulatory Cells: Marker for IL-2-induced proliferation of these target cells in the periphery that upon infiltration become a surrogate marker of inducing an immunosuppressive TME and offsetting of an effector-based therapeutic effect.

Subjects were human males or females aged ≥18 years at screening. All subjects had been previously treated with an anti-cancer therapy and met at least one of the following: Treatment related toxicity resolved to grade 0 or 1 (alopecia excepted) according to NCI CTCAE v5.0; or Treatment related toxicity resolved to at least grade 2 according to NCI CTCAE v5.0 with prior approval of the Medical Monitor. The most common tumors included cervical cancer, head and neck squamous cell carcinoma, basal cell carcinoma, melanoma and non-small cell lung cancer.

Subjects also met the following criteria: Provided informed consent. Eastern Cooperative Oncology Group (ECOG) performance status of 0 or 1. Life expectancy greater than or equal to 12 weeks as determined by the Investigator. Histologically or cytologically confirmed diagnosis of advanced and/or metastatic solid tumors. Subjects with advanced or metastatic solid tumors who have refused standard of care; or for whom no reasonable standard of care exists that would confer clinical benefit; or for whom standard therapy is intolerable, not effective, or not accessible. Measurable disease per RECIST v1.1. Adequate laboratory parameters including: Absolute lymphocyte count ≥0.5 times lower limit of normal; Platelet count ≥100×10⁹/L; Hemoglobin ≥9.0 g/dL (absence of growth factors or transfusions within 2 weeks; 1-week washout for ESA and CSF administration is sufficient); Absolute neutrophil count ≥1.5×10⁹/L (absence of growth factors within 2 weeks); Prothrombin time (PT) and partial thromboplastin time (PTT)≤1.5 times upper limit of normal (ULN); Aspartate aminotransferase (AST) and alanine aminotransferase (ALT)≤2.5 times ULN except if liver metastases are present may be ≤5 times ULN; Total bilirubin ≤1.5×ULN. Premenopausal women and women less than 12 months after menopause had a negative serum pregnancy test within 7 days prior to initiating study treatment.

Cohorts Treated with 8 μg/Kg and 16 μg/Kg Doses

Q3W dosing. 10 adults (6 [60%] male, 4 [40%] female, 9 [90%] Caucasian) having advanced or metastatic solid tumors and whose age ranged from 42-70 years received a) the IL-2 conjugate at an 8 μg/kg dose IV Q3W or 16 μg/kg dose IV Q3W and b) pembrolizumab at a dose of 200 mg IV Q3W sequentially for at least one cycle. Here and throughout Example 2, drug mass per kg subject (e.g., 8 μg/kg) refers to IL-2 mass exclusive of PEG and linker mass. The results below are for subjects receiving an 8 μg/kg dose IV Q3W and pembrolizumab (4 subjects) or 16 μg/kg dose IV Q3W and pembrolizumab (6 subjects), who received treatment for 2-19 cycles.

Two subjects who received 8 μg/kg IL-2 conjugate and pembrolizumab had confirmed partial responses (PRs; 1 PD-1-naïve basal cell carcinoma, 1 head and neck squamous cell carcinoma, who had received prior anti-PD-1) ongoing for 9+ months. One subject (non-small cell lung cancer) who received 16 μg/kg IL-2 conjugate and pembrolizumab had disease stabilization for about 6 months. Six subjects had disease progression (at the 6-week assessment); one subject had initial disease stabilization (at the 6 week assessment; followed by progressive disease). The four subjects receiving 8 μg/kg IL-2 conjugate and pembrolizumab had increased post-dose CD8+Ki67 expression levels (15%-70%).

One 59 year old male with head and neck squamous cell carcinoma receiving 8 μg/kg IL-2 conjugate and pembrolizumab received 18 cycles and had a confirmed partial response (39% decrease after 8 cycles; 47% decrease after 11 cycles). This subject had previously received 4 lines of systemic therapy including 2 anti-PD1 treatments; the best response to an anti-PD1 treatment had been stable disease.

One 50 year old male with basal cell carcinoma receiving 8 μg/kg IL-2 conjugate and pembrolizumab received 17 cycles and had a confirmed partial response (50% decrease after 2 cycles, and 80% decrease after 8 cycles). This subject had previously undergone surgeries and radiation therapy.

The maximal tumor responses in other patients with immune sensitive tumors were found to be melanoma (23% and 11% growth), basal cell carcinoma (4% growth), and non-small cell lung cancer (18% reduction).

The peak peripheral expansion of CD8+ T effector cells averaged 2.06-fold above baseline in subjects receiving 8 μg/kg IL-2 conjugate and pembrolizumab. All four subjects had post-dose NK Cell Ki67 expression levels of nearly 100 percent. The subjects had post-dose peak peripheral expansion of NK cells that averaged 6.73-fold above baseline at day 3. The peak peripheral expansion of CD8+ T effector cells averaged 3.71-fold above baseline in subjects receiving 16 μg/kg IL-2 conjugate and pembrolizumab.

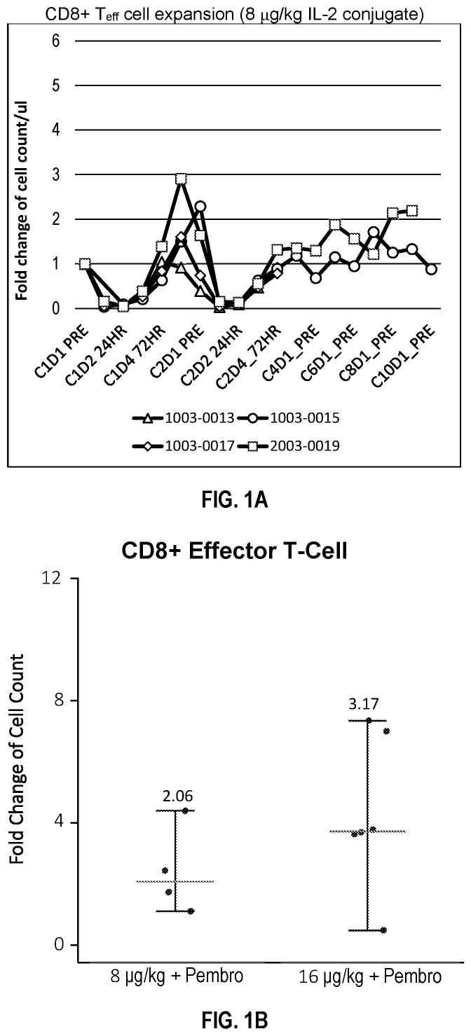

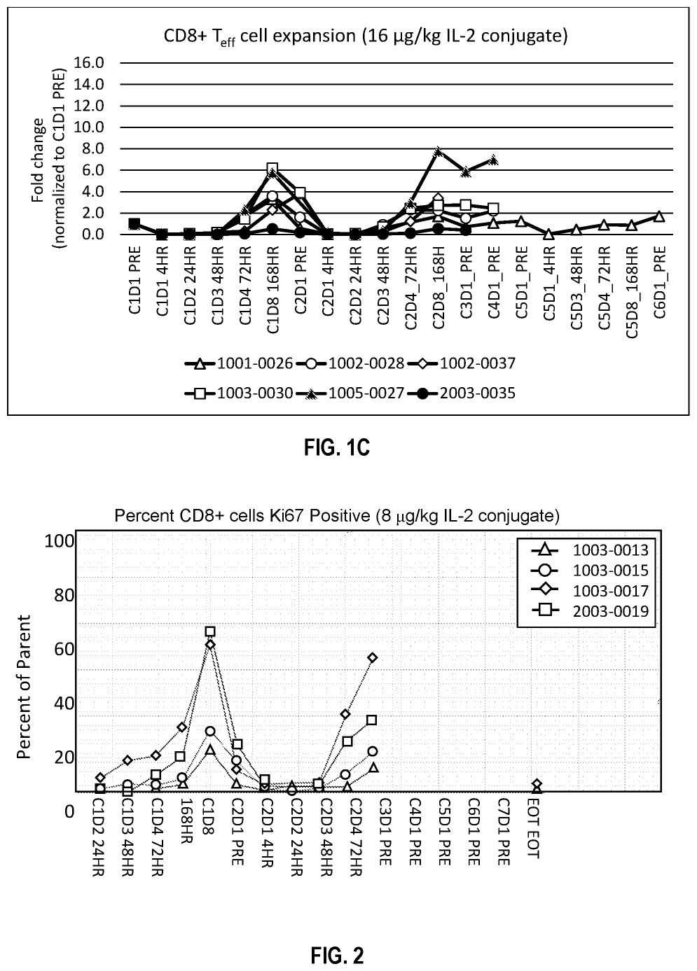

Efficacy biomarkers. Peripheral CD8+ T_{eff} cell counts were measured (FIGS. 1A-C). Prolonged CD8+ expansion over baseline (e.g., greater than or equal to 1.5-fold change) was observed at 3 weeks after the previous dose in some subjects. The percentage of CD8+ T_{eff} cells expressing Ki67 was also measured (FIG. 2).

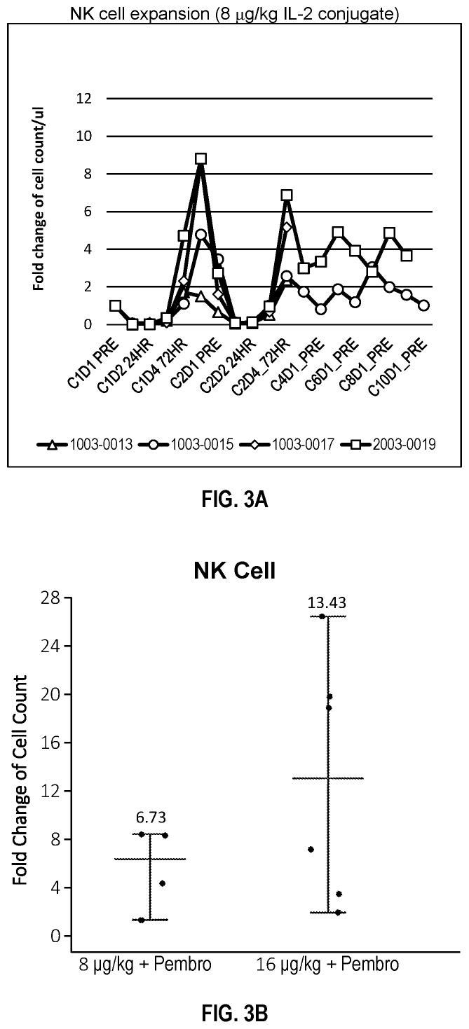

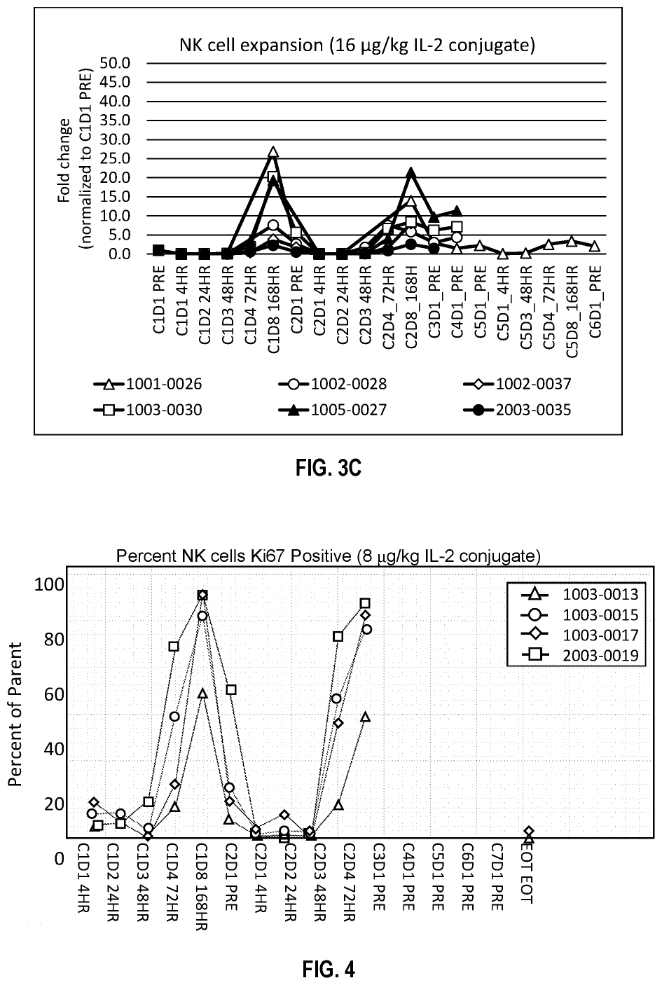

Peripheral NK cell counts are shown in FIGS. 3A-C. Prolonged NK cell expansion over baseline (e.g., greater than or equal to 2-fold change) was observed at 3 weeks after the previous dose in some subjects. The percentage of NK cells expressing Ki67 was also measured (FIG. 4).

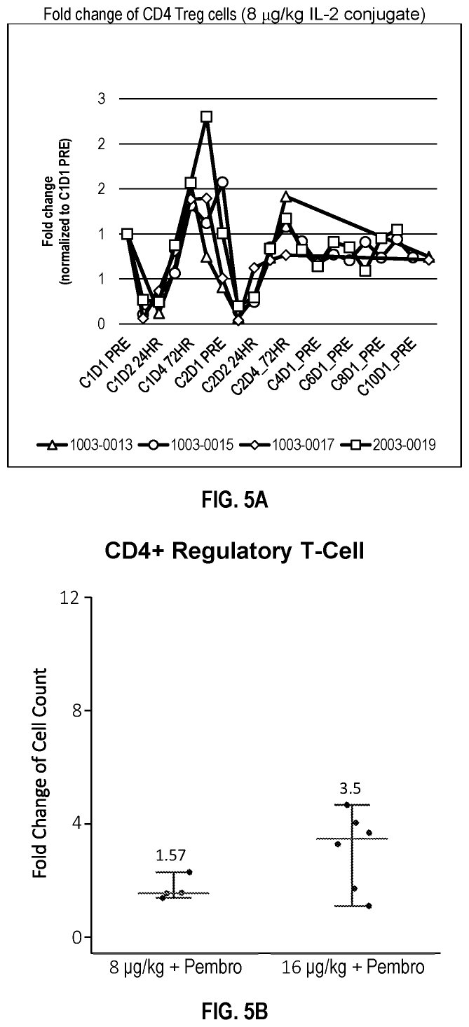

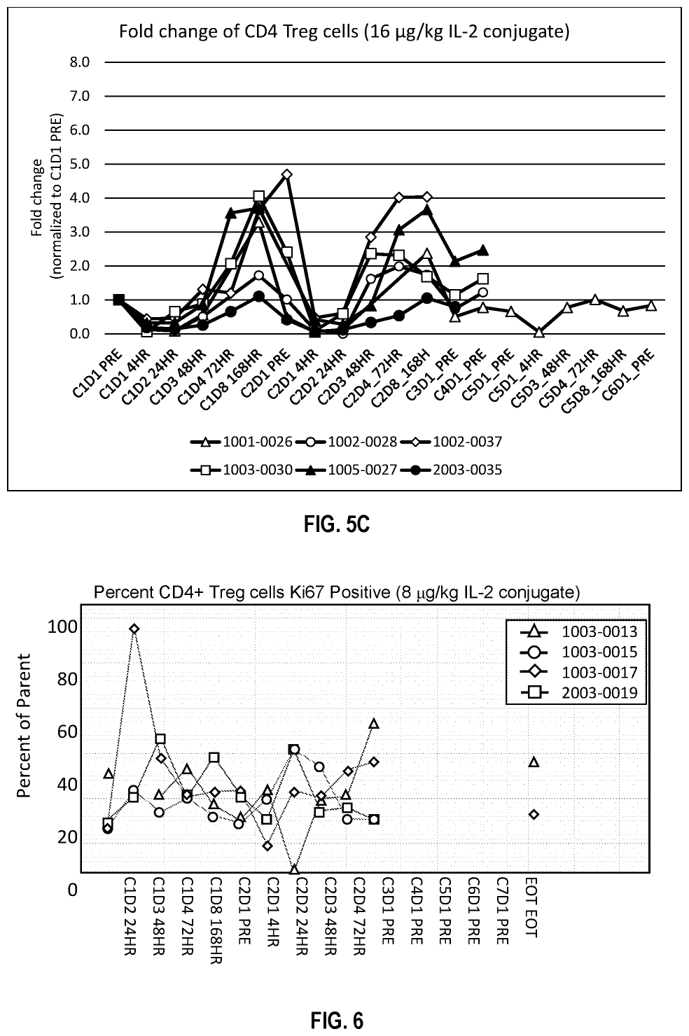

Peripheral CD4+ T_{reg} counts are shown in FIGS. 5A-C. The percentage of CD4+ T_{reg} cells expressing Ki67 was also measured (FIG. 6).

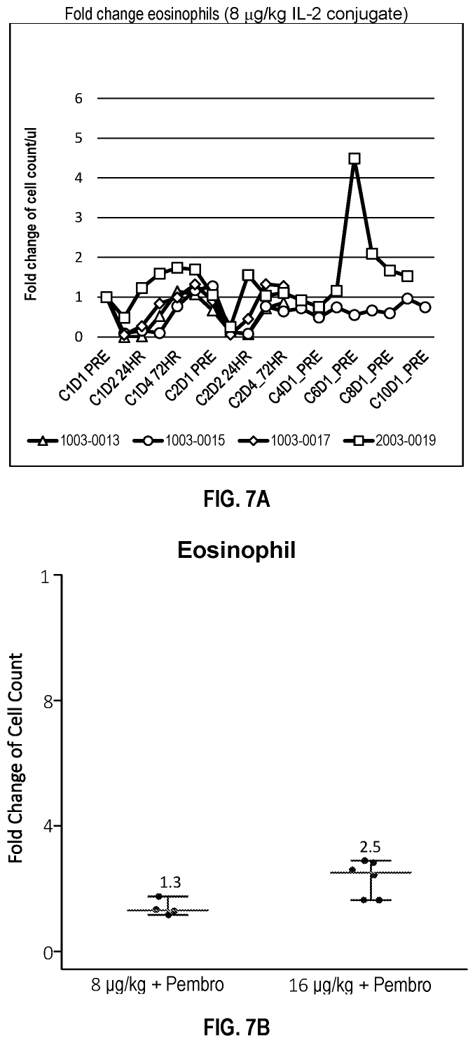

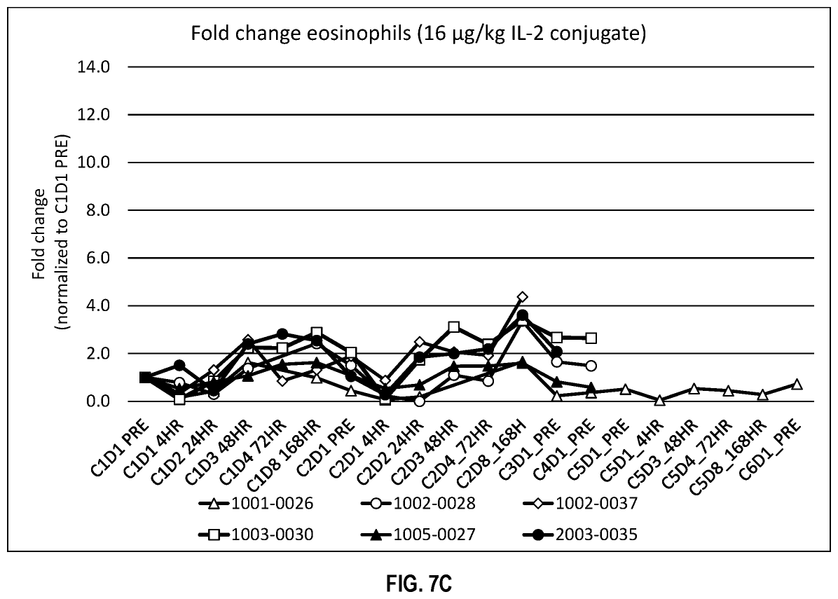

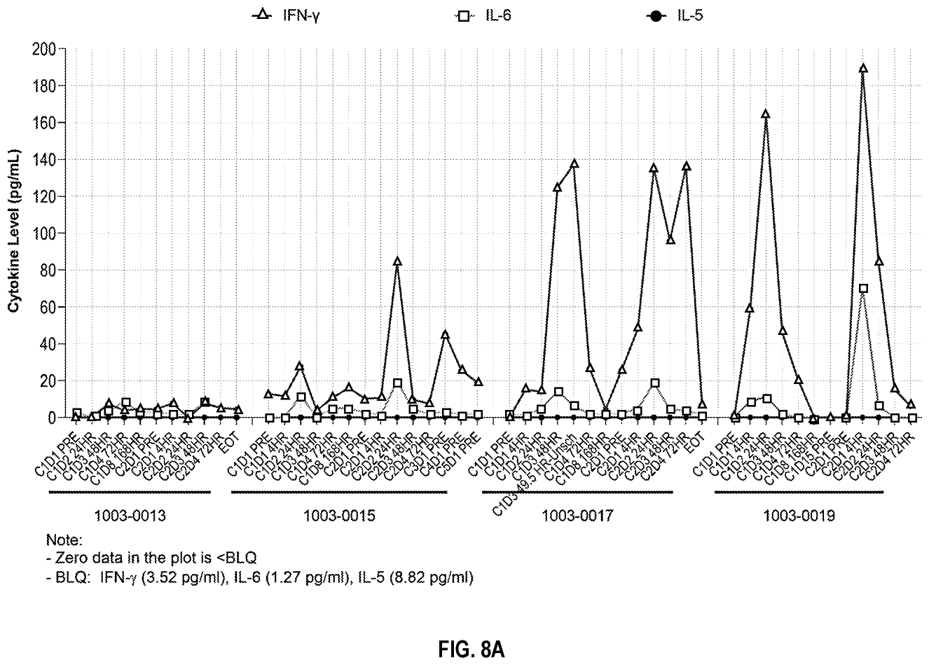

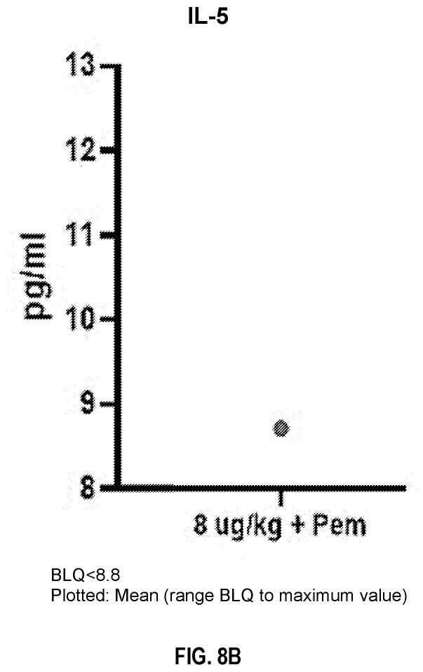

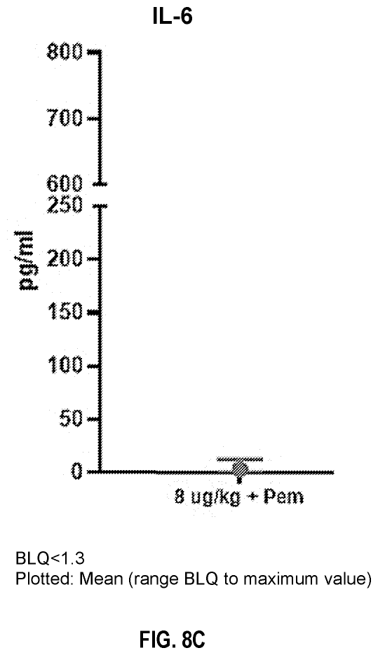

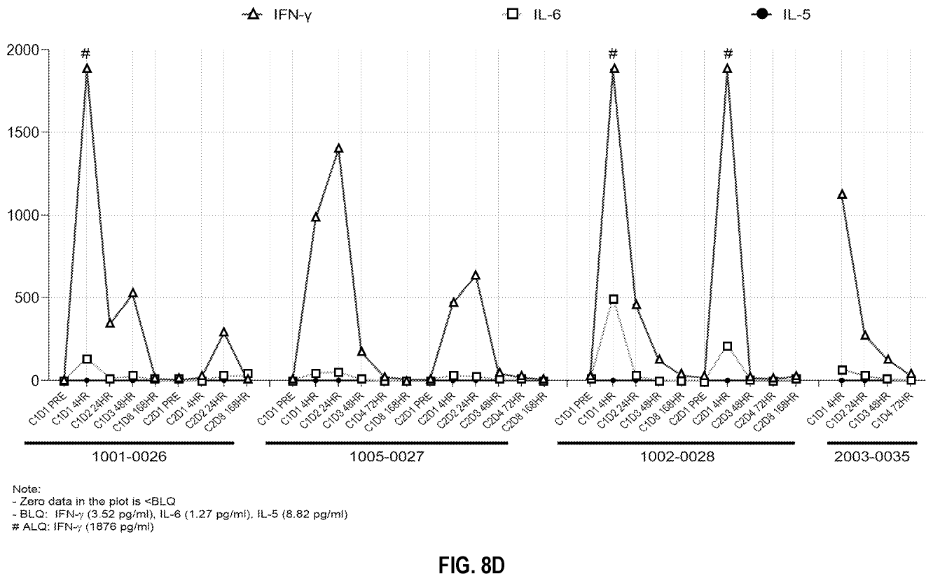

Eosinophil counts were measured (FIGS. 7A-C). The measured values were consistently below the range of 2328-15958 eosinophils/μL in patients with IL-2 induced eosinophilia as reported in Pisani et al., Blood 1991 Sep. 15; 78(6):1538-44. Levels of IFN-γ, IL-5, and IL-6 were also measured (FIGS. 8A-D). The measured values show that IFN-γ was induced, but low amounts of IL-5 and IL-6, cytokines associated with VLS and CRS, respectively, were induced.

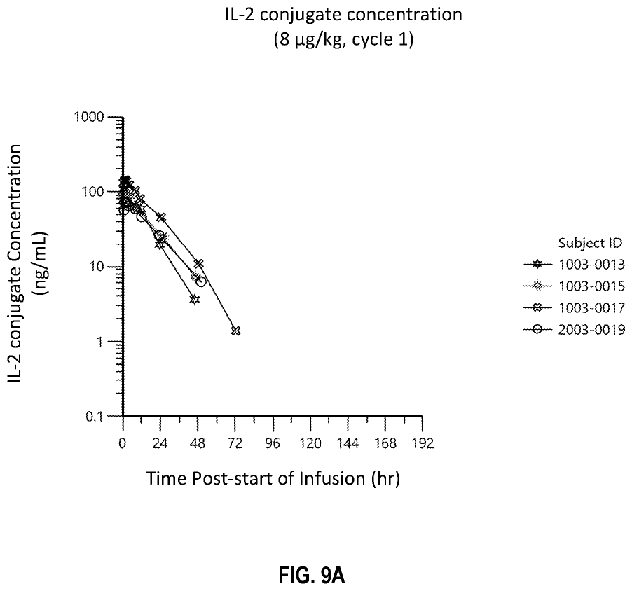

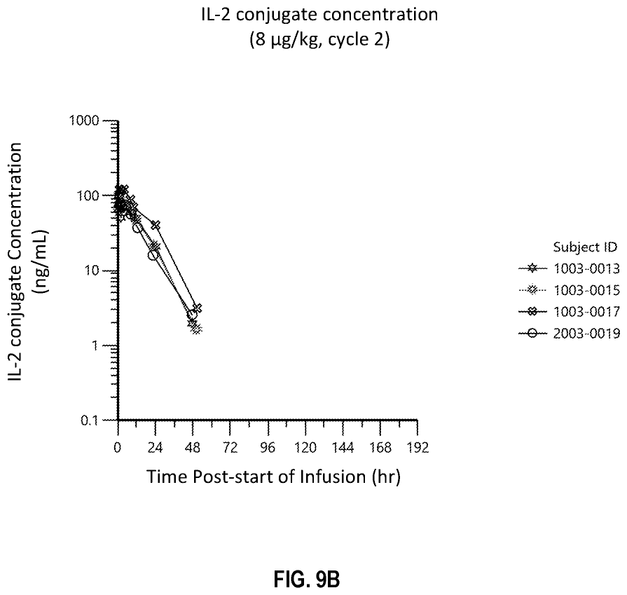

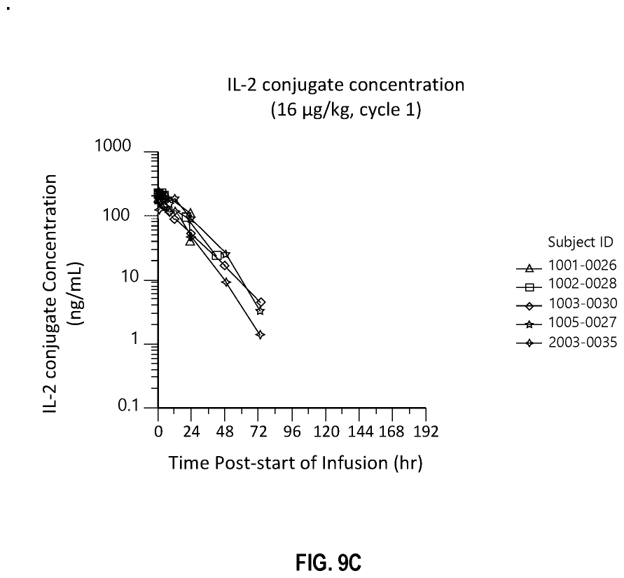

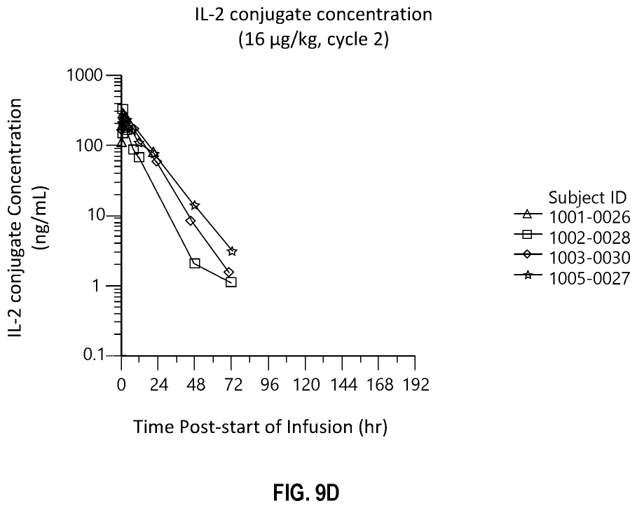

Mean concentrations of the IL-2 conjugate, administered at a dose of 8 μg/kg, after 1 and 2 cycles are shown in FIG. 9A and FIG. 9B, respectively. Mean concentrations of the IL-2 conjugate, administered at a dose of 16 μg/kg, after 1 and 2 cycles are shown in FIG. 9C and FIG. 9D, respectively.

Anti-drug Antibodies (ADAs). Samples from treated subjects were assayed after each dose cycle for anti-drug antibodies (ADAs). Anti-polyethylene glycol autoantibodies were detected by direct immunoassays (detection limit: 36 ng/mL). A bridging MesoScale Discovery ELISA was performed with a labeled form of the IL-2 conjugate, having a detection limit of 4.66 ng/mL. Additionally, a cell-based assay for neutralizing antibodies against the IL-2 conjugate was performed using the CTLL-2 cell line, with STATS phosphorylation as the readout (detection limit: 6.3 μg/mL).

Samples were collected and analyzed after each dose cycle from four subjects where 2 patients received 2 cycles and the other two patients received 10 or 11 cycles. An assay-specific cut point was determined during assay qualification as a signal to negative ratio of 1.09 or higher for the IL-2 conjugate ADA assay and 2.08 for the PEG ADA assay. Samples that gave positive or inconclusive results in the IL-2 conjugate assay were subjected to confirmatory testing in which samples and controls were assayed in the presence and absence of confirmatory buffer (10 μg/mL IL-2 conjugate in blocking solution). Samples that gave positive or inconclusive results in the PEG assay were subjected to confirmatory testing in which samples and controls were assayed in the presence and absence of confirmatory buffer (10 μg/mL IL-2 conjugate in 6% horse serum). Samples will be considered “confirmed” if their absorbance signal is inhibited by equal to or greater than an assay-specific cut point determined during assay qualification (14.5% for the IL-2 conjugate or 42.4% for PEG) in the detection step. No confirmed ADA against the IL-2 conjugate or PEG were detected (data not shown).

Summary of Results; Discussion. All subjects had elevated post-dose CD8+ Ki67 expression levels (FIG. 2), with peripheral expansion of CD8+ T effector (Teff) cells averaging 1.95-fold above baseline. All 4 subjects also had elevated post-dose NK cell Ki67 expression levels (FIG. 4), with peripheral expansion of NK cells averaging 6.73-fold above baseline at day 3. There were no meaningful elevations in IL-5 and IL-6 levels.

An AE was any untoward medical occurrence in a clinical investigation subject administered a pharmaceutical product, regardless of causal attribution. Dose-limiting toxicities were defined as an AE occurring within Day 1 through Day 29 (inclusive)±1 day of a treatment cycle that was not clearly or incontrovertibly solely related to an extraneous cause and that met at least one of the following criteria:
Grade 3 neutropenia (absolute neutrophil count <1000/mm³>500/mm³) lasting ≥7 days, or Grade 4 neutropenia of any duration
Grade 3+ febrile neutropenia
Grade 4+ thrombocytopenia (platelet count <25,000/mm³)
Grade 3+ thrombocytopenia (platelet count <50,000-25,000/mm³) lasting ≥5 days, or associated with clinically significant bleeding or requiring platelet transfusion
Failure to meet recovery criteria of an absolute neutrophil count of at least 1,000 cells/mm³ and a platelet count of at least 75,000 cells/mm³ within 10 days
Any other grade 4+ hematologic toxicity lasting ≥5 days
Grade 3+ ALT or AST in combination with a bilirubin >2 times ULN with no evidence of cholestasis or another cause such as viral infection or other drugs (i.e. Hy's law)
Grade 3 infusion-related reaction that occurs with premedication; Grade 4 infusion-related reaction
Grade 3 Vascular Leak Syndrome defined as hypotension associated with fluid retention and pulmonary edema
Grade 3+ anaphylaxis
Grade 3+ hypotension
Grade 3+ AE that does not resolve to grade <2 within 7 days of starting accepted standard of care medical management
Grade 3+ cytokine release syndrome

The following exceptions applied to non-hematologic AEs:
Grade 3 fatigue, nausea, vomiting, or diarrhea that resolves to grade ≤2 with optimal medical management in ≤3 days
Grade 3 fever (as defined by >40° C. for ≤24 hours)
Grade 3 infusion-related reaction that occurs without premedication; subsequent doses should use premedication and if reaction recurs then it will be a DLT
Grade 3 arthralgia or rash that resolves to grade ≤2 within 7 days of starting accepted standard of care medical management (e.g. systemic corticosteroid therapy)
   If a subject had grade 1 or 2 ALT or AST elevation at baseline considered secondhand to liver metastases, a grade 3 elevation must also be ≥3 times baseline and last >7 days.

Serious AEs were defined as any AE that results in any of the following outcomes: Death; Life-threatening AE; Inpatient hospitalization or prolongation of an existing hospitalization; A persistent or significant incapacity or substantial disruption of the ability to conduct normal life functions; or a congenital anomaly/birth defect. Important medical events that may not result in death, be life-threatening, or require hospitalization may be considered serious when, based upon appropriate medical judgment, they may jeopardize the subject and may require medical or surgical intervention to prevent one of the outcomes listed above. Examples of such medical events include allergic bronchospasm requiring intensive treatment in an emergency room or at home, blood dyscrasias or convulsions that do not result in inpatient hospitalization, or the development of drug dependency or drug abuse.

There were no dose-limiting toxicities reported at either dose and there were no treatment-related adverse events (TRAE) leading to discontinuation. Two TRAEs led to dosage reduction. There were 5 treatment-related serious AEs reported in three of the six patients treated at 16 μg/kg dose IV Q3W.

At least 9 subjects experienced TRAEs. The most common TRAEs (>2 patients) of all grades by SOC included general disorders and administration conditions (9/10), investigations (6/10 subjects), metabolism and nutrition (4/10), nervous system disorders (4/10), respiratory, thoracic and mediastinal disorders (4/10), vascular disorders (3/10), skin and subcutaneous disorders (3/10), blood and lymphatic disorders, cardiac disorders, gastrointestinal disorders, immune system disorders, infections and infestations, and musculoskeletal (2/10). TEAEs by preferred terms are detailed in Table 1.

TABLE 1

| Adverse Events (PT), n (%) | Frequency (N = 10) |
|---|---|
| Anemia | 2 (20%) |
| Influenza-Like Illness | 4 (40%) |
| Pyrexia | 6 (60%) |
| Chills | 4 (40%) |
| Fatigue | 5 (50%) |
| Nausea | 2 (20%) |
| Vomiting | 2 (20%) |
| ALT increase | 4 (40%) |
| AST Increase | 4 (40%) |
| Decreased Appetite | 1 (10%) |
| Hypophosphatemia | 3 (30%) |
| Lymphocyte Count Decreased | 2 (20%) |
| Hypotension | 3 (30%) |

Treatment-related AEs were transient and resolved with accepted standard of care. AEs of fever, hypotension, and hypoxia did not correlate with IL-5/IL-6 cytokine elevation. No cumulative toxicity, end organ toxicity, vascular leak syndrome, or eosinophilia was observed. IL-5 levels remained at or below the lowest level of detection. One subject had G2 hypotension which resolved with hydration. One subject had G3 cytokine release syndrome (fever+hypotension requiring pressors; subject had baseline orthostatic hypotension) resulting in dose reduction. There was no notable impact to vital signs, no QTc prolongation, or other cardiac toxicity. Accordingly, the IL-2 conjugate in combination with pembrolizumab demonstrated encouraging PD data and was generally well-tolerated with no discontinuations due to TRAE. It was determined that the in vivo half-life of the IL-2 conjugate was about 10 hours. Overall, the results are considered to support non-alpha preferential activity of the IL-2 conjugate, with a tolerable safety profile in combination with pembrolizumab as well as encouraging PD and preliminary evidence of activity in patients with immune-sensitive tumors.

Cohort Treated with 24 μg/Kg Dose

Six individuals (male [100%], 4 [66.7%] caucasian) with a median age of 51.5 years, ranging from 46-66 years of age, having advanced or metastatic solid tumors received the IL-2 conjugate at a 24 μg/kg dose Q3W. Tumor types included lung cancer, basal cell carcinoma, and colon cancer.

Each subject was treated with a) the IL-2 conjugate administered via IV infusion at a dose of 24 μg/kg for 30 minutes, and b) pembrolizumab administered at a dose of 200 mg IV sequentially. Treatment was given every 3 weeks [Q3W]. Effects on the same biomarkers described above for the 8 μg/kg and 16 μg/kg doses of the IL-2 conjugate were analyzed as surrogate predictors of safety and/or efficacy. Subjects in these studies met the same criteria as the subjects treated 8 μg/kg and 16 μg/kg doses.

Five (83.3%) of 6 subjects experienced at least one TEAE, and 4 (66.7%) of 6 subjects experienced at least 1 Grade 3-4 related TEAEs (1 Grade 3 and 3 Grade 4). There was one Grade 3 ALT/AST elevation (also with Grade 3 hypophosphatemia) and 3 Grade 4 lymphocyte count decrease (one in a subject with Grade 3 AST/ALT elevation, Grade 2 hyperbilirubinemia-DLT along with Grade 2 CRS). The lymphocyte count recovered to at least Grade 3 in 48 hours.

Two subjects experienced related SAEs: one Grade 1 fever in a subject with adrenal insufficiency requiring steroid adjustment, and one Grade 2 cytokine release syndrome (fever and hypotension requiring fluids and dexamethasone) associated with Grade 3 AST/ALT elevation and G2 hyperbilirubinemia. There was one instance of a DLT: a subject with Grade 3 AST/ALT elevation along with Grade 2 hyperbilirubinemia associated with Grade 2 CRS (fever and hypotension requiring hydration and dexamethasone). For this subject, the dose was reduced for C2D1. No drug discontinuations resulted from TEAEs. TEAEs are detailed in Table 2.

TABLE 2
Treatment Emergent Adverse Events (TEAE) (n = 6)

| System Organ Class | Grade 1 | | Grade 2 | | Grade 3 | | Grade 4 | Grade 5 |
|---|---|---|---|---|---|---|---|---|
| Blood and lymphatic disorders | 0/6 | (0%) | 1/6 | (16.7%) | 1/6 | (16.7%) | 0/6 (0%) | 0/6 (0%) |
| Cardiac disorders | 1/6 | (16.7%) | 0/6 | (0%) | 0/6 | (0%) | 0/6 (0%) | 0/6 (0%) |
| General disorders and | 2/6 | (33.3%) | 2/6 | (33.3%) | 0/6 | (0%) | 0/6 (0%) | 0/6 (0%) |
| administration site conitions | | | | | | | | |
| Gastrointestinal disorders | 3/6 | (50%) | 0/6 | (0%) | 0/6 | (0%) | 0/6 (0%) | 0/6 (0%) |
| Hepatobiliary Disorders | 0/6 | (0%) | 1/6 | (16.7%) | 0/6 | (0%) | 0/6 (0%) | 0/6 (0%) |
| Immune system disorders | 0/6 | (0%) | 1/6 | (16.7%) | 0/6 | (0%) | 0/6 (0%) | 0/6 (0%) |
| Investigations | 0/6 | (0%) | 0/6 | (0%) | 1/6 | (16.7%) | 3/6 (50%) | 0/6 (0%) |
| Metabolism and nutrition | 1/6 | (16.7%) | 2/6 | (33.3%) | 1/6 | (16.7%) | 0/6 (0%) | 0/6 (0%) |
| disorders | | | | | | | | |
| Musculoskeletal and | 1/6 | (16.7%) | 2/6 | (33.3%) | 0/6 | (0%) | 0/6 (0%) | 0/6 (0%) |
| connective tissue disorders | | | | | | | | |
| Psychiatric disorders | 0/6 | (0%) | 1/6 | (16.7%) | 0/6 | (0%) | 0/6 (0%) | 0/6 (0%) |
| Respiratory, thoracic and | 1/6 | (16.7%) | 0/6 | (0%) | 1/6 | (16.7%) | 0/6 (0%) | 0/6 (0%) |
| mediastinal disorders | | | | | | | | |
| Skin and subcutaneous | 1/6 | (16.7%) | 0/6 | (0%) | 0/6 | (0%) | 0/6 (0%) | 0/6 (0%) |
| disorders | | | | | | | | |
| Vascular disorders | 1/6 | (16.7%) | 0/6 | (0%) | 0/6 | (0%) | 0/6 (0%) | 0/6 (0%) |

The following related events were reported: one Grade 3 AST/ALT and Grade 2 bilirubin (DLT) in the setting of Grade 2 CRS (fever, hypotension [BP97/56 mm Hg] and hypoxemia [SpO2 92%]) managed with fluid bolus, supplemental oxygen and dexamethasone with resolution required a dose reduction for C2D1; one patient fever, chills, rigors and hypoxemia (92%) requiring supportive care and oxygen (C2D1); one Grade 3 AST/ALT (C2D8) presumed related to IL-2 conjugate and pembrolizumab without other symptoms in the setting of alcoholism; and three Grade 4 lymphocyte count decrease.

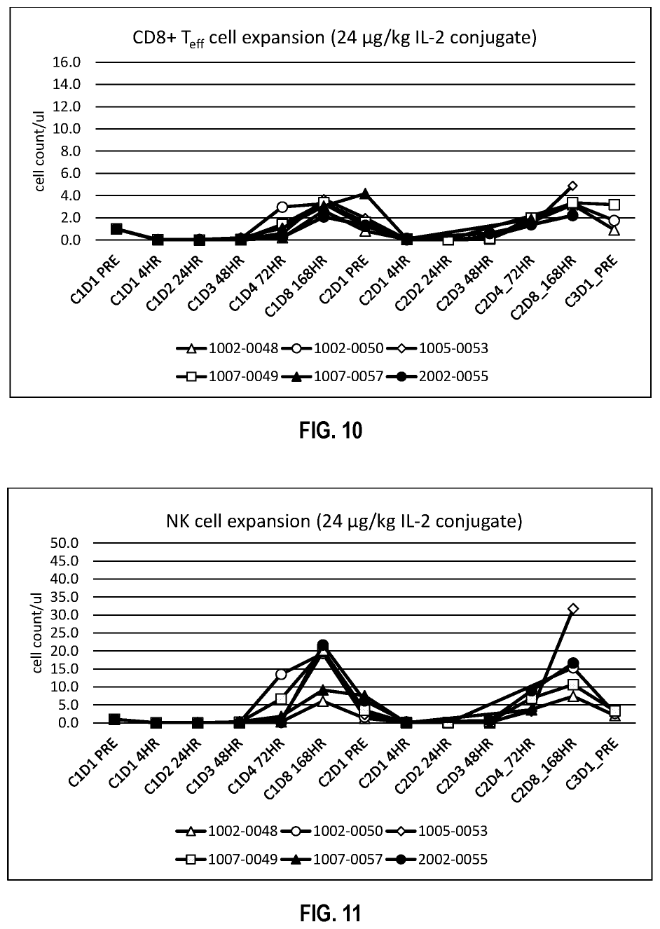

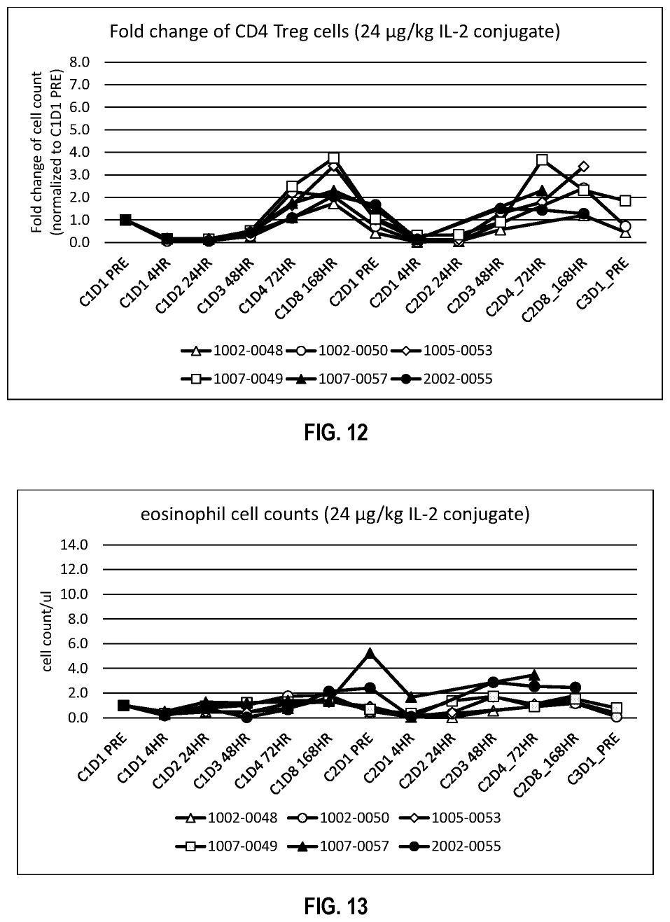

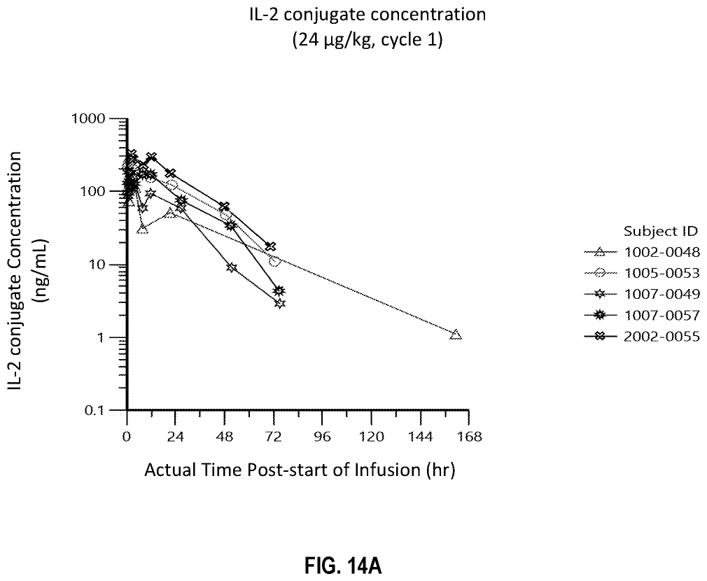

Efficacy biomarkers. Peripheral CD8+ T_{eff} cell counts were measured (FIG. 10), and peripheral NK cell counts are shown in FIG. 11. Peripheral CD4+ T_{reg} cell counts are shown in FIG. 12, and peripheral eosinophil cell counts are shown in FIG. 13.

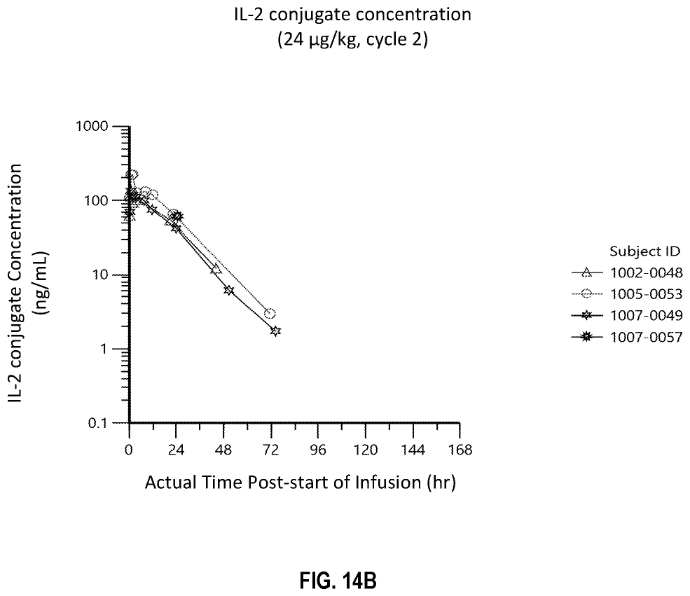

Mean concentrations of the IL-2 conjugate after 1 and 2 cycles are shown in FIG. 14A and FIG. 14B, respectively.

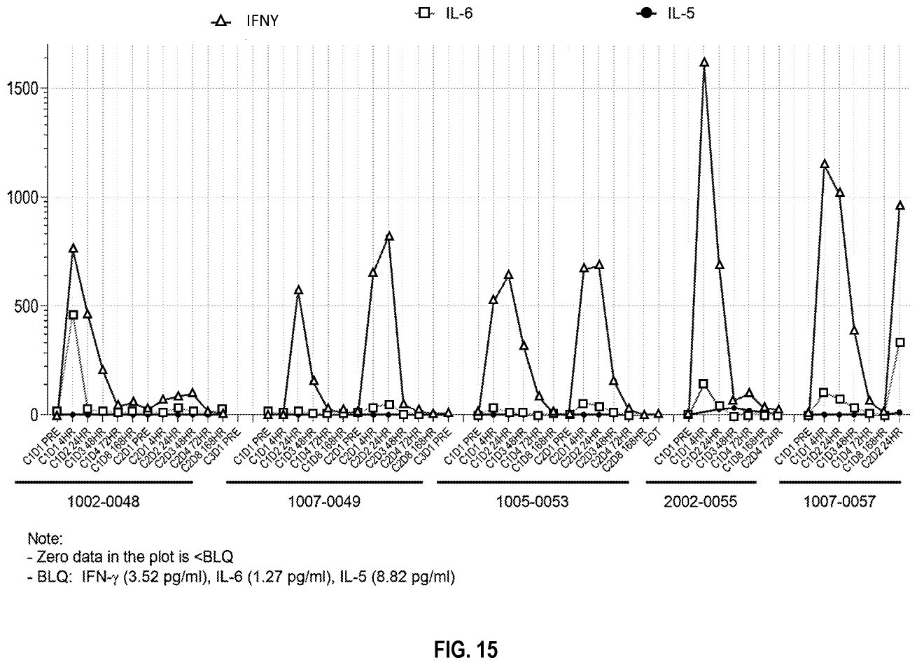

Cytokine levels (IFN-γ, IL-6, and IL-5) are shown in FIG. 15.

Accordingly, the IL-2 conjugate in combination with pembrolizumab demonstrated encouraging PD data and was generally well-tolerated with no discontinuations due to TRAE. Overall, the results are considered to support non-alpha preferential activity of the IL-2 conjugate, with a tolerable safety profile in combination with pembrolizumab as well as encouraging PD and preliminary evidence of activity in patients with immune-sensitive tumors.

Cohort Treated with 32 μg/Kg Dose

Three individuals having advanced or metastatic solid tumors received the IL-2 conjugate at a 32 μg/kg dose Q3W. Tumor types included ovarian carcinoma.

Each subject was treated with a) the IL-2 conjugate administered via IV infusion at a dose of 32 μg/kg for 30 minutes, and b) pembrolizumab administered at a dose of 200 mg IV sequentially. Treatment was given every 3 weeks [Q3W]. Effects on the same biomarkers described above for the 8 μg/kg and 16 μg/kg IL-2 conjugate doses were analyzed as surrogate predictors of safety and/or efficacy. Subjects in these studies met the same criteria as the subjects treated 8 μg/kg and 16 μg/kg doses.

All three (100%) subjects experienced at least one TEAE, and one (33.3%) of 3 subjects experienced at least 1 Grade 3-4 related TEAEs (1 Grade 4). There was one instance of Grade 4 lymphocyte count decrease (subject also had G3 fever). There was one related SAEs of Grade 1 fever and Grade 1 tachycardia requiring hospitalization for 24 hours (C2D2-C2D3). This was resolved with supportive care. There were no DLTs and no drug discontinuations resulting from TEAEs. TEAEs are detailed in Table 3.

TABLE 3
Treatment Emergent Adverse Events (TEAE) (n = 3)

| System Organ Class | Grade 1 | | Grade 2 | | Grade 3 | Grade 4 | Grade 5 |
|---|---|---|---|---|---|---|---|
| Blood and lymphatic disorders | 0/3 | (0%) | 1/3 | (33.3%) | 0/3 (0%) | 0/3 (0%) | 0/3 (0%) |
| Cardiac disorders | 1/3 | (33.3%) | 0/3 | (0%) | 0/3 (0%) | 0/3 (0%) | 0/3 (0%) |
| Endocrine disorders | 0/3 | (0%) | 1/3 | (33.3%) | 0/3 (0%) | 0/3 (0%) | 0/3 (0%) |
| Gastrointestinal disorders | 1/3 | (33.3%) | 1/3 | (33.3%) | 0/3 (0%) | 0/3 (0%) | 0/3 (0%) |
| General disorders and | 2/3 | (66.6%) | 1/3 | (33.3%) | 0/3 (0%) | 0/3 (0%) | 0/3 (0%) |
| administration conditions | | | | | | | |
| Immune system disorders | 0/3 | (0%) | 1/3 | (33.3%) | 0/3 (0%) | 0/3 (0%) | 0/3 (0%) |
| Infections and infestations | 1/3 | (33.3%) | 0/3 | (0%) | 0/3 (0%) | 0/3 (0%) | 0/3 (0%) |
| Investigations | 0/3 | (0%) | 2/3 | (66.6%) | 0/3 (0%) | 1/3 (33.3%) | 0/3 (0%) |
| Metabolism and nutrition | 0/3 | (0%) | 2/3 | (66.6%) | 0/3 (0%) | 0/3 (0%) | 0/3 (0%) |
| disorders | | | | | | | |
| Nervous system disorders | 1/3 | (33.3%) | 0/3 | (0%) | 0/3 (0%) | 0/3 (0%) | 0/3 (0%) |
| Respiratory, thoracic and | 1/3 | (33.3%) | 0/3 | (0%) | 0/3 (0%) | 0/3 (0%) | 0/3 (0%) |
| mediastinal disorders | | | | | | | |
| Skin and subcutaneous | 1/3 | (33.3%) | 0/3 | (0%) | 0/3 (0%) | 0/3 (0%) | 0/3 (0%) |
| disorders | | | | | | | |
| Vascular disorders | 1/3 | (33.3%) | 0/3 | (0%) | 0/3 (0%) | 0/3 (0%) | 0/3 (0%) |

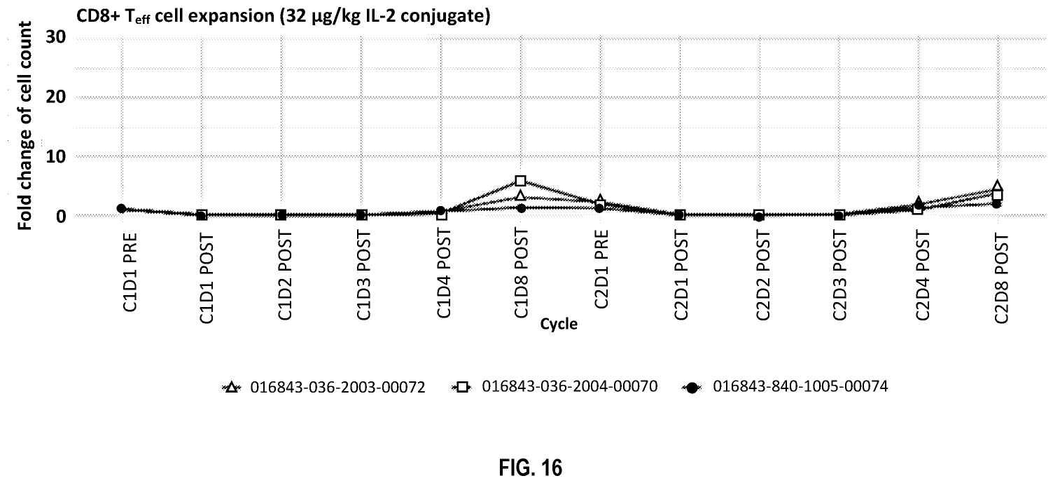

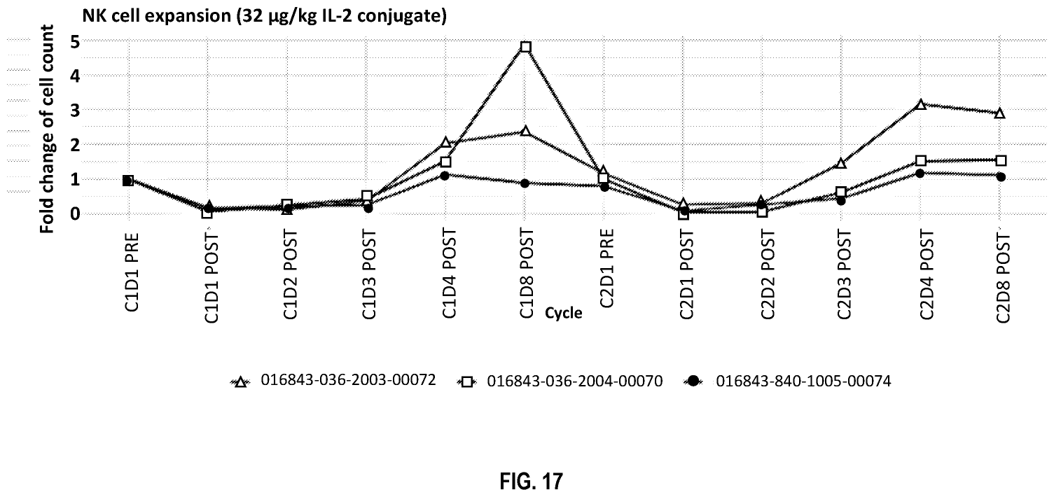

Efficacy biomarkers. Peripheral CD8+ T_{eff} cell counts were measured (FIG. 16). Peripheral CD4+ T_{reg} cell counts are shown in FIG. 17.

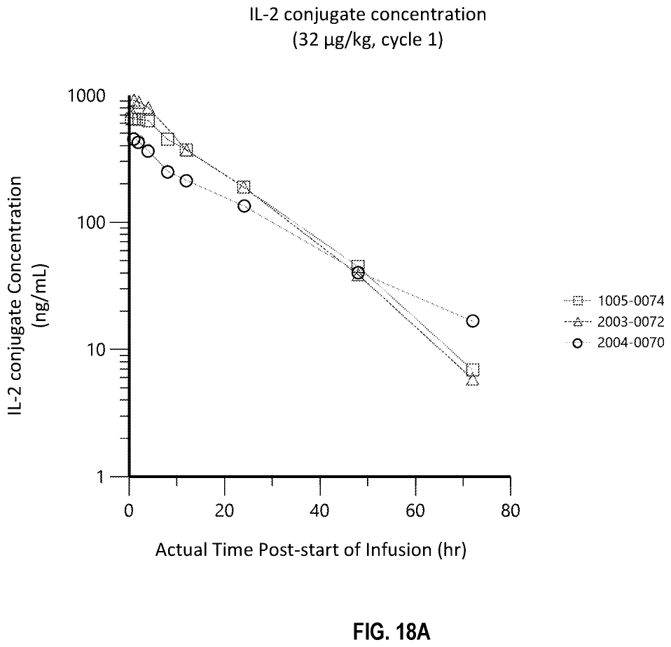

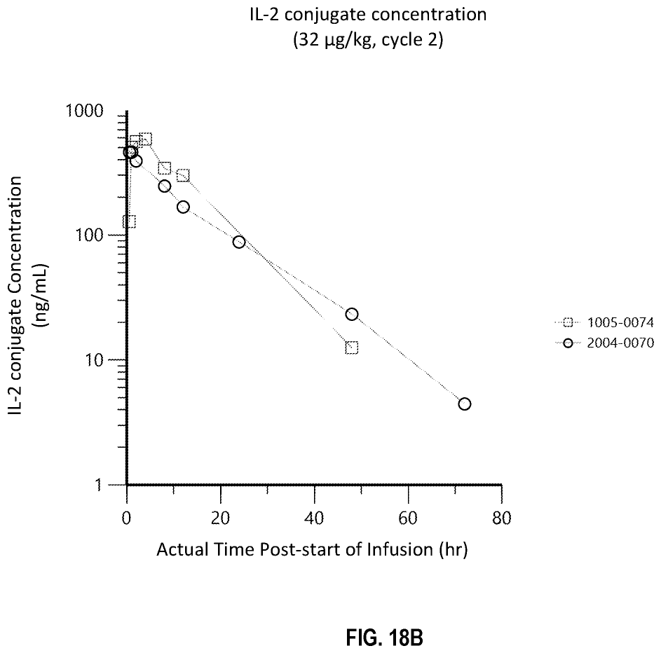

Mean concentrations of the IL-2 conjugate after 1 and 2 cycles are shown in FIG. 18A and FIG. 18B, respectively.

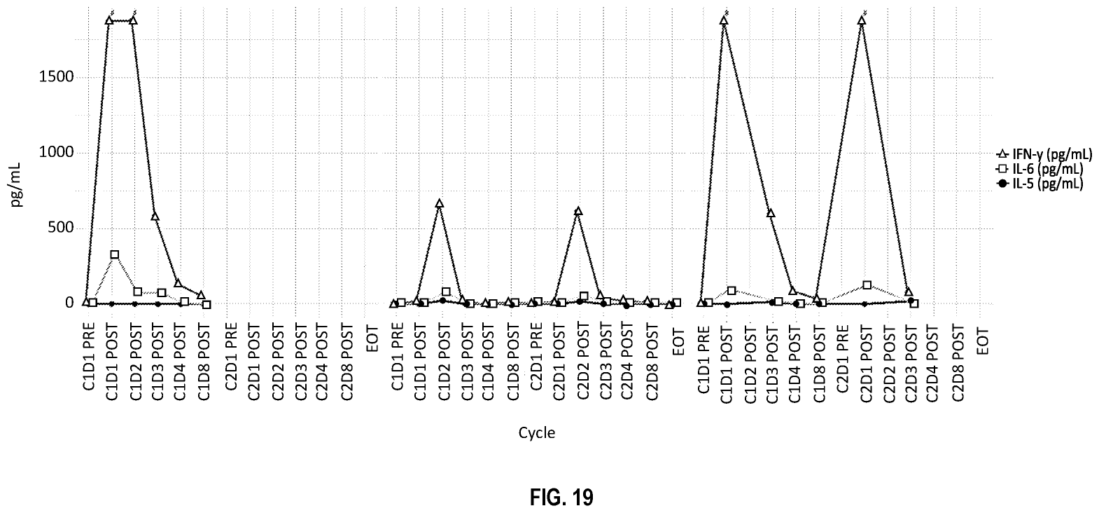

Cytokine levels (IFN-γ, IL-6, and IL-5) are shown in FIG. 19.

Accordingly, the IL-2 conjugate in combination with pembrolizumab demonstrated encouraging PD data and was generally well-tolerated with no discontinuations due to TEAE. Overall, the results are considered to support non-alpha preferential activity of the IL-2 conjugate, with a tolerable safety profile in combination with pembrolizumab as well as encouraging PD and preliminary evidence of activity in patients with immune-sensitive tumors.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. The disclosures of all patent and scientific literature cited herein are expressly incorporated herein in their entirety by reference.

What is claimed is:

1. A method of treating a cancer in a subject in need thereof, comprising administering to the subject (a) about 8 μg/kg, 16 μg/kg, 24 μg/kg, or 32 μg/kg of an IL-2 conjugate, and (b) pembrolizumab, wherein the IL-2 conjugate comprises the amino acid sequence of SEQ ID NO: 1 wherein the amino acid at position P64 is replaced by the structure of Formula (IA): 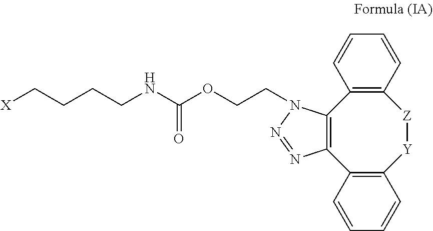
wherein:
Z is CH₂ and Y is 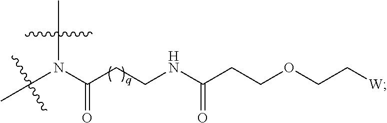
Y is CH₂ and Z is 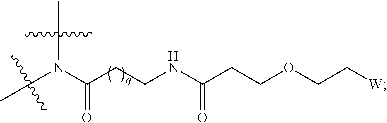
Z is CH₂ and Y is 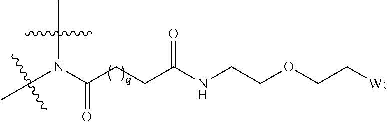
or
Y is CH₂ and Z 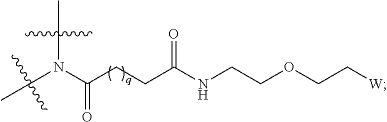
W is a PEG group having an average molecular weight of about 25 kDa-35 kDa;
q is 1, 2, or 3;
X is an L-amino acid having the structure: 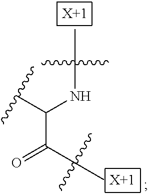
X−1 indicates the point of attachment to the preceding amino acid residue; and
X+1 indicates the point of attachment to the following amino acid residue.

2. An IL-2 conjugate for use in a method of treating a cancer in a subject in need thereof, the method comprising administering to the subject (a) about 8 μg/kg, 16 μg/kg, 24 μg/kg, or 32 μg/kg of the IL-2 conjugate, and (b) pembrolizumab, wherein the IL-2 conjugate comprises the amino acid sequence of SEQ ID NO: 1 wherein the amino acid at position P64 is replaced by the structure of Formula (IA): 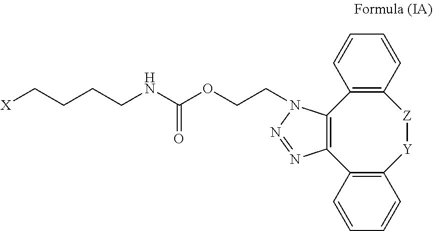
wherein:
Z is CH₂ and Y is 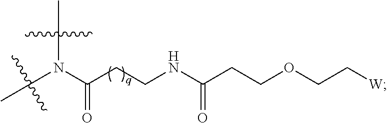
Y is CH₂ and Z is 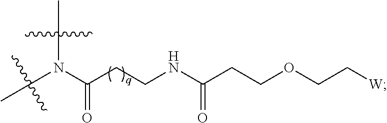
Z is CH₂ and Y is 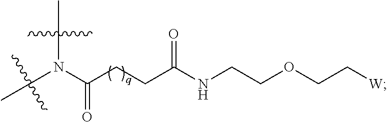
or
Y is CH₂ and Z is 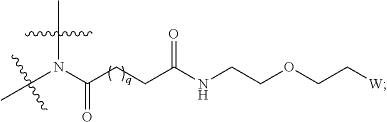
W is a PEG group having an average molecular weight of about 25 kDa-35 kDa;
q is 1, 2, or 3;
X is an L-amino acid having the structure: 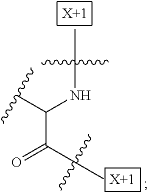
X−1 indicates the point of attachment to the preceding amino acid residue; and
X+1 indicates the point of attachment to the following amino acid residue.

3. Use of an IL-2 conjugate for the manufacture of a medicament for a method of treating a cancer in a subject in need thereof, the method comprising administering to the subject (a) about 8 μg/kg, 16 μg/kg, 24 μg/kg, or 32 μg/kg of the IL-2 conjugate, and (b) pembrolizumab, wherein the IL-2 conjugate comprises the amino acid sequence of SEQ ID NO: 1 wherein the amino acid at position P64 is replaced by the structure of Formula (IA): 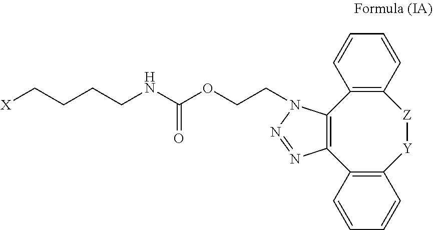
wherein:
Z is CH₂ and Y is 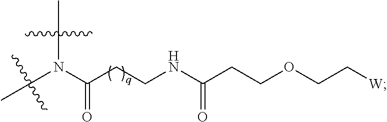
Y is CH₂ and Z is 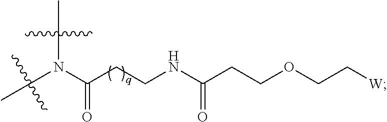
Z is CH₂ and Y is 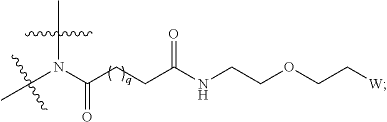
or
Y is CH₂ and Z is 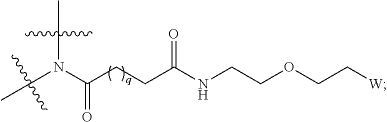
W is a PEG group having an average molecular weight of about 25 kDa-35 kDa;
q is 1, 2, or 3;
X is an L-amino acid having the structure: 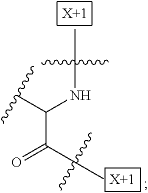
X−1 indicates the point of attachment to the preceding amino acid residue; and
X+1 indicates the point of attachment to the following amino acid residue.

4. The method, IL-2 conjugate for use, or use of any one of claims 1-3, comprising administering to the subject about 8 μg/kg of the IL-2 conjugate.

5. The method, IL-2 conjugate for use, or use of any one of claims 1-3, comprising administering to the subject about 16 μg/kg of the IL-2 conjugate.

6. The method, IL-2 conjugate for use, or use of any one of claims 1-3, comprising administering to the subject about 24 μg/kg of the IL-2 conjugate.

7. The method, IL-2 conjugate for use, or use of any one of claims 1-3, comprising administering to the subject about 32 μg/kg of the IL-2 conjugate.

8. The method, IL-2 conjugate for use, or use of any one of claims 1-7, wherein in the IL-2 conjugate Z is CH₂ and Y is

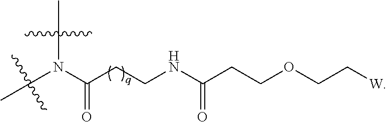

9. The method, IL-2 conjugate for use, or use of any one of claims 1-7, wherein in the IL-2 conjugate Y is CH₂ and Z is

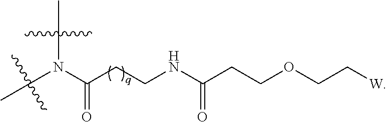

10. The method, IL-2 conjugate for use, or use of any one of claims 1-7, wherein in the IL-2 conjugate Z is CH₂ and Y is

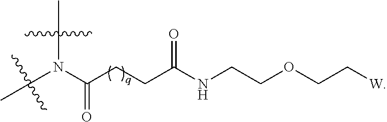

11. The method, IL-2 conjugate for use, or use of any one of claims 1-7, wherein in the IL-2 conjugate Y is CH₂ and Z is

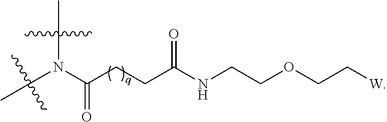

12. The method, IL-2 conjugate for use, or use of any one of claims 1-11, wherein in the IL-2 conjugate the PEG group has an average molecular weight of about 30 kDa.

13. The method, IL-2 conjugate for use, or use of any one of claims 1-7, wherein the structure of Formula (IA) has the structure of Formula (IVA) or Formula (VA), or is a mixture of Formula (IVA) and Formula (VA): 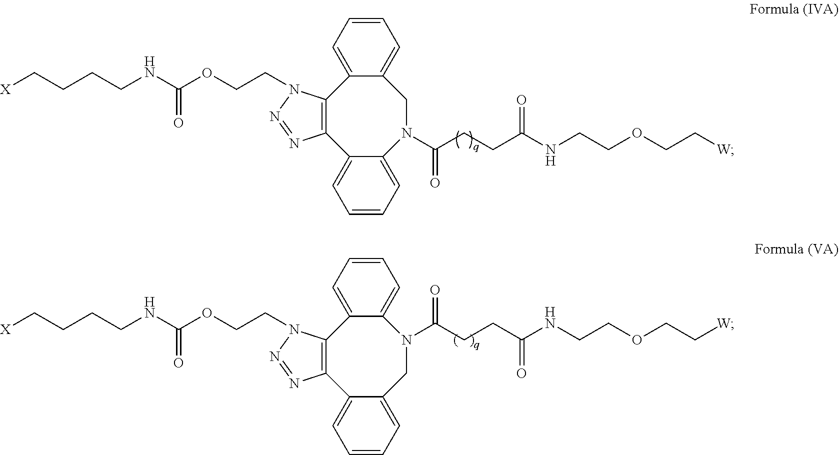
wherein:
W is a PEG group having an average molecular weight of about 25 kDa-35 kDa; and
q is 1, 2, or 3.

14. The method, IL-2 conjugate for use, or use of any one of claims 1-7, wherein the structure of Formula (IA) has the structure of Formula (XIIA) or Formula (XIIIA), or is a mixture of Formula (XIIA) and Formula (XIIIA) 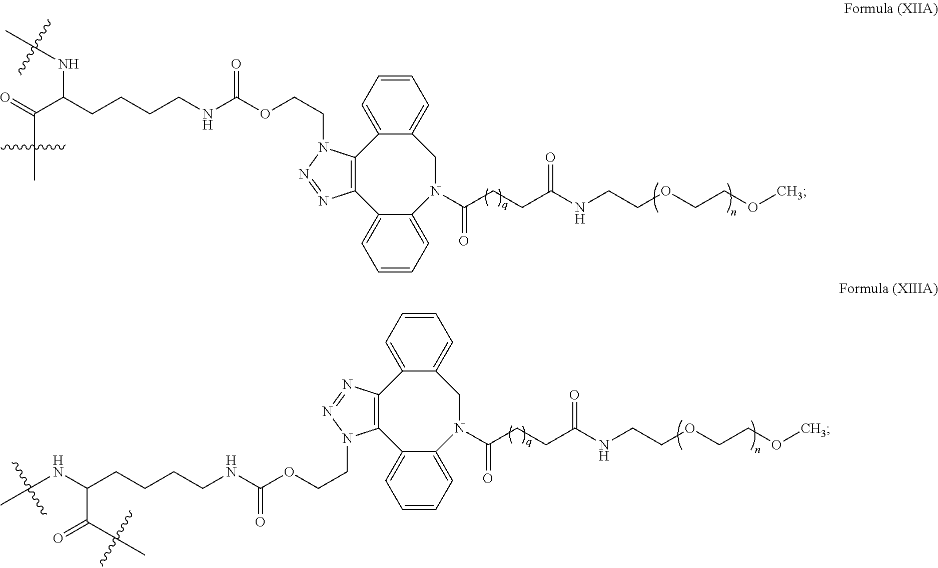
wherein:
n is an integer such that —(OCH₂CH₂)ₙ—OCH₃ has a molecular weight of about 30 kDa;
q is 1, 2, or 3; and
the wavy lines indicate covalent bonds to amino acid residues within SEQ ID NO: 1 that are not replaced.

15. The method, IL-2 conjugate for use, or use of any one of claims 1-14, wherein q is 1.

16. The method, IL-2 conjugate for use, or use of any one of claims 1-14, wherein q is 2.

17. The method, IL-2 conjugate for use, or use of any one of claims 1-14, wherein q is 3.

18. The method, IL-2 conjugate for use, or use of any one of claims 1-17, wherein the subject has a solid tumor cancer.

19. The method, IL-2 conjugate for use, or use of any one of claims 1-18, wherein the subject has a metastatic solid tumor.

20. The method, IL-2 conjugate for use, or use of any one of claims 1-19, wherein the subject has an advanced solid tumor.

21. The method, IL-2 conjugate for use, or use of any one of claims 1-17, wherein the subject has a liquid tumor.

22. The method, IL-2 conjugate for use, or use of any one of claims 1-21, wherein the subject has refractory cancer.

23. The method, IL-2 conjugate for use, or use of any one of claims 1-22, wherein the subject has relapsed cancer.

24. The method, IL-2 conjugate for use, or use of any one of claims 1-23, wherein the cancer is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, gastric cancer, colon cancer, colorectal cancer (CRC), cervical cancer, hepatocellular carcinoma (HCC), Merkel cell carcinoma (MCC), melanoma, small cell lung cancer (SCLC), esophageal, esophageal squamous cell carcinoma (ESCC), glioblastoma, mesothelioma, breast cancer, triple-negative breast cancer, prostate cancer, castrate-resistant prostate cancer, metastatic castrate-resistant prostate cancer, or metastatic castrate-resistant prostate cancer having DNA damage response (DDR) defects, bladder cancer, ovarian cancer, tumors of moderate to low mutational burden, cutaneous squamous cell carcinoma (CSCC), squamous cell skin cancer (SCSC), tumors of low- to non-expressing PD-L1, tumors disseminated systemically to the liver and CNS beyond their primary anatomic originating site, and diffuse large B-cell lymphoma (DLBCL).

25. The method, IL-2 conjugate for use, or use of any one of claims 1-24, wherein CD8+ cells are expanded at least 1.5-fold.

26. The method, IL-2 conjugate for use, or use of any one of claims 1-25, wherein NK cells are expanded at least about 5-fold.

27. The method, IL-2 conjugate for use, or use of any one of claims 1-26, wherein eosinophils are expanded no more than about 2-fold.

28. The method, IL-2 conjugate for use, or use of any one of claims 1-27, wherein CD4+ cells are expanded no more than about 2-fold.

29. The method, IL-2 conjugate for use, or use of any one of claims 1-28, wherein the expansion of CD8+ cells and/or NK cells is greater than the expansion of CD4+ cells and/or eosinophils.

30. The method, IL-2 conjugate for use, or use of any one of claims 1-29, wherein the IL-2 conjugate does not cause dose-limiting toxicity.

31. The method, IL-2 conjugate for use, or use of any one of claims 1-30, wherein the IL-2 conjugate does not cause severe cytokine release syndrome.

32. The method, IL-2 conjugate for use, or use of any one of claims 1-31, wherein the IL-2 conjugate does not cause vascular leak syndrome.

33. The method, IL-2 conjugate for use, or use of any one of claims 1-32, wherein the IL-2 conjugate is administered to the subject about once every two weeks, about once every three weeks, or about once every 4 weeks.

34. The method, IL-2 conjugate for use, or use of any one of claims 1-33, wherein the IL-2 conjugate and pembrolizumab are administered to the subject about once every two weeks, about once every three weeks, or about once every 4 weeks.

35. The method, IL-2 conjugate for use, or use of any one of claims 1-34, wherein the IL-2 conjugate is a pharmaceutically acceptable salt, solvate, or hydrate.

36. The method, IL-2 conjugate for use, or use of any one of claims 1-35, wherein pembrolizumab is administered at a dose of about 200 mg every 3 weeks.

37. The method, IL-2 conjugate for use, or use of any one of claims 1-36, wherein the IL-2 conjugate and pembrolizumab are administered separately.

38. The method, IL-2 conjugate for use, or use of claim 37, wherein the IL-2 conjugate and pembrolizumab are administered sequentially.

39. The method, IL-2 conjugate for use, or use of claim 37 or 38, wherein the IL-2 conjugate is administered before pembrolizumab.

40. The method, IL-2 conjugate for use, or use of claim 37 or 38, wherein the IL-2 conjugate is administered after pembrolizumab.

41. The method, IL-2 conjugate for use, or use of any one of claims 1-40, wherein the IL-2 conjugate is administered to the subject by subcutaneous administration.

42. The method, IL-2 conjugate for use, or use of any one of claims 1-40, wherein the IL-2 conjugate is administered to the subject by intravenous administration.

43. The method, IL-2 conjugate for use, or use of any one of claims 1-40 and 42, wherein the IL-2 conjugate and pembrolizumab are administered to the subject by intravenous administration.

44. The method, IL-2 conjugate for use, or use of any one of claims 1-43, wherein the subject has basal cell carcinoma.

45. The method, IL-2 conjugate for use, or use of any one of claims 1-43, wherein the subject has squamous cell carcinoma, optionally wherein the squamous cell carcinoma is head and neck squamous cell carcinoma.

46. The method, IL-2 conjugate for use, or use of any one of claims 1-43, wherein the subject has colorectal cancer.

47. The method, IL-2 conjugate for use, or use of any one of claims 1-43, wherein the subject has melanoma.
